(19) United States
(12) Patent Application Publication
(10) Pub. No.: US 2025/0073319 A1
(43) Pub. Date: Mar. 6, 2025

(54) LYOPHILIZED ENPP1 POLYPEPTIDE FORMULATIONS AND USES THEREOF

(71) Applicant: Inozyme Pharma, Inc., Boston, MA (US)

(72) Inventor: Steven Jungles, Key West, FL (US)

(21) Appl. No.: 18/612,571

(22) Filed: Mar. 21, 2024

Related U.S. Application Data
(63) Continuation of application No. PCT/US22/76969, filed on Sep. 23, 2022.
(60) Provisional application No. 63/248,303, filed on Sep. 24, 2021.

(51) Int. Cl. A61K 38/46; C12N 9/14

(52) U.S. Cl. CPC A61K 38/46; C12N 9/14; C12Y 306/01009

(57) ABSTRACT

In certain aspects, the present invention provides novel lyophilized formulations of ENPP1 polypeptides, as well as methods for using such lyophilized formulations and reconstituted formulations of ENPP1 to treat an indication associated an ENPP1 deficiency. The formulations and methods provided herein are useful in treating diseases associated with an ENPP1 deficiency such as pathological calcification or pathological ossification.

### CROSS REFERENCE

This U.S. patent application is a continuation application of International Patent Application No. PCT/US2022/076969, filed on Sep. 23, 2022, which claims priority to U.S. provisional patent application No. 63/248,303 filed on Sep. 24, 2021, the content of which is herein incorporated by reference in its entirety.

### SEQUENCE LISTING

This application contains a Sequence Listing which has been submitted electronically as a WIPO Standard ST.26 XML file via Patent Center, created on Mar. 21, 2024, is entitled “4427-11901.xml” and is 137 KB bytes in size. The sequence listing is incorporated herein by reference in its entirety.

### BACKGROUND OF THE INVENTION

The human ectonucleotide pyrophosphatase (ENPP) protein family comprises seven extracellular, glycosylated proteins (i.e., ENPP1-ENPP7) that hydrolyze phosphodiester bonds. ENPPs are cell-surface enzymes, with the exception of ENPP2, which is exported to the plasma membrane but is cleaved by furin and released into the extracellular fluid. The ENPP enzymes have high degrees of sequence and structural homology but exhibit a diverse substrate specificity encompassing nucleotides to lipids.

ENPP1 (also known as PC-1) and ENPP3 are type 2 extracellular membrane-bound glycoprotein located on the mineral-depositing matrix vesicles of osteoblasts and chondrocytes, and hydrolyze extracellular nucleotides (principally ATP) into adenosine monophosphate (AMP) and inorganic pyrophosphate (PPi). PPi functions as a potent inhibitor of ectopic tissue mineralization by binding to nascent hydroxyapatite (HA) crystals, thereby preventing the further growth of these crystals. ENPP1 generates PPi via hydrolysis of nucleotide triphosphates (NTPs).

Ectopic tissue mineralization is associated with numerous human diseases, including chronic joint disease and acutely fatal neonatal syndromes. To prevent unwanted tissue calcification, factors that promote and inhibit tissue mineralization must be kept in tight balance. The balance of extracellular inorganic pyrophosphate (PPi) and phosphate (Pi) is an important regulator of ectopic tissue mineralization.

### SUMMARY OF THE INVENTION

This disclosure features, among other things, lyophilized formulations comprising an ENPP1 polypeptide. Such formulations are characterized by, among other things, marked stability across a wide range of temperatures. Accordingly, in one aspect, the disclosure features a lyophilized polypeptide formulation, wherein the formulation comprises an ENPP1 polypeptide.

In some embodiments, a formulation described herein comprises a buffering agent. In some embodiments, the buffering agent maintains a pH range from pH 6-7 when reconstituted in solution. In some embodiments, the buffering agent maintains a pH range from pH 7-8 when reconstituted in solution. In some embodiments, the buffering agent maintains a pH range from pH 6-8 when reconstituted in solution. In some embodiments, the buffering agent is selected from the group consisting of succinate, citrate, bicarbonate, phosphate, tris, or glycylglycine. In some embodiments, the buffering agent is succinate, citrate, or phosphate. In some embodiments, the buffering agent is citrate. In some embodiments, the buffering agent is succinate. In some embodiments, the buffering agent is phosphate. In some embodiments, the buffering agent comprises a concentration ranging from 5 mM to 100 mM when reconstituted in solution. In some embodiments, the buffering agent increases the onset temperature of aggregate formation. In some embodiments, the buffering agent increases the onset temperature of aggregate formation by at least 2° C. In some embodiments, the buffering agent decreases formation of high molecular weight species. In some embodiments, the buffering agent decreases formation of high molecular weight species by at least 10%, 15%, 20%, 25%, 30%, 35%, 40%, 45%, or 50%.

In some embodiments, a formulation described herein comprises one or more pharmaceutically acceptable additives. In some embodiments, such a pharmaceutically acceptable additive is or comprises stabilizing agents, amino acids, salts, metal ions, and surfactants. In some embodiments, the stabilizing agent is or comprises a sugar, a carbohydrate, or a polysaccharide. In some embodiments, such a sugar is selected from the group consisting of sucrose, trehalose, mannose, maltose, lactose, glucose, raffinose, cellobiose, gentiobiose, isomaltose, arabinose, glucosamine, fructose, mannitol, or sorbitol. In some embodiments, the one or more pharmaceutically acceptable additives is or comprises mannitol. In some embodiments, the pharmaceutically acceptable additive is or comprises sucrose. In some embodiments, the pharmaceutically acceptable additive is or comprises an amino acid. In some embodiments, such an amino acid is selected from the group consisting of glycine, arginine, histidine, alanine, proline, serine, and glutamic acid. In some embodiments, the amino acid is arginine. In some embodiments, the pharmaceutically acceptable additive is a salt, such as, but not limited to, sodium chloride (NaCl), Calcium chloride (CaCl₂)). Zinc chloride (ZnCl₂), and/or Magnesium chloride (MgCl₂). In some embodiments, the salt is calcium chloride (CaCl₂)). In some embodiments, said pharmaceutically acceptable additive is a surfactant, such as, but not limited to, a polysorbate, poloxamer, triton, sodium dodecyl sulfate, sodium laurel sulfate, sodium octyl glycoside, laurylsulfobetaine, myristyl-sulfobetaine, linoleyl-sulfobetaine, stearyl-sulfobetaine, laurylsarcosine, myristyl-sarcosine, linoleyl-sarcosine, stearyl-sarcosine, linoleyl-betaine, myristyl-betaine, cetyl-betaine, lauroam idopropyl-betaine, cocam idopropyl-betaine, linoleamidopropyl-betaine, myristam idopropyl-betaine, palm idopropyl-betaine, isostearam idopropyl-betaine, myristam idopropyl-dimethylamine, palm idopropyldimethylamine, isostearam idopropyl-dimethylamine, sodium methyl cocoyl-taurate, disodium methyl oleyl-taurate, dihydroxypropyl PEG 5 linoleammonium chloride, polyethylene glycol, polypropylene glycol, polysorbate 20, polysorbate 21, polysorbate 40, polysorbate 60, polysorbate 61, polysorbate 65, polysorbate 80, polysorbate 81, polysorbate 85, polysorbate 188, PEG3350, and mixtures thereof. In some embodiments, the surfactant is selected from the group consisting of polysorbate 20 (PS20), polysorbate 80 (PS80), or polysorbate 188 (PS188). In some embodiments, the surfactant is polysorbate 20 (PS20).

In some embodiments, a formulation described herein comprises a buffering agent, a stabilizing agent, a salt, an amino acid, and a surfactant. In some embodiments, the buffering agent is a citrate or a succinate, the stabilizing agent is sucrose or mannitol, the salt is calcium chloride (CaCl₂)), and the surfactant is Polysorbate 20 (PS20).

In some embodiments, the pH of a formulation described herein (e.g., in the case of lyophilized formulations, when reconstituted in a reconstitution solution or sterile water) is pH 6.2 to pH 6.5. In some embodiments, the pH of a formulation described herein (e.g., in the case of lyophilized formulations, when reconstituted in a reconstitution solution or sterile water) is between about pH 6.0 to about pH 7.0, inclusive of pH 6.0 and pH 7.0.

In some embodiments, a formulation described herein comprises about 15 to about 25 mM of citrate.

In some embodiments, a formulation described herein comprises about 75 mM to about 95 mM each of sucrose, mannitol, or combinations thereof.

In some embodiments, a formulation described herein comprises about 100 mM to about 300 mM of sucrose.

In some embodiments, a formulation described herein comprises about 1 mM to about 3 mM of calcium chloride.

In some embodiments, the formulation comprises a surfactant. The surfactant can be or comprise, e.g., Polysorbate 20 (PS20), Polysorbate 80 (PS80), or poloxamer Px188. In some embodiments, the surfactant is or comprises PS20. In some embodiments, the surfactant is present at a concentration ranging from about 0.02% to about 0.10% (w/v) when reconstituted in solution. In some embodiments, a formulation described herein comprises about 0.005% to about 0.1% (w/v) of a surfactant. In some embodiments, a formulation described herein comprises about 0.005% to about 0.1% (w/v) of PS20. In some embodiments, the surfactant increases resistance to physical stress.

In some embodiments, the pharmaceutically acceptable additive or additives have a concentration ranging from about 5 mM to about 300 mM, when reconstituted in solution (e.g., with a reconstitution buffer or sterile water).

In some embodiments, the one or more pharmaceutically acceptable additives decrease formation of high molecular weight species.

In some embodiments, the one or more pharmaceutically acceptable additives decrease formation of high molecular weight species by at least 10%, 15%, 20%, 25%, 30%, 35%, 40%, 45%, 50%.

In some embodiments, the formulation comprises an ENPP1 polypeptide cofactor, such as, but not limited to, calcium, zinc, and/or adenosine monophosphate. In some embodiments, the ENPP1 polypeptide cofactor is CaCl₂), CaSO₄, ZnCl₂, ZnSO₄, and/or adenosine monophosphate. In some embodiments, the ENPP1 polypeptide cofactor is CaCl₂) and/or adenosine monophosphate. In some embodiments, the ENPP1 polypeptide cofactor is present at a concentration ranging from about 1 mM to about 10 mM when reconstituted in solution. In some embodiments, the ENPP1 polypeptide cofactor decreases formation of high molecular weight species by at least 10%, 15%, 20%, 25%, 30%, 35%, 40%, 45%, 50%.

In some embodiments, the formulations described herein comprise or are characterized by homogenous particulate size. In some embodiments, the formulations described herein comprise a twofold, threefold, fourfold, fivefold, sixfold, sevenfold, eightfold, ninefold, or tenfold reduction in larger sized particulates as determined by micro flow imaging analysis.

In some embodiments of any of the formulations described herein, the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO: 13, SEQ ID NO: 14, or SEQ ID NO:15.

In some embodiments, the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO:2.

In some embodiments, the ENPP1 polypeptide is a fusion protein comprising a soluble ENPP1 polypeptide domain and one or more heterologous protein portions. In some embodiments, the heterologous protein portion increases the circulating half-life of the soluble ENPP1 polypeptide in a mammal. In some embodiments, the heterologous protein portion comprises an Fc domain. In some embodiments, the Fc domain comprises an amino acid sequence that is at least 75%, 80%, 85%, 90%, 91%, 92%, 93%, 94%, 95%, 96%, 97%, 98%, 99%, or more identical to the amino acid sequence of SEQ ID NO:12. In some embodiments, the ENPP1 polypeptide is an ENPP1-Fc fusion protein. In some embodiments, the ENPP1 polypeptide further comprises a heterologous moiety. In some embodiments, the heterologous moiety is selected from the group consisting of a glycosylated amino acid, a PEGylated amino acid, a farnesylated amino acid, an acetylated amino acid, a biotinylated amino acid, and a lipid moiety.

In some embodiments, the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO: 13. In some embodiments, the ENPP1 polypeptide is at least 90% identical to SEQ ID NO:13. In some embodiments, the ENPP1 polypeptide is at least 95% identical to SEQ ID NO:13. In some embodiments, the ENPP1 polypeptide is at least 99% identical to SEQ ID NO:13. In some embodiments, the ENPP1 polypeptide comprises SEQ ID NO:13. In some embodiments, the ENPP1 polypeptide consists of SEQ ID NO:13. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO: 9. In some embodiments, the ENPP1 polypeptide consists of or comprises SEQ ID NO: 10. In some embodiments, the ENPP1 polypeptide consists of or comprises SEQ ID NO:11.

In some embodiments, the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO:14. In some embodiments, the ENPP1 polypeptide is at least 90% identical to SEQ ID NO:14. In some embodiments, the ENPP1 polypeptide is at least 95% identical to SEQ ID NO:14. In some embodiments, the ENPP1 polypeptide is at least 99% identical to SEQ ID NO:14. In some embodiments, the ENPP1 polypeptide comprises SEQ ID NO:14. In some embodiments, the ENPP1 polypeptide consists of SEQ ID NO:14. In some embodiments, the ENPP1 polypeptide comprises an Fc domain. The Fc domain can comprise or consist of the amino acid sequence depicted in SEQ ID NO:12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO: 9. In some embodiments, the ENPP1 polypeptide consists of or comprises SEQ ID NO: 10. In some embodiments, the ENPP1 polypeptide consists of or comprises SEQ ID NO:11.

In some embodiments, the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO:15. In some embodiments, the ENPP1 polypeptide is at least 90% identical to SEQ ID NO:15. In some embodiments, the ENPP1 polypeptide is at least 95% identical to SEQ ID NO:15. In some embodiments, the ENPP1 polypeptide is at least 99% identical to SEQ ID NO:15. In some embodiments, the ENPP1 polypeptide comprises SEQ ID NO:15. In some embodiments, the ENPP1 polypeptide consists of SEQ ID NO:15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO: 9. In some embodiments, the ENPP1 polypeptide consists of or comprises SEQ ID NO: 10. In some embodiments, the ENPP1 polypeptide consists of or comprises SEQ ID NO:11.

In some embodiments, the formulation comprises an ENPP1 polypeptide, a buffering agent, a pharmaceutically acceptable salt, a stabilizing agent, a surfactant, and one or more pharmaceutically acceptable additives. In some embodiments, the buffering agent is a citrate buffering agent. The citrate buffering agent can be, e.g., trisodium citrate dihydrate. In some embodiments, the pharmaceutically acceptable salt is calcium chloride. In some embodiments, the stabilizing agent is a sugar, such as sucrose. In some embodiments, the one or more pharmaceutically acceptable additives is or comprises mannitol. In some embodiments, the ENPP1 polypeptide comprises or consists of SEQ ID NO:13. In some embodiments, the ENPP1 polypeptide comprises or consists of SEQ ID NO:14. In some embodiments, the ENPP1 polypeptide comprises or consists of SEQ ID NO: 15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO: 11. In some embodiments, the ENPP1 polypeptide consists of or comprises SEQ ID NO:9. In some embodiments, the ENPP1 polypeptide consists of or comprises SEQ ID NO:10. In some embodiments, the ENPP1 polypeptide consists of or comprises SEQ ID NO:11.

In some embodiments, the formulation comprises: an ENPP1 polypeptide, a citrate buffering agent, calcium chloride, sucrose, and polysorbate. In some embodiments, the ENPP1 polypeptide comprises or consists of SEQ ID NO: 13. In some embodiments, the ENPP1 polypeptide comprises or consists of SEQ ID NO: 14. In some embodiments, the ENPP1 polypeptide comprises or consists of SEQ ID NO: 15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO:9. In some embodiments, the ENPP1 polypeptide consists of or comprises SEQ ID NO: 10. In some embodiments, the ENPP1 polypeptide consists of or comprises SEQ ID NO: 11.

In some embodiments, the formulation comprises: an ENPP1 polypeptide, trisodium citrate dihydrate, calcium chloride dihydrate, sucrose, and polysorbate 20. In some embodiments, the ENPP1 polypeptide comprises or consists of SEQ ID NO: 13. In some embodiments, the ENPP1 polypeptide comprises or consists of SEQ ID NO:14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO:12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO:9. In some embodiments, the ENPP1 polypeptide consists of or comprises SEQ ID NO:10. In some embodiments, the ENPP1 polypeptide consists of or comprises SEQ ID NO:11.

In some embodiments, the formulation comprises: an ENPP1 polypeptide, a citrate buffering agent, calcium chloride, sucrose, mannitol, and polysorbate. In some embodiments, the ENPP1 polypeptide comprises or consists of SEQ ID NO:13. In some embodiments, the ENPP1 polypeptide comprises or consists of SEQ ID NO: 14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO:12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO: 11. In some embodiments, the ENPP1 polypeptide consists of or comprises SEQ ID NO:9. In some embodiments, the ENPP1 polypeptide consists of or comprises SEQ ID NO:10. In some embodiments, the ENPP1 polypeptide consists of or comprises SEQ ID NO:11.

In some embodiments, the formulation comprises: an ENPP1 polypeptide, trisodium citrate dihydrate, calcium chloride dihydrate, sucrose, D (−) mannitol, and polysorbate 20. In some embodiments, the ENPP1 polypeptide comprises or consists of SEQ ID NO:13. In some embodiments, the ENPP1 polypeptide comprises or consists of SEQ ID NO:14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO: 11. In some embodiments, the ENPP1 polypeptide consists of or comprises SEQ ID NO:9. In some embodiments, the ENPP1 polypeptide consists of or comprises SEQ ID NO:10. In some embodiments, the ENPP1 polypeptide consists of or comprises SEQ ID NO:11.

In some embodiments, the formulation comprises per 0.5 ml final reconstituted volume: about 25 mg of ENPP1 polypeptide, about 2.94 mg trisodium citrate dihydrate, about 0.15 mg calcium chloride dihydrate, about 30 mg of sucrose, about 7.5 mg D (−) mannitol, and about 0.25 mg polysorbate (e.g., PS20). In some embodiments, the ENPP1 polypeptide comprises or consists of SEQ ID NO:13. In some embodiments, the ENPP1 polypeptide comprises or consists of SEQ ID NO: 14. In some embodiments, the ENPP1 polypeptide comprises or consists of SEQ ID NO: 15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO:10, or SEQ ID NO:11. In some embodiments, the ENPP1 polypeptide consists of or comprises SEQ ID NO:9. In some embodiments, the ENPP1 polypeptide consists of or comprises SEQ ID NO: 10. In some embodiments, the ENPP1 polypeptide consists of or comprises SEQ ID NO:11.

In some embodiments, the formulation can be reconstituted in a sterile injectable solution. In some embodiments, the formulation can be reconstituted in a reconstitution solution or sterile water. In some embodiments, the reconstitution solution comprises a pharmaceutically acceptable carrier and/or an additive. In some embodiments, the pharmaceutically acceptable carrier is selected from saline solution, purified water, or sterile water for injection. In some embodiments, the formulation is completely reconstituted within a period of less than 100 seconds, 80 seconds, 70 seconds, 68 seconds, 65 seconds, or 60 seconds.

In some embodiments, the reconstituted formulation comprises: at least or about 50 mg/mL of the ENPP1 polypeptide, about 20 mM citrate at about pH 6.3, about 2 mM calcium chloride, about 175 mM sucrose, about 82 mM mannitol, and about 0.05% w/v polysorbate. In some embodiments, the ENPP1 polypeptide comprises or consists of SEQ ID NO:13. In some embodiments, the ENPP1 polypeptide comprises or consists of SEQ ID NO:14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO:11.

In some embodiments, the reconstituted formulation comprises: at least or about 50 mg/mL of the ENPP1 polypeptide, about 20 mM citrate at about pH 6.3, about 2 mM calcium chloride, about 175 mM sucrose, about 82 mM (D) mannitol, and about 0.05% w/v polysorbate 20. In some embodiments, the ENPP1 polypeptide comprises or consists of SEQ ID NO:13. In some embodiments, the ENPP1 polypeptide comprises or consists of SEQ ID NO:14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO: 12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO:11.

In some embodiments, the reconstituted formulation comprises: at least or about 50 mg/mL of the ENPP1 polypeptide, about 20 mM citrate at about pH 6.3, about 88 mM sucrose, about 82 mM mannitol, about 2 mM calcium chloride, and about 0.05% polysorbate 20. In some embodiments, the ENPP1 polypeptide comprises or consists of SEQ ID NO:13. In some embodiments, the ENPP1 polypeptide comprises or consists of SEQ ID NO:14. In some embodiments, the ENPP1 polypeptide comprises or consists of SEQ ID NO:15. In some embodiments, the ENPP1 polypeptide comprises an Fc domain. The Fc domain can comprise or consist of the amino acid sequence depicted in SEQ ID NO:12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO:11.

In some embodiments, the reconstituted formulation comprises: at least or about 50 mg/mL of the ENPP1 polypeptide, about 20 mM citrate at about pH 6.3, about 263 mM sucrose, about 2 mM calcium chloride, and about 0.05% polysorbate 20. In some embodiments, the ENPP1 polypeptide comprises or consists of SEQ ID NO:13. In some embodiments, the ENPP1 polypeptide comprises or consists of SEQ ID NO: 14. In some embodiments, the ENPP1 polypeptide comprises or consists of SEQ ID NO: 15. In some embodiments, the ENPP1 polypeptide comprises an Fc domain. The Fc domain can comprise or consist of the amino acid sequence depicted in SEQ ID NO:12. In some embodiments, the ENPP1 polypeptide comprises a linker amino acid sequence joining the ENPP1 polypeptide portion and the Fc domain. In some embodiments, the linker amino acid sequence comprises or consists of the amino acid sequence LIN. In some embodiments, the ENPP1 polypeptide consists of or comprises SEQ ID NO:9, SEQ ID NO: 10, or SEQ ID NO:11.

In some embodiments, the lyophilized formulation exhibits long term stability at −80° C. to 40° C. In some embodiments, the lyophilized formulation has a shelf life of at least 3, 6, 12, 24, 36, 48, or 60 months. In some embodiments, the reconstituted formulation has a shelf life of at least 1, 2, 3, 4, 5, 6, 12, 18, 24, 48, or 60 hours.

In some embodiments, the lyophilized formulation is shelf stable for at least 36 months when stored at 5° C. In some embodiments, the formulation is shelf-stable for 48 months when stored at 5° C. The formulation is shelf-stable for at least 24 months when stored at room temperature (RT−25° C.). The formulation is shelf-stable for at least 36 months when stored at RT. The formulation is shelf-stable for 48 months when stored at RT. The formulation is shelf-stable for at least 6 months when stored at 40° C. The formulation is shelf-stable for 12 months when stored at 40° C.

In some embodiments, the reconstituted formulation is stable in concentrations ranging from 1 mg/ml to 50 mg/ml for at least 8 hours at RT. In some embodiments, the reconstituted formulation is stable in concentrations ranging from 1 mg/ml to 50 mg/ml for at least 24 hours at 5° C.

In some embodiments, the reconstituted formulation is stable in concentrations ranging from 1 mg/ml to 10 mg/ml for at least 12 hours at 5° C. In some embodiments, the reconstituted formulation is stable at 1 mg/ml for at least 12 hours at 5° C. In some embodiments, the reconstituted formulation is stable at 2.5 mg/ml for at least 12 hours at 5° C. In some embodiments, the reconstituted formulation is stable at 10 mg/ml for at least 12 hours at 5° C.

In some embodiments, the reconstituted formulation is stored in a vial. In some embodiments, the reconstituted formulation is stored in a syringe. In some embodiments, the reconstituted formulation when stored in a vial at 5° C. is stable for at least 12 hours. In some embodiments, the reconstituted formulation when stored in a syringe at 5° C. is stable for at least 8 hours.

Accordingly, the disclosure also features a vial comprising a lyophilized formulation comprising ENPP1, preferably ENPP1-Fc, and one or more of a buffer, a bulking agent, stabilizer, and/or a surfactant, wherein the vial comprises 1-100 mg, for example, 1 mg, 5 mg, 10 mg, 15 mg, 20 mg, 25 mg or 50 mg or 100 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 100 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 50 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 25 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 10 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 5 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 2.5 mg of ENPP1 or ENPP1-Fc.

In some embodiments, the vial comprises a lyophilized formulation comprising ENPP1, preferably ENPP1-Fc, and one or more of a buffer, a bulking agent, stabilizer, and/or a surfactant, wherein the vial comprises 1 mg of ENPP1 or ENPP1-Fc.

Accordingly, the disclosure also features a vial comprising a formulation comprising reconstituted ENPP1, preferably ENPP1-Fc, and one or more of a buffer, a bulking agent, stabilizer, and/or a surfactant, wherein the vial comprises 0.1 mg/ml-100 mg/ml, for example, 0.1 mg/ml, 1 mg/ml, 5 mg/ml, 10 mg/ml, 15 mg/ml, 20 mg/ml, 25 mg/ml or 50 mg/ml or 100 mg/ml of ENPP1 or ENPP1-Fc.

In some embodiments, the vial comprising a formulation comprising reconstituted ENPP1, preferably ENPP1-Fc, and one or more of a buffer, a bulking agent, stabilizer, and/or a surfactant, wherein the vial comprises 100 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 50 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 25 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 10 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 5 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 2.5 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 1 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 0.5 mg/ml of ENPP1 or ENPP1-Fc.

In some embodiments, the vial comprises a formulation comprising reconstituted ENPP1, preferably ENPP1-Fc, and one or more of a buffer, a bulking agent, stabilizer, and/or a surfactant, wherein the vial comprises 0.1 mg/ml of ENPP1 or ENPP1-Fc.

Accordingly, the disclosure also features a syringe comprising a lyophilized formulation comprising ENPP1, preferably ENPP1-Fc, and one or more of a buffer, a bulking agent, stabilizer, and/or a surfactant, wherein the vial comprises 0.1-100 mg, for example, 0.1 mg, 1 mg, 5 mg, 10 mg, 15 mg, 20 mg, 25 mg or 50 mg or 100 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 100 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 50 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 25 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 10 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 5 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 2.5 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 1 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 0.5 mg of ENPP1 or ENPP1-Fc.

In some embodiments, the syringe comprises a lyophilized formulation comprising ENPP1, preferably ENPP1-Fc, and one or more of a buffer, a bulking agent, stabilizer, and/or a surfactant, wherein the vial comprises 0.1 mg of ENPP1 or ENPP1-Fc.

Accordingly, the disclosure also features a syringe comprising a formulation comprising reconstituted ENPP1, preferably ENPP1-Fc, and one or more of a buffer, a bulking agent, stabilizer, and/or a surfactant, wherein the vial comprises 0.1 mg/ml-100 mg/ml, for example, 0.1 mg/ml, 1 mg/ml, 5 mg/ml, 10 mg/ml, 15 mg/ml, 20 mg/ml, 25 mg/ml or 50 mg/ml or 100 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 100 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 50 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 25 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 10 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 5 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 2.5 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 1 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 0.5 mg/ml of ENPP1 or ENPP1-Fc.

In some embodiments, the syringe comprises a formulation comprising reconstituted ENPP1, preferably ENPP1-Fc, and one or more of a buffer, a bulking agent, stabilizer, and/or a surfactant, wherein the vial comprises 0.1 mg/ml of ENPP1 or ENPP1-Fc.

In some embodiments, the lyophilized ENPP1, preferably ENPP1-Fc polypeptide is stored in a vial. In some embodiments, the vial comprises 0.1 mg of lyophilized ENPP1, preferably ENPP1-Fc polypeptide. In some embodiments, the vial comprises 0.5 mg of lyophilized ENPP1, preferably ENPP1-Fc polypeptide. In some embodiments, the vial comprises 1 mg of lyophilized ENPP1, preferably ENPP1-Fc polypeptide. In some embodiments, the vial comprises 5 mg of lyophilized ENPP1, preferably ENPP1-Fc polypeptide. In some embodiments, the vial comprises 10 mg of lyophilized ENPP1, preferably ENPP1-Fc polypeptide. In some embodiments, the vial comprises 15 mg of lyophilized ENPP1, preferably ENPP1-Fc polypeptide. In some embodiments, the vial comprises 25 mg of lyophilized ENPP1, preferably ENPP1-Fc polypeptide. In other embodiments, the vial comprises 50 mg of lyophilized ENPP1, preferably ENPP1-Fc polypeptide. In other embodiments, the vial comprises 100 mg of lyophilized ENPP1, preferably ENPP1-Fc polypeptide.

In some embodiments, the lyophilized ENPP1-Fc formulation is reconstituted for administration and is stored in a syringe. In some embodiments, the syringe comprises 100 mg/ml of ENPP1-Fc for administration or subsequent dilution. In some embodiments, the syringe comprises 50 mg/ml of ENPP1-Fc for administration or subsequent dilution. In some embodiments, the syringe comprises 25 mg/ml of ENPP1-Fc for administration or subsequent dilution. In some embodiments, the syringe comprises 20 mg/ml of ENPP1-Fc for administration or subsequent dilution. In some embodiments, the syringe comprises 15 mg/ml of ENPP1-Fc for administration or subsequent dilution. In some embodiments, the syringe comprises 10 mg/ml of ENPP1-Fc for administration or subsequent dilution. In some embodiments, the syringe comprises 5 mg/ml of ENPP1-Fc for administration or subsequent dilution. In some embodiments, the syringe comprises 4 mg/ml of ENPP1-Fc for administration or subsequent dilution. In some embodiments, the syringe comprises 3 mg/ml of ENPP1-Fc for administration or subsequent dilution. In some embodiments, the syringe comprises 2.5 mg/ml of ENPP1-Fc for administration or subsequent dilution. In some embodiments, the syringe comprises 2 mg/ml of ENPP1-Fc for administration or subsequent dilution. In some embodiments, the syringe comprises 1 mg/ml of ENPP1-Fc for administration or subsequent dilution. In some embodiments, the syringe comprises 0.5 mg/ml of ENPP1-Fc for administration or subsequent dilution. In some embodiments, the syringe comprises 0.1 mg/ml of ENPP1-Fc for administration or subsequent dilution.

In some embodiments, ENPP1-Fc formulation is administered to adults at a dosage ranging from 0.2 mg/kg to 1.8 mg/ml. In some embodiments, ENPP1-Fc formulation is administered to children and babies weighing over 1 kg at a dosage ranging from 0.2 mg/kg to 0.6 mg/kg. In some embodiments, ENPP1-Fc formulation is administered to premature babies weighing under 1 kg at a dosage ranging from 0.05 mg/kg to 0.2 mg/kg. In some embodiments, ENPP1-Fc formulation is administered to babies weighing over 1 kg at a dosage ranging of 0.2 mg/kg. In some embodiments, ENPP1-Fc formulation is administered to premature babies weighing under 1 kg at a dosage of 0.1 mg/kg

In some embodiments, any of the lyophilized formulations described herein comprises a therapeutically effective dose.

In some embodiments, any of the reconstituted formulations described herein comprises a therapeutically effective dose.

In yet another aspect, the disclosure features a method for generating or making any of the lyophilized formulations described herein. The method comprises admixing with an ENPP1 polypeptide (such as a fusion polypeptide comprising a catalytic domain of ENPP1 and an Fc region of an immunoglobulin) one or more of a buffering agent, a stabilizing agent, a salt, an amino acid, and a surfactant. In some embodiments, the method comprises admixing with the ENPP1 polypeptide a citrate buffer, sucrose, mannitol, calcium chloride, and polysorbate 20.

In yet another aspect, the disclosure features a method for generating a pharmaceutical solution comprising an ENPP1 polypeptide. The method comprises contacting any of the lyophilized polypeptide formulations described herein with a sterile reconstitution solution or sterile water to thereby generate a reconstituted solution comprising the ENPP1 polypeptide. In some embodiments, the reconstitution solution comprises a pharmaceutically acceptable carrier and/or an additive. In some embodiments, the pharmaceutically acceptable carrier is selected from saline solution, purified water, or sterile water for injection. In some embodiments, the formulation is completely reconstituted within a period of less than 100 seconds, 80 seconds, 70 seconds, 68 seconds, 65 seconds, or 60 seconds. In some embodiments, the reconstituted formulation comprises: at least or about 50 mg/mL of the ENPP1 polypeptide, about 20 mM citrate at about pH 6.3, about 2 mM calcium chloride, about 175 mM sucrose, about 82 mM mannitol, and about 0.05% w/v polysorbate. In some embodiments, the reconstituted formulation comprises: at least or about 50 mg/mL of the ENPP1 polypeptide, about 20 mM citrate at about pH 6.3, about 2 mM calcium chloride, about 175 mM sucrose, about 82 mM (D) mannitol, and about 0.05% w/v polysorbate 20. In some embodiments, the reconstituted formulation comprises: at least or about 50 mg/mL of the ENPP1 polypeptide, about 20 mM citrate at about pH 6.3, about 88 mM sucrose, about 82 mM mannitol, about 2 mM calcium chloride, and about 0.05% polysorbate 20. In some embodiments, the reconstituted formulation comprises: at least or about 50 mg/mL of the ENPP1 polypeptide, about 20 mM citrate at about pH 6.3, about 263 mM sucrose, about 2 mM calcium chloride, and about 0.05% polysorbate 20. In another aspect, the disclosure features a reconstituted solution made by any of the methods for generating a pharmaceutical solution described herein.

In yet another aspect, the disclosure features a method for preventing the progression of or reducing vascular calcification in a subject in need thereof. The method comprises: administering to the subject any of the reconstituted formulations described herein in an amount effective to prevent the progression of or reduce vascular calcification in the subject.

In another aspect, the disclosure features a method for preventing the progression of or reducing pathological calcification in a subject with ENPP1 Deficiency. In another aspect, the disclosure features a method for treating a subject with ENPP1 Deficiency. These methods comprise: administering to the subject any of the reconstituted formulations described herein in an amount effective to prevent the progression of or reduce pathological calcification in the subject.

In another aspect, the disclosure features a method for preventing the progression of or reducing pathological calcification in a subject with ABCC6 Deficiency. In another aspect, the disclosure features a method for treating a subject with ABCC6 Deficiency, such as PXE. These methods comprise: administering to the subject any of the reconstituted formulations described herein in an amount effective to prevent the progression of or reduce pathological calcification in the subject.

In some embodiments, the reconstituted formulation can be administered to the subject at a dose of about 0.1 mg per kilogram, about 0.2 mg per kilogram of the subject, about 0.6 mg per kilogram of the subject, or about 1.8 mg per kilogram of the subject.

In some embodiments, the reconstituted formulation can be administered to the subject at a dose of about 0.1 mg per kilogram, about 0.2 mg per kilogram, about 0.3 mg per kg, about 0.4 mg per kg, about 0.5 mg per kg, about 0.6 mg per kg, about 0.7 mg per kg, about 0.8 mg per kg, about 0.9 mg per kg, about 1 mg per kg, about 1.1 mg per kg, about 1.2 mg per kg, about 1.3 mg per kg, about 1.4 mg per kg, about 1.5 mg per kg, about 1.6 mg per kg, about 1.7 mg per kg or 1.8 mg/kg of the subject.

In yet another aspect, the disclosure features a method for preventing the progression of or reducing tissue calcification in a subject in need thereof, which method comprises administering to the subject any of the reconstituted formulations described herein in an amount effective to prevent the progression of or reduce tissue calcification in the subject. In some embodiments, the subject has or is suspected of having ENPP1 Deficiency. In some embodiments, the subject has or is suspected of having ABCC6 Deficiency (such as pseudoxanthoma elasticum (PXE)).

In yet another aspect, the disclosure features a method for preventing the progression of or reducing pathological ossification in a subject in need thereof. The method comprises administering to the subject any of the reconstituted formulations described herein in an amount effective to prevent the progression of or reduce tissue calcification in the subject. In some embodiments, the subject has or is suspected of having ENPP1 Deficiency. In some embodiments, the subject has or is suspected of having ABCC6 Deficiency (such as pseudoxanthoma elasticum (PXE)).

In yet another aspect, the disclosure features a method for increasing circulating pyrophosphate (PPi) in a subject in need thereof. The method comprises administering to the subject any of the reconstituted formulations described herein in an amount effective to increase circulating PPi in the subject. In some embodiments, the subject has or is suspected of having ENPP1 Deficiency. In some embodiments, the subject has or is suspected of having ABCC6 Deficiency (such as pseudoxanthoma elasticum (PXE)). The phrase “deficient for NPP1” or “ENPP1 deficiency” refers to a reduction in an amount of NPP1 protein or in NPP1 activity relative to a normal serum level of NPP1 protein or normal activity of NPP1, wherein such a reduction results in a disease or disorder of pathological calcification and/or pathological ossification and/or reduction in PPi levels. Such pathological diseases include but are not limited to GACI and ARHR2. ENPP1 deficiency, as used herein, does not refer to small reductions in an amount of NPP1 protein and/or NPP1 activity that do not result in a disease or disorder of pathological calcification and/or pathological ossification. The phrase “ABCC6 deficient patient” or “ABCC6 deficient subject” as used herein, refers to a patient having at least one pathogenic mutation in the ABCC6 gene that affects activity and/or expression of ABC66 protein.

In another aspect, the disclosure features a method for increasing pyrophosphatase activity in a subject in need thereof. The method comprises administering to the subject any of the reconstituted formulations described herein in an amount effective to increase circulating PPi in the subject. In some embodiments, the subject has or is suspected of having ENPP1 Deficiency. In some embodiments, the subject has or is suspected of having ABCC6 Deficiency (such as pseudoxanthoma elasticum (PXE)). The term “pyrophosphatase activity” refers to the ability of ENPP1 enzyme to hydrolyze a pyrophosphate bond between two phosphate groups in a molecule such as ATP.

In some embodiments of any of the methods described herein, the subject has a disorder involving pathological calcification. In some embodiments of any of the methods described herein, the subject has a disorder involving pathological ossification. In some embodiments of any of the methods described herein, the subject has or is at risk for developing pathological soft tissue calcification, arterial calcification, vascular calcification, chronic kidney disease (CKD), end stage renal disease (ESRD), calcific uremic arteriolopathy (CUA), calciphylaxis, ossification of the posterior longitudinal ligament (OPLL), or hypophosphatemic rickets.

In yet another aspect, the disclosure features a method for treating or ameliorating one or more symptoms of ENPP1 Deficiency in a subject, which method comprises administering to the subject any of the reconstituted formulations described herein, to ameliorate one or more symptoms of ENPP1 Deficiency in the subject.

In another aspect, the disclosure features a method for treating a subject with ENPP1 Deficiency. The method comprises administering to the subject any of the reconstituted formulations described herein, to thereby treat the subject.

In yet another aspect, the disclosure features a method for treating or ameliorating one or more symptoms of ABCC6 Deficiency in a subject, which method comprises administering to the subject any of the reconstituted formulations described herein in an amount effective to ameliorate one or more symptoms of ABCC6 Deficiency in the subject.

In another aspect, the disclosure features a method for treating a subject with ABCC6 Deficiency, which method comprises administering to the subject the reconstituted formulation in an amount effective to treat the subject.

In some embodiments of any of the methods described herein, the reconstituted formulation is administered parenterally. In some embodiments of any of the methods described herein, the reconstituted formulation is administered via subcutaneous injection. In some embodiments of any of the methods described herein, the reconstituted formulation is administered via intravenous injection. In some embodiments of any of the methods described herein, the reconstituted formulation is administered via intradermal injection. In some embodiments of any of the methods described herein, the reconstituted formulation is administered via intramuscular injection.

In some embodiments of any of the methods described herein, the reconstituted formulation is self-administered. In some embodiments of any of the methods described herein, the formulation is administered several times daily, every two days, three days, one week, or one month.

In some embodiments of any of the methods described herein, the second dose (or additional doses) of the formulation is/are administered after a suitable time interval of at least after two days, after four days, after a week, or after a month (from the first or prior dose).

### BRIEF DESCRIPTION OF THE DRAWINGS

FIG. 1 shows the full, unprocessed amino acid sequence of wild-type ENPP1 precursor protein (SEQ ID NO: 1). The cytosolic and transmembrane regions are underlined. Potential N-glycosylation sites are in bold. PSCAKE (residues 99-104; boxed) is the start of soluble ENPP1 protein portion which includes SMB1 (residues 104-144) and SMB2 (residues 145-189).

FIG. 2 illustrates Cytosolic domain (residues 1-77), Transmembrane domain (TM) (residues 77-100), somatomedin-B-like domain-1 (SMB1) (residues 100-142), somatomedin-B-like domain-2 (SMB2) (residues 142-187), catalytic domain and nuclease domain of human ENPP1.

FIG. 3 shows the amino acid sequence of a soluble ENPP1 polypeptide (SEQ ID NO: 2).

FIG. 4A and FIG. 4B show a multiple sequence alignment of various vertebrate soluble ENPP1 polypeptides and human soluble ENPP1 polypeptide (SEQ ID NOs: 4-8). The various soluble ENPP1 polypeptides correspond to the following species and represent regions of the specific NCBI accession number: Mouse (NCBI accession NP_001295256.1; SEQ ID NO: 4), Cow (NCBI accession NP_001193141; SEQ ID NO: 5), Rabbit (NCBI accession NP_001162404.1; SEQ ID NO:6), Human (NCBI accession NP_006199.2; SEQ ID NO: 1), and Baboon (NCBI accession NP_001076211.2; SEQ ID NO: 8).

FIG. 5 is a bar graph showing summary size exclusion chromatography (SEC) results for ENPP1-Fc formulations comprising various buffer components. Data are shown in terms of % high molecular weight species (HMW), or % main peak.

FIG. 6 is a bar graph showing percent recovery for reconstituted formulations of lyophilized ENPP1-Fc polypeptide formulations comprising various combinations of buffer and excipient components.

FIG. 7 is a bar graph showing summary enzymatic activity data for lyophilized ENPP1-Fc polypeptide formulations comprising various combinations of buffer and excipient components at 5° C. and 40° C. The activity data are presented in U/mg.

FIG. 8 is a bar graph showing summary of SEC data for lyophilized ENPP1-Fc polypeptide formulations comprising various combinations of buffer and excipient components at 5° C. and 40° C. Data are shown in terms of percent abundance of high molecular weight species (HMW), main peak species, or low molecular weight species (LMW).

FIG. 9A and FIG. 9B are bar graphs showing summary SEC data for lyophilized ENPP1-Fc polypeptide formulations comprising various buffer, excipient, and surfactant components. The depicted formulations were tested under stress conditions comprising agitation at 600 rpm (depicted as “agit”), or 5 rounds of freeze thaw cycling (i.e., storage at −80° C. for ≥60 minutes followed by thawing at room temperature; depicted as “F/T”). Data are shown in terms of % HMW (FIG. 9A) or % main peak (FIG. 9B).

FIG. 10 is a bar graph showing summary SEC data for lyophilized ENPP1-Fc polypeptide formulations comprising various cofactors in a succinate and sucrose buffer/additive background. The data represent percent abundance of HMW and main peak species at 5° C.

FIG. 11 is a bar graph showing summary SEC data for lyophilized ENPP1-Fc polypeptide formulations comprising various cofactors in a succinate, sucrose, and PS20 buffer/additive/surfactant background. The data represent percent abundance of HMW and main peak species incubated at 40° C. for ˜6 days.

FIGS. 12A and 12B are bar graphs showing summary SEC data for lyophilized ENPP1-Fc polypeptide formulations comprising various cofactors in a succinate, sucrose, and PS20 buffer/additive/surfactant background. The data represent percent abundance of HMW, LMW, and main peak species at 5° C. (FIG. 12B) or incubated at 40° C. for ˜6 days (FIG. 12A).

FIG. 13 is a bar graph showing summary osmolality data for lyophilized ENPP1-Fc polypeptide formulation samples.

FIG. 14 is a bar graph showing summary SEC data for lyophilized ENPP1-Fc polypeptide formulation samples comprising several preferred buffer/excipient/additive/surfactant combinations stored for up to 13 weeks at −75° C., 5° C., or 40° C. The data represent percent abundance of HMW and main peak species across the different formulations/conditions.

FIG. 15 is a plot showing summary SEC data for two preferred lyophilized ENPP1 polypeptide formulations (stability form A and stability form B) stored at either 5° C., 25° C., or 40° C. over a time period of three months.

FIG. 16 is a plot showing summary enzymatic activity data for two preferred lyophilized ENPP1 polypeptide formulations (stability form A and stability form B) stored at either 5° C., 25° C., or 40° C. over a time period of three months.

FIG. 17 is a plot showing summary SEC data for lyophilized ENPP1-Fc polypeptide formulations stored for different time periods at 5° C.

FIG. 18 is a plot showing summary SEC data for lyophilized ENPP1-Fc polypeptide formulations stored for different time periods at 25° C.

FIG. 19 is a plot showing summary SEC data for lyophilized ENPP1-Fc polypeptide formulations stored for different time periods at 40° C.

FIG. 20 is a plot showing summary enzymatic activity data for lyophilized ENPP1-Fc polypeptide formulations stored for different time periods such as 5° C., 25° C. and 40° C.

FIG. 21 is a bar graph showing percent recovery for reconstituted formulations of lyophilized ENPP1-Fc polypeptide stored in syringe or vial at different time intervals.

FIG. 22 is a bar graph showing summary enzymatic activity data for lyophilized ENPP1-Fc polypeptide stored in syringe or vial at different time intervals.

FIG. 23 is a plot showing percent recovery for reconstituted formulations of lyophilized ENPP1-Fc polypeptide stored in syringe or vial at different time intervals.

FIG. 24 is a plot showing summary enzymatic activity data for lyophilized ENPP1-Fc polypeptide stored in syringe or vial at different time intervals

### DETAILED DESCRIPTION

### 1. Overview

The application provides stable, lyophilized formulations comprising ENPP1 polypeptides and their uses in treating diseases associated with ENPP1. In certain aspects, the disclosure relates to lyophilized formulations of ENPP1 polypeptides and uses thereof (e.g., of treating, preventing, or reducing the progression rate and/or severity of pathologic calcification and/or ossification or one or more complications of pathologic calcification and/or ossification). The application provides lyophilized formulations that demonstrate enhanced stability, increased shelf-life and lower levels of high molecular weight species (HMW) and aggregates. The HMW species maybe dimers, tetramers, and high order aggregates (HMW1/HMW2).

ENPP1 polypeptides have been shown to be effective in treating certain diseases of ectopic tissue calcification. ENPP1-Fc has been shown to reduce generalized arterial calcifications in a mouse model for GACI (generalized arterial calcification of infants), which is a severe disease occurring in infants and involving extensive arterial calcification (Albright, et al., 2015, Nature Comm. 10006). Fusion proteins of ENPP1 have also been described to treat diseases of severe tissue calcification (see, e.g., PCT Application Publication Nos. WO 2014/126965 and WO 2016/187408), and a fusion protein of ENPP1 comprising a negatively-charged bone-targeting domain has been described to treat GACI (PCT Application Publication Nos. WO 2011/113027 and WO 2012/125182).

Mammal ENPP1 polypeptides, mutants, or mutant fragments thereof, have been previously disclosed in International PCT Application Publications No. WO/2014/126965—Braddock et al., WO/2016/187408—Braddock et al., WO/2017/087936—Braddock et al., and WO2018/027024—Braddock et al., all of which are incorporated by reference in their entireties herein.

### 2. Lyophilized Formulations

In certain aspects, the present disclosure contemplates using lyophilized ENPP1 formulations in treating or preventing diseases or conditions that are associated with abnormal activity of an ENPP1 polypeptide.

In one aspect, the ENPP1 formulations of the present disclosure are lyophilized. During lyophilization, ENPP1 polypeptide is converted from being in an aqueous phase to being in an amorphous solid phase, which is thought to protect the protein from chemical and/or conformational instability. Lyophilization is carried out using techniques common in the art and the lyophilized formulations are optimized for stability, shelf-life, and decreased levels of high molecular weight (HMW) species and aggregates. Tang et al., Pharm Res. 21:191-200, (2004) and Chang et al., Pharm Res. 13:243-9 (1996). The lyophilized ENPP1 formulations disclosed aid in stabilizing the protein against the stresses of manufacturing, shipping and storage. The excipients and additives used in the lyophilized formulations are integral components of a formulation, and therefore need to be safe and well tolerated by patients. For protein drugs, the choice of excipients and additives is particularly important because they can affect both efficacy and immunogenicity of the drug. Excipients and additives are also useful in reducing viscosity of high concentration ENPP1 polypeptide formulations in order to enable their delivery and enhance patient convenience. The formulation excipients and additives disclosed herein provide stability against these stresses. Common excipients are known in the art and can be found in Powell et al., Compendium of Excipients fir Parenteral Formulations (1998), PDA J. Pharm. Sci. Technology, 52:238-311.

In certain embodiments the ENPP1 formulations comprise stabilizers. These stabilizers can be classified on the basis of the mechanisms by which they stabilize proteins against various chemical and physical stresses. Some stabilizers are used to alleviate the effects of a specific stress or to regulate a particular susceptibility of a specific protein. Other stabilizers have more general effects on the physical and covalent stabilities of proteins. Given the teachings and guidance provided herein, those skilled in the art will know what amount or range of stabilizer can be included in any particular formulation to achieve an ENPP1 formulation of the disclosure that is likely to promote retention and stability of the ENPP1 polypeptide.

In certain embodiments, the ENPP1 formulations disclosed herein comprise bulking agents. Such bulking agents are included for the purpose of long-term stabilization, bulking up the solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity, or enhancing solubility.

The ENPP1 formulations disclosed herein comprise buffering agents, stabilizing agents, surfactants, sugars, salts and amino acids, which are described in greater detail below. A person having ordinary skill in the art would recognize that the concentrations of the excipients described herein share an interdependency within a particular formulation. By way of example, the concentration of a bulking agent is, in one aspect, lowered where, e.g., there is a high protein concentration or where, e.g., there is a high stabilizing agent concentration. In addition, a person having ordinary skill in the art would recognize that, in order to maintain the isotonicity of a particular formulation in which there is no bulking agent, the concentration of a stabilizing agent could be increased accordingly (i.e., a “tonicifying” amount of stabilizer would be used. Excipients and other additives are added to impart or enhance manufacturability and/or final product quality, such as the stability and delivery of a drug product (e.g., protein). The ENPP1 formulations disclosed herein comprise suitable excipients that enhance suitable stability, safety, and marketability.

In certain embodiments the lyophilized ENPP1 formulation comprises at least of one or more of a buffer, a bulking agent, stabilizer, and/or a surfactant. In some embodiments, the surfactant is selected to help reducing formation of HMW species, in cases where aggregation during the lyophilization step or during reconstitution becomes an issue. An appropriate buffering agent is included to maintain the formulation within stable zones of pH during manufacturing (e.g., dilution, sterile filtration, filling, etc.) and after reconstitution of the lyophilized product. The table below provides certain excipient components useful for lyophilized protein formulations:

TABLE 1
Excipient components of lyophilized protein formulations

| Excipient | |
|---|---|
| component | Function in lyophilized formulation |
| Buffer | Maintain pH of formulation during processing and |
| | upon reconstitution |
| Stabilizer | Stabilizers include cryo and lyoprotectants, such |
| | as polyols, sugars and polysaccharides |
| | Cryoprotectants protect proteins from freezing |
| | stresses |
| | Lyoprotectants stabilize proteins in the freeze- |
| | dried state |
| Bulking agent | Used to enhance product elegance and to prevent |
| | blowout |
| | Provides structural strength to the lyo cake |
| | Examples include mannitol and sucrose |
| Surfactant | Employed if aggregation during the lyophilization |
| | process is an issue |
| | May serve to reduce reconstitution times |
| | Examples include polysorbate 20 and 80 |
| Anti-oxidant | Oxidation reactions in the lyo cake are greatly |
| | retarded |
| Metal ions/ | May be included if a specific metal ion is |
| chelating | included only as a co-factor or where the metal |
| agent | is required for protease activity |
| Preservative | For multi-dose formulations only |
| | Provides protection against microbial growth in |
| | formulation |
| | Is usually included in the reconstitution diluent |
| | (e.g., bWFI) |

### (a) Buffers and Buffering Agents

Typically, the stability of a pharmacologically active polypeptide formulation is observed to be maximal in a narrow pH range. This pH range of optimal stability needs to be identified early during pre-formulation studies. Several approaches, such as accelerated stability studies and calorimetric screening studies, are useful in this endeavor (Remmele R. L. Jr., et al., Biochemistry, 38 (16): 5241-7 (1999)). Once a formulation is finalized, the protein must be manufactured and maintained throughout its shelf-life. Hence, buffering agents are almost always employed to control pH in the formulation.

Several factors must be considered when choosing a buffer. First and foremost, the buffer species and its concentration must be defined based on its pKa and the desired formulation pH. Equally important is to ensure that the buffer is compatible with the protein and other formulation excipients and does not catalyze any degradation reactions. A third important aspect to be considered is the sensation of stinging and irritation the buffer may induce upon administration. The potential for stinging and irritation is greater for drugs that are administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored. One has to be particularly careful about potassium ions administered in the form of the potassium phosphate buffer, which can induce cardiovascular effects in a patient (Hollander-Rodriguez J C, et al., Am. Fam. Physician., 73 (2): 283-90 (2006)).

Buffers for lyophilized formulations need additional consideration. Some buffers such as sodium phosphate can crystallize out of the protein amorphous phase during freezing resulting in shifts in pH. Other common buffers such as acetate and imidazole may sublime or evaporate during the lyophilization process, thereby shifting the pH of formulation during lyophilization or after reconstitution.

In certain embodiments, the exemplary buffering agents used to buffer the ENPP1 formulation as set forth herein include, but are not limited to organic acids, succinate, phosphate, acetate, citrate, Tris, HEPES, and amino acids or mixtures of amino acids, including, but not limited to aspartate, histidine, arginine and glycine. In certain embodiments the buffering agents are succinate, citrate, and phosphate. In one embodiment, the buffer system present in the ENPP1 formulation is selected to be physiologically compatible and to maintain a desired pH of the pharmaceutical formulation. In another embodiment, the pH of the solution is between pH 2.0 and pH 12.0. For example, in various embodiments the pH of the solution may be 5.5, 5.7, 6.0, 6.3, 6.5, 6.7, 7.0, 7.3, 7.5, 7.7, 8.0, 8.3, 8.5, 8.7, 9.0, 9.3, 9.5, 9.7, or 10.0.

The pH buffering compound may be present in any amount suitable to maintain the pH of the ENPP1 formulation at a predetermined level. When appropriately low levels of buffer are used, crystallization and pH shifts may be avoided. In one embodiment, the pH buffering concentration is between 0.1 mM and 500 mM (1 M). For example, it is contemplated that the pH buffering agent is at least 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the buffering agent maintains a pH range from pH 6-7 when reconstituted in solution. In some embodiments, the buffering agent maintains a pH range from pH 7-8 when reconstituted in solution.

Exemplary pH buffering agents used to buffer the ENPP1 formulation as set out herein include, but are not limited to, organic acids, succinate, phosphate, acetate, citrate, Tris, HEPES, and amino acids or mixtures of amino acids. In some embodiments, the buffering agent decreases formation of high molecular weight species by at least 10%, 15%, 20%, 25%, 30%, 35%, 40%, 45%, 50%.

### (b) Stabilizers and Bulking Agents

In one embodiment of the present pharmaceutical formulations, a stabilizer (or a combination of stabilizers) is added to prevent or reduce storage-induced aggregation and chemical degradation. A hazy or turbid solution upon reconstitution normally indicates that the protein has precipitated or at least aggregated. The term “stabilizer” means an excipient capable of preventing aggregation, or chemical degradation (for example, autolysis, deamidation, oxidation, etc.). In certain embodiments, the ENPP1 formulation provided herein include stabilizers such as, but are not limited to, sucrose, trehalose, mannose, maltose, lactose, glucose, raffinose, cellobiose, gentiobiose, isomaltose, arabinose, glucosamine, fructose, mannitol, sorbitol, poly-hydroxy compounds, including polysaccharides such as dextran, starch, hydroxyethyl starch, cyclodextrins, N-methyl pyrollidene, cellulose and hyaluronic acid [Carpenter et al., Develop. Biol. Standard 74:225, (1991)]. In one embodiment of the disclosure, sucrose and mannitol are used as a stabilizing agent in the ENPP1 formulations disclosed herein. In another embodiment of the disclosure, sucrose is used as a stabilizing agent.

In certain embodiments, the ENPP1 formulation comprises a stabilizer in a concentration of about 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. Likewise, in certain embodiments of the disclosure, the stabilizer is incorporated in a concentration of about 0.005, 0.01, 0.02, 0.03, 0.05, 0.06, 0.07, 0.08, 0.09, 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, or 20% w/v.

In other embodiments, the ENPP1 formulations also include appropriate amounts of bulking and osmolarity regulating agents. In various embodiments of the disclosure, bulking agents include, for example polymers such as dextran, polyvinylpyrolidone, carboxymethylcellulose, lactose, sorbitol, trehalose, or xylitol. The bulking agent is incorporated, in various embodiments of the disclosure, in a concentration of about 0.1, 0.5, 0.7, 0.8, 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM.

### (c) Surfactants

In certain embodiments, the ENPP1 formulations disclosed herein may additionally include surfactants. Surfactants are commonly used in protein formulations to prevent surface-induced degradation. Surfactants are amphipathic molecules with the capability of out-competing proteins for interfacial positions (and/or promote proper refolding of a structurally altered protein molecule). Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants contemplated herein include, without limitation, fatty acid esters of sorbitan polyethoxylates, i.e., polysorbate 20 and polysorbate 80. The two differ only in the length of the aliphatic chain that imparts hydrophobic character to the molecules, C-12 and C-18, respectively. Accordingly, polysorbate-80 is more surface-active and has a lower critical micellar concentration than polysorbate-20.

Detergents can also affect the thermodynamic conformational stability of proteins. Non-ionic surfactants are generally useful in protein stabilization. Ionic surfactants (detergents) normally destabilize proteins. Here again, the effects of a given detergent excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e. increasing the exposure of hydrophobic portions of the protein molecule in complement to binding polysorbate). Alternatively, if the protein native state exhibits some hydrophobic surfaces, detergent binding to the native state may stabilize that conformation. Another aspect of polysorbates is that they are inherently susceptible to oxidative degradation. Often, as raw materials, they contain sufficient quantities of peroxides to cause oxidation of protein residue side chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon during freezing and drying [Chang, B, J. Pharm. Sci. 85:1325, (1996)]. Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, 60, 65 and 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. Compositions comprising these surfactants, either individually or as a mixture in different ratios, are therefore further provided. In one embodiment of the present disclosure, the surfactant is TWEEN-80. In the present formulations, the surfactant is incorporated in a concentration of about 0.01 to about 0.5 g/L. In formulations provided herein, in various embodiments, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 g/L. Likewise, in certain embodiments of the disclosure, the surfactant is incorporated in a concentration of about 0.001, 0.002, 0.003, 0.004, 0.005, 0.01, 0.02, 0.03, 0.05, 0.06, 0.07, 0.08, 0.09, 0.1, 0.5, 0.7, 0.8, 0.9, or 1.0% w/v.

### (d) Salts

In certain embodiments, the ENPP1 formulations disclosed herein include salts. Salts are often added to increase the ionic strength of the formulation, which can be important for protein solubility, physical stability, and isotonicity. Salts can affect the physical stability of proteins in a variety of ways. Ions can stabilize the native state of proteins by binding to charged residues on the protein's surface. Alternatively, salts can stabilize the denatured state by binding to peptide groups along the protein backbone (—CONH—). Salts can also stabilize the protein native conformation by shielding repulsive electrostatic interactions between residues within a protein molecule. Salts in protein formulations can also shield attractive electrostatic interactions between protein molecules that can lead to protein aggregation and insolubility. Salts (i.e., electrolytes) sometimes make it more difficult to freeze dry the formulation. For this reason, only sufficient salt to maintain protein structural stability should be included in the formulation, and normally this level of electrolyte is very low.

In certain embodiments, the ENPP1 formulations disclosed herein may include salts such as for example sodium chloride (NaCl), Calcium chloride (CaCl₂)). Zinc chloride (ZnCl₂), and/or Magnesium chloride (MgCl₂) salts. In certain embodiments, the ENPP1 formulations disclosed herein have a salt concentration of the formulations is between 0.0 (i.e., no salt), 0.001, 0.002, 0.003, 0.004, 0.005, 0.006, 0.007, 0.008, 0.009, 0.010, 0.011, 0.012, 0.013, 0.014, 0.015, 0.020, 0.050, 0.080, 0.1, 1, 10, 20, 30, 40, 50, 80, 100, 120, 150, 200, 300, and 500 mM. In one embodiment of the disclosure, 0.0 mM NaCl (i.e., no NaCl) is included in the formulation.

### (e) Amino Acids

Amino acids have found versatile use in protein formulations as buffers, bulking agents, stabilizers and antioxidants. Thus, in one aspect the ENPP1 formulations disclosed herein include amino acids such as for example glycine, arginine, histidine, alanine, proline, serine, and glutamic acid. These amino acids often provide multiple benefits to the polypeptide formulations. Histidine is commonly found in marketed protein formulations, and this amino acid provides an alternative to citrate, a buffer known to sting upon injection. Interestingly, histidine has also been reported to have a stabilizing effect, with respect to aggregation when used at high concentrations in both liquid and lyophilized presentations (Chen B, et al., Pharm Res., 20 (12): 1952-60 (2003)). Histidine was also observed by others to reduce the viscosity of a high protein concentration formulation. In other aspects, formulations are provided that include one or more of the amino acids glycine, arginine and alanine, and have been shown to stabilize proteins by the mechanism of preferential exclusion. Glycine is also a commonly used bulking agent in lyophilized formulations. Arginine has been shown to be an effective agent in inhibiting aggregation and has been used in both liquid and lyophilized formulations. In the ENPP1 formulations provided, the amino acid concentration is between 0.1, 1, 10, 20, 30, 40, 50, 80, 100, 120, 150, 200, 300, and 500 mM. In one embodiment of the present disclosure, the amino acid is glycine.

### (f) Antioxidants

Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination. The disclosure therefore contemplates the use of the pharmaceutical antioxidants including, without limitation, reducing agents, oxygen/free-radical scavengers, or chelating agents. Antioxidants in therapeutic protein formulations are, in one aspect, water-soluble and remain active throughout the product shelf-life. Reducing agents and oxygen/free-radical scavengers work by ablating active oxygen species in solution. In various embodiments of the formulations provided herein, the antioxidant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 mg/mL.

### (g) Metal Ions

In general, transition metal ions are undesired in protein formulations because they can catalyze physical and chemical degradation reactions in proteins. However, specific metal ions are included in formulations when they are cofactors to proteins and in suspension formulations of proteins where they form coordination complexes (e.g., zinc suspension of insulin). Recently, the use of magnesium ions (10-120 mM) has been proposed to inhibit the isomerization of aspartic acid to isoaspartic acid (WO 2004039337).

### (h) Preservatives

Preservatives are necessary when developing multi-use parenteral formulations that involve more than one extraction from the same container. Their primary function is to inhibit microbial growth and ensure product sterility throughout the shelf-life or term of use of the drug product. Commonly used preservatives include, without limitation, benzyl alcohol, phenol and m-cresol. Although preservatives have a long history of use, the development of protein formulations that includes preservatives can be challenging. Preservatives almost always have a destabilizing effect (aggregation) on proteins, and this has become a major factor in limiting their use in multi-dose protein formulations (Roy S, et al., J Pharm Sci., 94 (2): 382-96 (2005)). When practical, preservatives should be included in the diluent formulation and not included in the formulation to be freeze dried.

In certain embodiments, the soluble ENPP1 polypeptide is formulated as a lyophilized polypeptide formulation comprising a therapeutic amount of a soluble ENPP1 polypeptide disclosed herein, whereby the lyophilized polypeptide formulation is reconstitutable to a solution in liquid form. In some embodiments, the lyophilized polypeptide formulation is reconstituted in a sterile injectable solution. In some embodiments, the lyophilized polypeptide formulation is reconstituted in a reconstitution solution. In some embodiments, the reconstitution solution comprises a pharmaceutically acceptable carrier and/or additive. In some embodiments, the pharmaceutically acceptable carrier is selected from saline solution, purified water, or sterile water for injection. In some embodiments, the formulation is completely reconstituted within a period of less than 100 seconds, 80 seconds, 70 seconds, 69 seconds, 68 seconds, 67 seconds, 66 seconds, 65 seconds, 64 seconds, 63 seconds, 62 seconds, 61 seconds, or 60 seconds.

In certain embodiments, the lyophilized polypeptide formulation comprises an ENPP1 polypeptide. In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO:1. In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of a polypeptide with the amino acid sequence of SEQ ID NO:2. In some embodiments, the ENPP1 polypeptide is a fusion protein. In some embodiments, the ENPP1 polypeptide is a fusion protein further comprising an Fc domain of an immunoglobulin. In some embodiments, the Fc domain of the immunoglobulin is an Fc domain of an IgG1 immunoglobulin. In some embodiments, the ENPP1 polypeptide is a fusion protein comprising a soluble ENPP1 polypeptide domain and one or more heterologous protein portions. In some embodiments, the heterologous protein portion increases the circulating half-life of the soluble ENPP1 polypeptide in a mammal. In some embodiments, the heterologous protein portion comprises an Fc domain. In some embodiments, the Fc domain comprises a polypeptide that comprises, consists essentially of, or consists of an amino acid sequence that is at least 75%, 80%, 85%, 90%, 91%, 92%, 93%, 94%, 95%, 96%, 97%, 98%, 99%, or more identical to the amino acid sequence of SEQ ID NO:12. In some embodiments, the ENPP1 polypeptide further comprises a heterologous moiety. In some embodiments, the heterologous moiety is selected from the group consisting of a glycosylated amino acid, a PEGylated amino acid, a farnesylated amino acid, an acetylated amino acid, a biotinylated amino acid, and a lipid moiety.

In certain embodiments, the lyophilized polypeptide formulation comprises a buffering agent. In certain embodiments, the buffering agent maintains a pH in the range of pH 5.0 to pH 8.0, in the range of about pH 5.5 to pH 7.5, in the range of about pH 6.0 to pH 7.0, in the range of about pH 6.0 to pH 6.5. In some embodiments the buffering agent maintains a pH of pH 6.5+/−0.5. In a preferred embodiment, the pH is pH 6.3. In certain embodiments, the buffering agent is selected from the group consisting of: succinate, citrate, bicarbonate, phosphate, tris, or glycylglycine. In certain preferred embodiments, the buffering agent is succinate, citrate, or phosphate. In a preferred embodiment, the buffering agent is citrate. The buffering agent may comprise a concentration ranging from 5 mM to 100 mM when the lyophilized polypeptide formulation is reconstituted in solution. In certain embodiments, the buffering agent is present at a concentration ranging from 10 mM to 50 mM, or 15 mM to 25 mM. In certain preferred embodiments, the buffering agent is present at a concentration of 20 mM. The buffering agent may impart increased stability of the ENPP1 polypeptide in both lyophilized and reconstituted form. In certain embodiments, the buffering agent may be selected to facilitate increased onset temperature of aggregate formation. In some embodiments, the buffering agent increases the onset temperature of aggregate formation by at least 1° C., 2° C., 3° C., 4° C., 5° C., 6° C., 7° C., 8° C., 9° C., or 10° C. In certain embodiments, the buffering agent may decrease formation of high molecular weight species. In some embodiments, the buffering agent decreases formation of high molecular weight species by at least 2%, 3%, 4%, 5%, 10%, 15%, 20%, 30%, 40%, 50% 60%, 70%, 80%, 90%, or 100%. In some embodiments, the buffering agent decreases formation of high molecular weight species by at least 10% to 100%, 20% to 80%, 30% to 70%, or 40% to 60%. In some embodiments, the buffering agent decreases formation of high molecular weight species by at least 50%.

Lyophilized polypeptide formulations of the present disclosure may comprise one or more pharmaceutically acceptable additives and/or stabilizing agents. In certain embodiments, the pharmaceutically acceptable additive comprises amino acids, salts, sugars, and/or polyols. In some embodiments, the pharmaceutically acceptable additive is arginine, proline, and/or glycine. In preferred embodiments, the pharmaceutically acceptable additive is a stabilizing agent such as sucrose and/or mannitol. In certain embodiments, the one or more pharmaceutically acceptable additives comprise a concentration ranging from 50 mM to 300 mM when reconstituted in solution. In some embodiments, the one or more pharmaceutically acceptable additives comprise a concentration ranging from 50 mM to 100 mM, 60 mM to 90 mM, or 75 mM to 85 mM, when reconstituted in solution. In some embodiments, the one or more pharmaceutically acceptable additives comprise a concentration within +/−5 mM of 50 mM, 60 mM, 70 mM, 80 mM, 82 mM, 84 mM, 86 mM, 88 mM, 90 mM, 100 mM, 110 mM, 120 mM, 130 mM, 140 mM, 150 mM, 160 mM, 170 mM, 171 mM, 172 mM, 173 mM, 174 mM, 175 mM, 176 mM, 177 mM, 178 mM, 179 mM, 180 mM, 190 mM, 200 mM, 210 mM, 220 mM, 230 mM, 240 mM, 250 mM, 260 mM, 261 mM, 262 mM, 263 mM, 264 mM, 265 mM, 266 mM, 267 mM, 268 mM, 269 mM, 270 mM, 280 mM, 290 mM, 300 mM, when reconstituted in solution. The pharmaceutically acceptable additive and/or stabilizing agent may impart increased stability of the ENPP1 polypeptide in both lyophilized and reconstituted form. In certain embodiments, the pharmaceutically acceptable additive and/or stabilizing agent decreases formation of high molecular weight species by at least 2%, 3%, 4%, 5%, 10%, 15%, 20%, 30%, 40%, 50% 60%, 70%, 80%, 90%, or 100%. In some embodiments, the pharmaceutically acceptable additive and/or stabilizing agent decreases formation of high molecular weight species by at least 10% to 100%, 20% to 80%, 30% to 70%, or 40% to 60%. In some embodiments, the pharmaceutically acceptable additive and/or stabilizing agent decreases formation of high molecular weight species by at least 50%.

ENPP1 polypeptides of the present disclosure comprise cofactors coordinated within the ENPP1 nuclease-like and SMB-like domains (see, e.g., Kato K. et al., Proc Natl Acad Sci USA. 2012; 109 (42): 16876-81). In certain embodiments, the lyophilized polypeptide formulations of the present disclosure may comprise one or more ENPP1 polypeptide cofactors. In some embodiments, the ENPP1 polypeptide cofactor comprises calcium, zinc, and/or adenosine monophosphate. In some embodiments, the ENPP1 polypeptide cofactor is CaCl₂, CaSO₄, ZnCl₂, ZnSO₄, and/or adenosine monophosphate. In some embodiments, the ENPP1 polypeptide cofactor is CaCl₂) and/or adenosine monophosphate. In a preferred embodiment, the cofactor ENPP1 polypeptide cofactor is CaCl₂. In some embodiments, the ENPP1 polypeptide cofactor comprises a concentration ranging from 1 mM to 10 mM, 1 mM to 5 mM, or 1 mM to 3 mM when reconstituted in solution. In a preferred embodiment, the ENPP1 polypeptide cofactor comprises a concentration within +/−1 mM of 2 mM. In a further preferred embodiment, the ENPP1 polypeptide cofactor comprises a concentration of 2 mM.

In some embodiments, the compositions and formulations comprise a surfactant. In some embodiments, the surfactant comprises, a polysorbate, poloxamer, triton, sodium dodecyl sulfate, sodium laurel sulfate, sodium octyl glycoside, laurylsulfobetaine, myristyl-sulfobetaine, linoleyl-sulfobetaine, stearyl-sulfobetaine, laurylsarcosine, myristyl-sarcosine, linoleyl-sarcosine, stearyl-sarcosine, linoleyl-betaine, myristyl-betaine, cetyl-betaine, lauroam idopropyl-betaine, cocam idopropyl-betaine, linoleamidopropyl-betaine, myristam idopropyl-betaine, palm idopropyl-betaine, isostearam idopropyl-betaine, myristam idopropyl-dimethylamine, palm idopropyldimethylamine, isostearam idopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. The surfactant can be, for example without limitation, polysorbate 20, polysorbate 21, polysorbate 40, polysorbate 60, polysorbate 61, polysorbate 65, polysorbate 80, polysorbate 81, polysorbate 85, polysorbate 188, PEG3350, and mixtures thereof. In preferred embodiments, the surfactant is polysorbate 20 (PS20), polysorbate 80 (PS80), or polysorbate 188 (PS188). In a preferred embodiment, the surfactant is polysorbate 20 (PS20).

The concentration of the surfactant may be expressed as a percentage (w/v). For lyophilized formulations, the % (w/v) concentration represents the surfactant concentration when reconstituted in solution. The concentration of the surfactant generally ranges from about 0.001% (w/v) to 1% (w/v). In some embodiments, the concentration of the surfactant ranges from 0.01% to 0.5% (w/v), 0.015% to 0.25% (w/v), 0.02% to 0.1% (w/v). In a preferred embodiment, the concentration of the surfactant ranges from 0.02% to 0.1% (w/v).

Lyophilized polypeptide formulations of the present disclosure may exhibit increased stability over time. In certain embodiments, lyophilized polypeptide formulations of the present disclosure exhibit long term stability at −80° C. to 40° C. In some embodiments, the formulation has a shelf life of at least 3, 6, 12, 24, 36, 48, or 60 months. In some embodiments, the reconstituted formulation has a shelf life of at least 1, 2, 3, 4, 5, 6, 12, 18, 24, 48, or 60 hours.

In some embodiments, the reconstituted formulation is stable in concentrations ranging from 1 mg/ml to 50 mg/ml, 2 mg/ml to 45 mg/ml, 5 mg/ml to 40 mg/ml, 10-30 mg/ml, 15-20 mg/ml, for at least 8 hours at RT. In some embodiments, the reconstituted formulation is stable in concentrations ranging from 1 mg/ml to 50 mg/ml, 2 mg/ml to 45 mg/ml, 5 mg/ml to 40 mg/ml, 10-30 mg/ml, 15-20 mg/ml, for at least 24 hours at 5° C.

Lyophilized polypeptide formulations disclosed herein may be administered in reconstituted form. Routes of administration for reconstituted lyophilized polypeptide formulations disclosed herein include inhalational, oral, nasal, rectal, parenteral, sublingual, transdermal, transmucosal (e.g., sublingual, lingual, (trans) buccal, (trans) urethral, vaginal (e.g., trans- and perivaginally), intranasal, and (trans) rectal, intravesical, intrapulmonary, intraduodenal, intragastrical, intrathecal, subcutaneous, intramuscular, intradermal, intra-arterial, intravenous, intrabronchial, inhalation (e.g., aerosol), ophthalmic, pulmonary, and topical administration. In a preferred embodiment, the reconstituted formulation is administered parenterally. In some embodiments, the reconstituted formulation is administered via subcutaneous injection, intravenous injection, intradermal injection, intramuscular injection. In some embodiments, the reconstituted formulation in self-administered. In some embodiments, the lyophilized formulation comprises a therapeutically effective dose. In some embodiments, the formulation is administered several times daily, every two days, three days, one week, or one month. In some embodiments, a second dosage of the formulation is administered after a suitable time interval of at least after two days, after four days, after a week, or after a month.

In certain embodiments, the disclosure provides kits comprising a lyophilized polypeptide formulation. In some embodiments, the kit comprises a lyophilized polypeptide which may be reconstituted in a sterile injectable solution (or sterile water) prior to use. In some embodiments, the kit comprises a one or more vials. In some embodiments, the kit comprises one or more vials comprising the lyophilized polypeptide formulation. In some embodiments, the kit comprises an injectable device comprising a vial, reconstitution solution, a syringe, and/or a pre-filled syringe. In some embodiments, transfer of the reconstitution solution to the vial reconstitutes the lyophilized polypeptide in a sterile injectable solution. In some embodiments, the lyophilized polypeptide is reconstituted in a sterile injectable solution. In some embodiments, the lyophilized polypeptide is reconstituted in a sterile injectable solution (or sterile water) prior to use. In some embodiments, the injectable device is used to administer the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose.

The lyophilized polypeptide formulations disclosed herein may be used in methods of treating, reversing, or preventing progression of diseases associated with an ENPP1 Deficiency as disclosed herein. In some embodiments, the formulation is for use in methods of treating, reversing, or preventing progression of ossification of the posterior longitudinal ligament (OPLL) in a subject in need thereof. In some embodiments, the formulation is for use in methods of treating, reverting, or preventing progression of hypophosphatemic rickets in a subject in need thereof. In some embodiments, the formulation is for use in methods of treating, reversing, or preventing progression of ABCC6 Deficiency (e.g., as Pseudoxanthoma Elasticum (PXE)) in a subject in need thereof. In some embodiments, the formulation is for use in methods of reducing or preventing progression of age-related hardening of arteries in a subject in need thereof. In some embodiments, the formulation is for use in treating, reversing, or preventing progression of calcification of atherosclerotic plaques in vascular arteries in a subject in need thereof. In some embodiments, the formulation is for use in methods of treating, reversing, or preventing progression of osteoarthritis in a subject in need thereof. In some embodiments, the formulation is for use in methods of treating, reversing, or preventing progression of hardening of arteries due to progeria in a subject in need thereof. In some embodiments, the formulation is for use in methods of treating, reversing, or preventing progression of calcification of atherosclerotic plaques in vascular arteries in a subject in need thereof. In some embodiments, the formulation is for use in methods of treating, reversing, or preventing progression of osteoarthritis in a subject in need thereof. In some embodiments, the formulation is for use in methods of treating, reversing, or preventing progression of hardening of arteries due to progeria in a subject in need thereof. In some embodiments, the formulation is for use in methods of treating, reversing, or preventing progression of X-linked hypophosphatemic rickets (XLH), hereditary hypophosphatemic rickets (HHRH), hypophosphatemic bone disease (HBD), autosomal dominant hypophosphatemic rickets (ADHR), and/or and autosomal recessive hypophosphatemic rickets in a subject in need thereof. In some embodiments, the formulation is for use in methods of treating, reversing, or preventing progression of age-related osteopenia in a subject in need thereof. In some embodiments, the formulation is for use in methods of treating, reversing, or preventing progression of ankylosing spondylitis in a subject in need thereof. In some embodiments, the formulation is for use in methods of treating, reversing, or preventing progression of strokes in pediatric sickle cell anemia in a subject in need

The formulation disclosed herein are lyophilized using techniques that are well known in the art. Further information on lyophilization may be found in Carpenter, J. F. and Chang, B. S., Lyophilization of Protein Pharmaceuticals, Biotechnology and Biopharmaceutical Manufacturing, Processing and Preservation, K. E. Avis and V. L. Wu, eds. (Buffalo Grove, Interpharm Press, Inc.), pp. 199 264 (1996), U.S. Pat. Nos. 7,247,707; 7,087,723; and 6,586,573.

### 3. ENPP1 Polypeptides

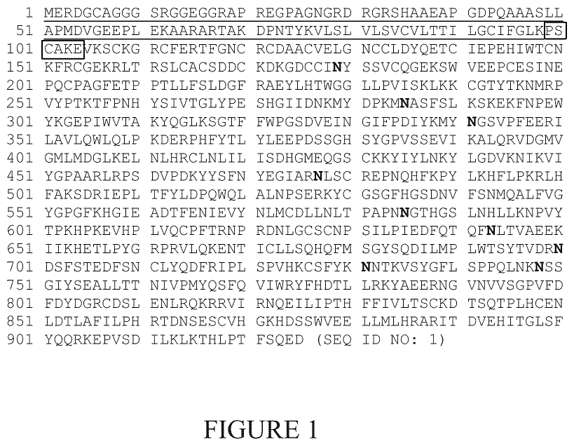

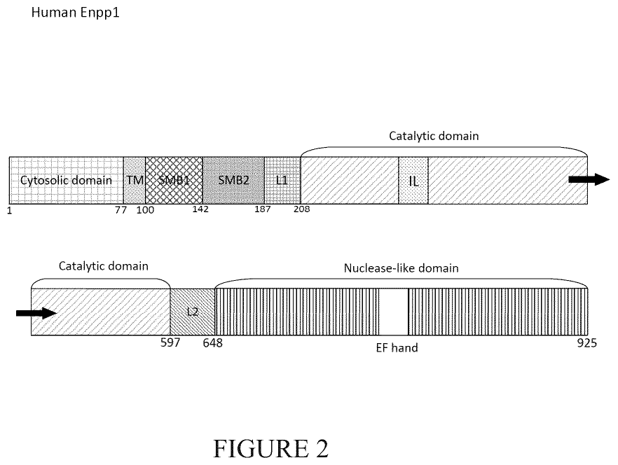

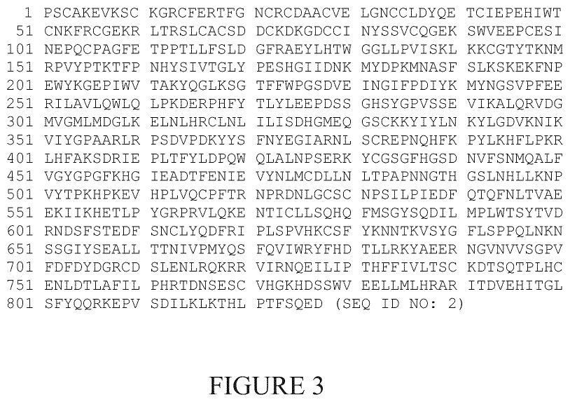

In certain aspects, the present disclosure relates lyophilized formulations of ENPP1 polypeptides and uses thereof. ENPP1 polypeptides disclosed herein include naturally occurring polypeptides of the ENPP1 family as well as any variants thereof (including mutants, fragments, fusions, and peptidomimetic forms) that retain a biological activity. The terms “ENPP1” or “ENPP1 polypeptide” refers to ectonucleotide pyrophosphatase/phosphodiesterase 1 proteins (NPP1/ENPP1/PC-1) and ENPP1-related proteins, derived from any species. ENPP1 protein comprises a type II transmembrane glycoprotein that forms a homodimer. Each monomer of the ENPP1 protein comprises a short intracellular N-terminal domain involved in targeting to the plasma membrane, a transmembrane domain, and a large extracellular region comprising several domains. The large extracellular region comprises SMB1 and SMB2 domains, which have been reported to take part in ENPP1 dimerization (R. Gijsbers, H. et al., Biochem. J. 371; 2003:321-330). Specifically, the SMB domains contain eight cysteine residues, each arranged in four disulphide bonds, and have been shown to mediate ENPP1 homodimerization through covalent cystine inter- and intramolecular bonds. The protein cleaves a variety of substrates, including phosphodiester bonds of nucleotides and nucleotide sugars and pyrophosphate bonds of nucleotides and nucleotide sugars. ENPP1 protein functions to hydrolyze nucleoside 5′ triphosphatase to either corresponding monophosphates and also hydrolyzes diadenosine polyphosphates. ENPP1 proteins play a role in purinergic signaling which is involved in the regulation of cardiovascular, neurological, immunological, musculoskeletal, hormonal, and hematological functions. An exemplary amino acid sequence of the human ENPP1 precursor protein (NCBI accession NP_006199) is shown in FIG. 1 (SEQ ID NO: 1). The human ENPP1 precursor protein includes an endogenous ENPP1 signal peptide sequence at the ENPP1 N-terminus. Numbering of amino acids for all ENPP1-related polypeptides described herein is based on the numbering of the human ENPP1 precursor protein sequence provided in FIG. 1 unless specifically designated otherwise. In certain embodiments, the ENPP1 precursor protein further comprises an endogenous or heterologous signal peptide sequence. Upon proteolysis, the signal peptide sequence is cleaved from the ENPP1 precursor protein to provide the mature ENPP1 protein. See, e.g., Jansen S, et al. J Cell Sci. 2005; 118 (Pt 14):3081-9. Exemplary signal peptide sequences that can be used with the polypeptides disclosed herein include, but are not limited to, ENPP1 signal peptide sequence, ENPP2 signal peptide sequence, Azurocidin signal sequence, ENPP7 signal peptide sequence, and/or ENPP5 signal peptide sequence. The processed (mature) extracellular ENPP1 polypeptide sequence is shown in FIG. 3 (SEQ ID NO: 2).

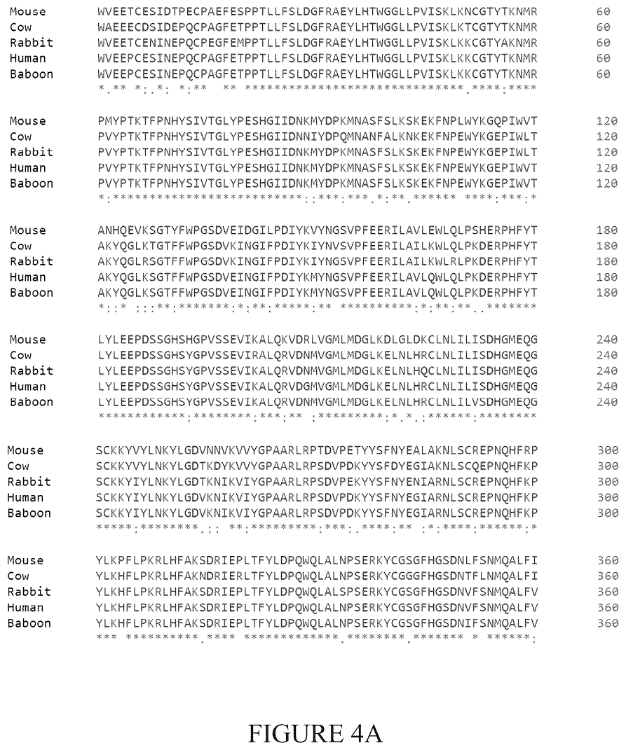

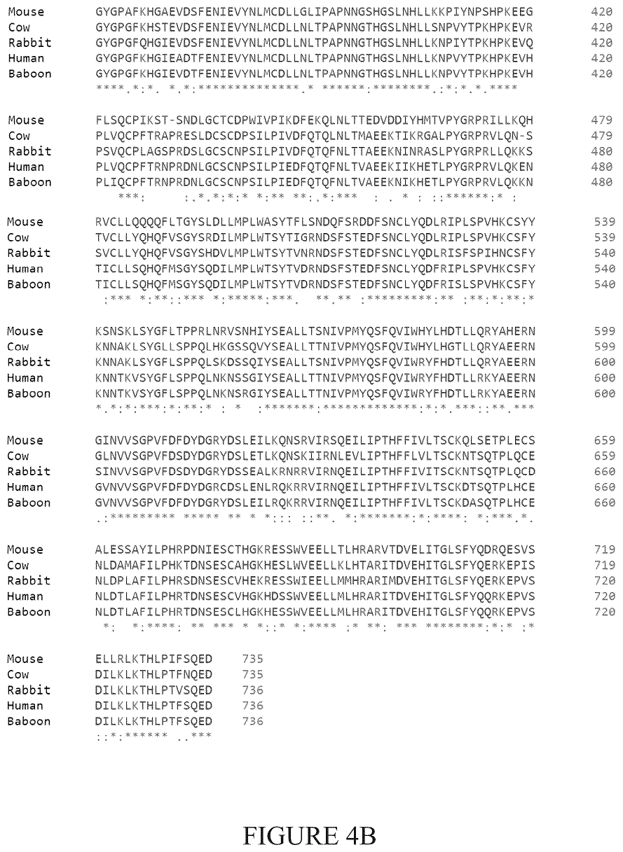

It is generally known in the art that ENPP1 is well-conserved among vertebrates, with large stretches of the extracellular domain substantially conserved. For example, FIG. 4A and FIG. 4B depict a multi-sequence alignment of a human ENPP1 extracellular domain compared to various ENPP1 orthologs. ENPP1 binding to various nucleotide triphosphates (e.g., ATP, UTP, GTP, TTP, and CTP), pNP-TMP, 3′,5′-CAMP, and 2′-3′-cGAMP is also highly conserved (see, e.g., Kato K. et al., Proc Natl Acad Sci USA. 2012; 109 (42): 16876-81 and Mackenzie N C, et al. Bone. 2012; 51 (5): 961-8). Accordingly, from these alignments, it is possible to predict key amino acid positions with the extracellular domain that are important for normal ENPP1 activities as well as to predict amino acid positions that are likely to be tolerant to substitution without significantly altering normal ENPP1 activities. Therefore, an active, human ENPP1 polypeptide useful in accordance with the presently disclosed compositions, may include one or more amino acids at corresponding positions from the sequence of another vertebrate ENPP1, or may include a residue that is similar to that in the human or other vertebrate sequences. Substitutions of one or more amino acids at corresponding positions may include conservative variations or substitutions that are not likely to change the shape of the polypeptide chain or alter normal ENPP1 activities. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine. For example, ENPP1 polypeptides include polypeptides derived from the sequence of any known ENPP1 polypeptide having a sequence at least about 80% identical to the sequence of an ENPP1 polypeptide, and preferably at least 85%, 90%, 91%, 92%, 93%, 94%, 95%, 96%, 97%, 98%, 99% or greater identity.

ENPP1 proteins have been characterized in the art in terms of structural and biological characteristics. In certain embodiments, soluble ENPP1 proteins disclosed herein comprise pyrophosphatase and/or phosphodiesterase activity. For instance, in some embodiments, the ENPP1 protein binds nucleotide triphosphates (e.g., ATP, UTP, GTP, TTP, and CTP), pNP-TMP, 3′,5′-CAMP, and 2′-3′-cGAMP; and converts nucleotide triphosphates into inorganic pyrophosphate [see, e.g., Kato K. et al., Proc Natl Acad Sci USA. 2012; 109 (42): 16876-81; Li L, et al. Nat Chem Biol. 2014; 10 (12): 1043-8; Jansen S, et al. Structure. 2012; 20 (11): 1948-59; and Onyedibe K I, et al. Molecules. 2019; 24 (22)].

As used herein, the terms “enzymatically active” or “biologically active” refer to ENPP1 polypeptides that exhibit pyrophosphatase and/or phosphodiesterase activity (e.g., is capable of binding and/or hydrolyzing ATP into AMP and PPi and/or AP3a into ATP). For example, the pyrophosphatase/phosphodiesterase domain of an ENPP1 protein hydrolyzes extracellular nucleotide triphosphates to produce inorganic pyrophosphates (PPi) and is generally soluble. This activity can be measured using a pNP-TMP assay as previously described (Saunders, et al., 2008, Mol. Cancer Ther. 7 (10): 3352-62; Albright, et al., 2015, Nat Comm. 6:10006). In certain embodiments, the soluble ENPP1 polypeptide has a k_{cat} value for the substrate ATP greater than or equal to about 3.4 (+0.4) s′¹ enzyme′¹, wherein the k_{cat} is determined by measuring the rate of hydrolysis of ATP for the polypeptide. In certain embodiments, the soluble ENPP1 polypeptide has a Ky value for the substrate ATP less than or equal to about 2 pM, wherein the K_{M} is determined by measuring the rate of hydrolysis of ATP for the polypeptide. In addition to the teachings herein, these references provide ample guidance for how to generate soluble ENPP1 proteins that retain one or more biological activities (e.g., conversion of nucleotides into inorganic pyrophosphate).

In one embodiment, the disclosure relates to ENPP1 polypeptides. As described herein, the term soluble ENPP1 polypeptide, includes any naturally occurring extracellular domain of an ENPP1 protein as well as any variants thereof (including mutants, fragments and peptidomimetic forms) that retain a biological activity (e.g., enzymatically active). Examples of soluble ENPP1 polypeptides include, for example, an ENPP1 extracellular domain (SEQ ID NO: 2) as shown in FIG. 3. In certain embodiments, the soluble ENPP1 polypeptides further comprise a signal sequence in addition to the extracellular domain of an ENPP1 polypeptide. Exemplary signal sequences include the native signal sequence of an ENPP1 polypeptide, or a signal sequence from another protein, such as a hENPP7 signal sequence. Examples of variant soluble ENPP1 polypeptides are provided in International Patent Application Publication Nos. WO 2012/125182, WO 2014/126965, WO 2016/187408, WO 2018/027024, and WO 2020/047520 which are incorporated herein by reference in their entirety.

In some embodiments, the ENPP1 polypeptide is a fusion protein comprising an ENPP1 polypeptide domain and one or more heterologous protein portions (i.e., polypeptide domains heterologous to ENPP1). An amino acid sequence is understood to be heterologous to ENPP1 if it is not uniquely found in the form of ENPP1 represented by SEQ ID NO: 1. In some embodiments, the heterologous protein portion comprises an Fc domain of an immunoglobulin. In some embodiments, the Fc domain of the immunoglobulin is an Fc domain of an IgG1 immunoglobulin. In certain embodiments, the soluble ENPP1 polypeptide is C-terminally fused to the Fc domain of human immunoglobulin 1 (IgG1), human immunoglobulin 2 (IgG2), human immunoglobulin 3 (IgG3), and/or human immunoglobulin 4 (IgG4). In other embodiments, the soluble ENPP1 polypeptide is N-terminally fused to the Fc domain of human immunoglobulin 1 (IgG1), human immunoglobulin 2 (IgG2), human immunoglobulin 3 (IgG3), and/or human immunoglobulin 4 (IgG4). In some embodiments, the presence of an Fc domain improves half-life, solubility, reduces immunogenicity, and increases the activity of the soluble ENPP1 polypeptide. In certain embodiments, portions of the native human IgG proteins (IgG1, IgG2, IgG3, and IgG4), may be used for the Fc portion (e.g., ENPP1-Fc). For instance, the present disclosure provides fusion proteins comprising ENPP1 fused to a polypeptide comprising a constant domain of an immunoglobulin, such as a CH1, CH2, or CH3 domain derived from human IgG1, IgG2, IgG3, and/or IgG4. The Fc fragment may comprise regions of the native IgG such as the hinge region (residues 216-230 of human IgG1, according to the Rabat numbering system), the entire second constant domain CH2 (residues 231-340), and the third constant domain CH3 (residues 341-447).

As used herein, the term “ENPP1-Fc construct” refers to a soluble form of ENPP1 (e.g., the extracellular domain of an ENPP1 polypeptide) recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

An example of an amino acid sequence that may be used for the Fc portion of human IgG1 (G1Fc) is SEQ ID NO: 12 (Table 2). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

In some embodiments, the heterologous protein portion comprises one or more domains selected from the group consisting of polyhistidine, FLAG tag, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6) fusion partners. As another example, a fusion domain may be selected so as to facilitate detection of the ENPP1 polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation.

In some embodiments, the ENPP1 fusion protein further comprises a linker positioned between the ENPP1 polypeptide domain and the one or more heterologous protein portions (e.g., an Fc immunoglobulin domain). In certain embodiments, the soluble ENPP1 polypeptide is directly or indirectly fused to the Fc domain. In some embodiments, the soluble ENPP1 fusion protein comprises a linker between the Fc domain and the ENPP1 polypeptide. In some embodiments, a linker can be an amino acid spacer including 1-200 amino acids. Suitable peptide spacers are known in the art, and include, for example, peptide linkers containing flexible amino acid residues such as glycine, alanine, and serine. In some embodiments, the linker comprises a polyglycine linker or a Gly-Ser linker. In some embodiments, a spacer can contain motifs, e.g., multiple or repeating motifs, of GGGA (SEQ ID NO: 21), GGGS (SEQ ID NO: 22), GGGG (SEQ ID NO: 23), GGGGA (SEQ ID NO: 24), GGGGS (SEQ ID NO: 25), GGGGG (SEQ ID NO: 26), GGAG (SEQ ID NO: 27), GGSG (SEQ ID NO: 28), AGGG (SEQ ID NO: 29), SGGGG (SEQ ID NO: 30), SGGG (SEQ ID NO: 31), GA (SEQ ID NO: 103), GS (SEQ ID NO: 104), GG (SEQ ID NO: 105), GGA (SEQ ID NO: 106), GGS (SEQ ID NO: 107), or GGG (SEQ ID NO: 108). In some embodiments, a spacer can contain 2 to 12 amino acids including motifs of GA or GS, e.g., GA, GS, GAGA (SEQ ID NO: 32), GSGS (SEQ ID NO: 33), GAGAGA (SEQ ID NO: 34), GSGSGS (SEQ ID NO: 35), GAGAGAGA (SEQ ID NO: 36), GSGSGSGS (SEQ ID NO: 37), GAGAGAGAGA (SEQ ID NO: 38), GSGSGSGSGS (SEQ ID NO: 39), GAGAGAGAGAGA (SEQ ID NO: 40), and GSGSGSGSGSGS (SEQ ID NO: 41). In some embodiments, a spacer can contain 3 to 12 amino acids including motifs of GGA or GGS, e.g., GGA, GGS, GGAGGA (SEQ ID NO: 42), GGSGGS (SEQ ID NO: 43), GGAGGAGGA (SEQ ID NO: 44), GGSGGSGGS (SEQ ID NO: 45), GGAGGAGGAGGA (SEQ ID NO: 46), and GGSGGSGGSGGS (SEQ ID NO: 47). In yet some embodiments, a spacer can contain 4 to 12 amino acids including motifs of GGAG (SEQ ID NO: 48), GGSG (SEQ ID NO: 49), e.g., GGAG (SEQ ID NO: 50), GGSG (SEQ ID NO: 51), GGAGGGAG (SEQ ID NO: 52), GGSGGGSG (SEQ ID NO: 53), GGAGGGAGGGAG (SEQ ID NO: 54), and GGSGGGSGGGSG (SEQ ID NO: 55). In some embodiments, a spacer can contain motifs of GGGGA (SEQ ID NO: 56) or GGGGS (SEQ ID NO: 57), e.g., GGGGAGGGGAGGGGA (SEQ ID NO: 58) and GGGGSGGGGSGGGGS (SEQ ID NO: 59). In some embodiments of the invention, an amino acid spacer between a heterologous protein portion (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a soluble ENPP1 polypeptide may be GGG, GGGA (SEQ ID NO: 21), GGGG (SEQ ID NO: 23), GGGAG (SEQ ID NO: 60), GGGAGG (SEQ ID NO: 61), or GGGAGGG (SEQ ID NO: 62).

In some embodiments, a spacer can also contain amino acids other than glycine, alanine, and serine, e.g., TGGGG (SEQ ID NO: 63), AAAL (SEQ ID NO: 64), AAAK (SEQ ID NO: 65), AAAR (SEQ ID NO: 66), EGKSSGSGSESKST (SEQ ID NO: 67), GSAGSAAGSGEF (SEQ ID NO: 68), AEAAAKEAAAKA (SEQ ID NO: 69), KESGSVSSEQLAQFRSLD (SEQ ID NO: 70), GENLYFQSGG (SEQ ID NO: 71), SACYCELS (SEQ ID NO: 72), RSIAT (SEQ ID NO: 73), RPACKIPNDLKQKVMNH (SEQ ID NO: 74), GGSAGGSGSGSSGGSSGASGTGTAGGTGSGSGTGSG (SEQ ID NO: 75), AAANSSIDLISVPVDSR (SEQ ID NO: 76), GGSGGGSEGGGSEGGGSEGGGSEGGGSEGGGSGGGS (SEQ ID NO: 77), (R)ₘ; m=0-15 (SEQ ID NO: 78), DSSSEEKFLRRIGRFG (SEQ ID NO: 79), EEEEEEEPRGDT (SEQ ID NO: 80), APWHLSSQYSRT (SEQ ID NO: 81), STLPIPHEFSRE (SEQ ID NO: 82), VTKHLNQISQSY (SEQ ID NO: 83), (E) m; m=1-15 (SEQ ID NO: 84), RSGSGGS (SEQ ID NO: 85), (D) m; m=1-15 (SEQ ID NO: 86), LVIMSLGLGLGLGLRK (SEQ ID NO: 87), VIMSLGLGLGLGLRK (SEQ ID NO: 88), IMSLGLGLGLGLRK (SEQ ID NO: 89), MSLGLGLGLGLRK (SEQ ID NO: 90), SLGLGLGLGLRK (SEQ ID NO: 91), LGLGLGLGLRK (SEQ ID NO: 92), GLGLGLGLRK (SEQ ID NO: 93), LGLGLGLRK (SEQ ID NO: 94), GLGLGLRK (SEQ ID NO: 95), LGLGLRK (SEQ ID NO: 96), GLGLRK (SEQ ID NO: 97), LGLRK (SEQ ID NO: 98), GLRK (SEQ ID NO: 99), (K)ₘ; m=1-15 (SEQ ID NO: 100), LIN (SEQ ID NO: 109), NSS (SEQ ID NO: 110), ESS (SEQ ID NO: 111), RQQ (SEQ ID NO: 112), KR (SEQ ID NO: 113), LRK (SEQ ID NO: 114), or RK (SEQ ID NO: 115). In some embodiments, a spacer can contain motifs, e.g., multiple or repeating motifs, of EAAAK (SEQ ID NO: 101). In some embodiments, a spacer can contain motifs, e.g., multiple or repeating motifs, of proline-rich sequences such as (XP)n, in which X may be any amino acid (e.g., A, K, or E) and n is from 1-5, and PAPAP (SEQ ID NO: 102).

```
ENPP2 signal sequence
SEQ. ID NO: 3
Leu Phe Thr Phe Ala Val Gly Val Asn Ile Cys Leu Gly
1 5 10
Phe Thr Ala
        15
Azurocidin signal Sequence
SEQ ID NO: 7
Met Thr Arg Leu Thr Val Leu Ala Leu Leu Ala Gly Leu Leu Ala Ser
Ser Arg Ala
ENPP7 signal sequence
SEQ. ID NO: 16
Met Arg Gly Pro Ala Val Leu Leu Thr Val Ala Leu Ala Thr Leu Leu
1 5 10 15
Ala Pro Gly Ala
                        20
ENPP7 signal sequence
SEQ. ID NO: 17
Met Arg Gly Pro Ala Val Leu Leu Thr Val Ala Leu Ala Thr Leu Leu
1 5 10 15
Ala Pro Gly Ala Gly Ala
                        20
ENPP5 signal sequence
SEQ. ID NO: 18
Met Thr Ser Lys Phe Leu Leu Val Ser Phe Ile Leu Ala Ala Leu Ser
1 5 10 15
Leu Ser Thr Thr Phe Ser
                        20
ENPP121GLK signal sequence
SEQ ID NO: 19
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala Gly
                                85 90 95
Leu Lys
ENPP121 signal sequence
SEQ ID NO: 20
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala
                                85 90 95
```

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, different elements of the fusion proteins (e.g., immunoglobulin Fc fusion proteins) may be arranged in any manner that is consistent with desired functionality. For example, a soluble ENPP1 polypeptide domain may be placed C-terminal to a heterologous protein portion, or alternatively, a heterologous protein portion may be placed C-terminal to a soluble ENPP1 polypeptide domain. The soluble ENPP1 polypeptide domain and the heterologous protein portion may be directly or indirectly linked in a fusion protein, and additional domains or amino acid sequences may be included C- or N-terminal to either domain or between the domains. Preferred fusion proteins comprise the amino acid sequence set forth in any one of SEQ ID NOs: 9-11. In some embodiments, the ENPP1 fusion polypeptide consists of or comprises SEQ ID NO:9. In some embodiments, the ENPP1 fusion polypeptide consists of or comprises SEQ ID NO:10. In some embodiments, the ENPP1 fusion polypeptide consists of or comprises SEQ ID NO:11.

In some embodiments, soluble ENPP1 polypeptides of the present disclosure contain one or more heterologous moieties. Optionally, a soluble ENPP1 polypeptide includes one or more heterologous moieties selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, a soluble ENPP1 polypeptide disclosed herein is further modified. Such modifications include, but are not limited to, acetylation, carboxylation, glycosylation, phosphorylation, lipidation, and acylation. As a result, the soluble ENPP1 polypeptide may contain non-amino acid elements, such as polyethylene glycols, lipids, polysaccharide or monosaccharide, and phosphates. Effects of such non-amino acid elements on the functionality of a soluble ENPP1 polypeptide may be tested as described herein for other soluble ENPP1 polypeptides. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the soluble ENPP1 polypeptides.

As used herein, percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. In some embodiments, alignment is performed using the CLUSTAL OMEGA software. Those skilled in the art can determine appropriate parameters for aligning sequences, including any algorithms needed to achieve maximal alignment over the full length of the sequences being compared.

In some embodiments, the activity of soluble ENPP1 polypeptides may also be tested in a cell-based or in vivo assay. For example, the effect of a soluble ENPP1 polypeptide on the production of inorganic pyrophosphates (PPi) can be measured. Specifically, the pyrophosphatase/phosphodiesterase domain of an ENPP1 protein hydrolyzes extracellular nucleotide triphosphates to produce inorganic pyrophsphates (PPi) and is generally soluble. This activity can be measured using a pNP-TMP assay as well as an HPLC-based ATP hydrolysis assay, as previously described (Saunders, et al., 2008, Mol. Cancer Ther. 7 (10): 3352-62; Albright, et al., 2015, Nat Comm. 6:10006). The effect of soluble ENPP1 polypeptides on the expression of genes involved in ENPP1 associated diseases such as ARHR2 (e.g., transcription of fibroblast growth factor 23 in osteoblasts and osteoclasts) can be assessed. This may, as needed, be performed in the presence of one or more nucleotide triphosphates or other ENPP1 substrates, and cells may be transfected so as to produce a soluble ENPP1 polypeptide. Likewise, a soluble ENPP1 polypeptide may be administered to a mouse or other animal and effects on ENPP1 associated diseases may be assessed using art-recognized methods.

In some embodiments, ENPP1 polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) analyses. Methods for assessment of purity are well known in the art [see, e.g., Flatman et al., (2007) J. Chromatogr. B 848:79-87]. In some embodiments, soluble ENPP1 polypeptides to be used in accordance with the methods described herein are recombinant polypeptides.

ENPP1 polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus, Yeast Pichia] as is well known in the art. The protein can be produced in either adherent or suspension cells. In some embodiments, the fusion protein is expressed in CHO cells. To establish stable cell lines the nucleic acid sequence encoding ENPP1 constructs are cloned into an appropriate vector for large scale protein production. In a further embodiment, the modified or unmodified polypeptides of the disclosure may be produced by digestion of recombinantly produced full-length ENPP1 polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ENPP1 polypeptides using chemical cleavage (e.g., cyanogen bromide, hydroxylamine, etc.).

Many expression systems are known and can be used for the production of ENPP1 fusion protein, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired protein can be produced in conventional ways, for example from a coding sequence inserted in the host chromosome or on a free plasmid.

The yeasts can be transformed with a coding sequence for the desired protein in any of the usual ways (e.g., electroporation). Methods for transformation of yeast by electroporation are disclosed in Becker & Guarente, 1990, Methods Enzymol. 194:182. Successfully transformed cells, i.e., cells that contain a DNA construct of the present disclosure, can be identified by well-known techniques. For example, cells resulting from the introduction of an expression construct can be grown to produce an ENPP1 polypeptide. Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available front Stratagene Cloning Systems, La Jolla, CA, USA Plasmids pRS403, pRS404, pRS405 and pRS406 are Yeast Integrating plasmids (Yips) and incorporate the yeast selectable markers I-11S3, TRP1, LEU2 and 1JRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. As a result, the products of the reaction are DNA segments carrying polymeric linker sequences at their ends. These DNA segments can be cleaved with an appropriate restriction enzyme and ligated to an expression vector that has been cleaved with an enzyme that produces termini compatible with those of the DNA segment.

Clones of single, stably transfected cells are then established and screened for high expressing clones of the desired ENPP1 fusion protein. Screening of the single cell clones for ENPP1 protein expression can be accomplished in a high-throughput manner in 96 well plates using the synthetic enzymatic substrate pNP-TMP as previously described (Albright, et al., 2015, Nat. Commun. 6:10006). Upon identification of high expressing clones through screening, protein production can be accomplished in shaking flasks or bio-reactors are previously described in Albright, et al., 2015, Nat. Commun. 6:10006.

Purification of ENPP1 can be accomplished using a combination of standard purification techniques known in the art. Following purification, ENPP1-Fc can be dialyzed into PBS supplemented with Zn²⁺ and Mg²⁺ (PBSplus) concentrated to between 5 and 7 mg/ml, and frozen at −80° C. in aliquots of 200-500 pl. Aliquots can be thawed immediately prior to use and the specific activity of the solution can be adjusted to 31.25 au/ml (or about 0.7 mg/ml depending on the preparation) by dilution in PBSplus.

### 4. Exemplary Uses

In certain aspects, the present disclosure relates to the use of certain soluble ENPP1 polypeptides (e.g., and fusion proteins thereof) for reducing and/or preventing progression of pathological calcification in a subject in need thereof, the method comprising administering to the subject a therapeutically effective amount of the polypeptide fusion and/or the soluble polypeptide disclosed herein (e.g., SEQ ID NOs: 2, 9, 10, and 11). In certain embodiments, the pathological calcification is selected from the group consisting of idiopathic infantile arterial calcification (IIAC) and calcification of atherosclerotic plaques. In certain embodiments, the pathological ossification is selected from the group consisting of ossification of the posterior longitudinal ligament (OPLL), hypophosphatemic rickets, and osteoarthritis.

In some embodiments, the disclosure contemplates methods of reducing or preventing progression of pathological ossification in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of reducing or preventing progression of ectopic calcification of soft tissue, including reducing, ameliorating, or preventing vascular calcification, in a subject in need thereof, the method comprising administering to the subject a therapeutically effective amount of a soluble ENPP1 polypeptide or ENPP1 fusion polypeptide disclosed herein.

In some embodiments, the disclosure contemplates methods of reducing or preventing progression of diseases caused by an ENPP1 deficiency (e.g., GACI and ARHR2). ENPP1 deficiency is characterized by reduced levels of ENPP1 activity and or defective expression of ENPP1 levels (compared to that of ENPP1 activity levels or ENPP1 expression levels respectively in normal healthy subjects) in a subject in need thereof, the method comprising administering to the subject a therapeutically effective amount of a soluble ENPP1 polypeptide or ENPP1 fusion protein disclosed herein (e.g., SEQ ID NOs: 2, 9, 10, and 11). In some embodiments, the ENPP1 deficiency is GACI. In some embodiments, the ENPP1 deficiency is ARHR2.

In some embodiments, the disclosure contemplates methods of reducing or preventing progression of diseases caused by lower levels of plasma PPi in a subject in need thereof, the method comprising administering to the subject a therapeutically effective amount of the polypeptides disclosed herein to increase the plasma PPi of the subjects to normal or above (30-50% higher than) normal levels and then to maintain the plasma PPi at a constant normal or above normal level thereafter. A normal level of Plasma ppi corresponds to 2-5 μM, in some embodiments the normal level is 2-3 μM. (Bernhard et al., A Reference range for Plasma levels of Inorganic Pyrophosphate in Children using the ATP Sulfurylase method, Journal of Clinical Endocrinology & Metabolism, 2021) The method further comprises administering additional therapeutic effective amounts at intervals of two days, three days, one week or one month in order to maintain the Plasma PPi of the subject at a constant normal or above normal level to reduce or prevent the progression of pathological calcification or ossification. In certain embodiments, a soluble ENPP1 polypeptide or ENPP1 fusion polypeptide disclosed herein can be used to raise pyrophosphate (PPi) levels in a subject having PPi level lower than normal level. In other embodiments, a soluble ENPP1 polypeptide or ENPP1 fusion polypeptide disclosed herein can be used to reduce or prevent progression of pathological calcification or ossification in a subject having PPi levels lower than normal level. In some embodiments, a soluble ENPP1 polypeptide or ENPP1 fusion polypeptide disclosed herein can be used to treat ENPP1 deficiency (e.g., GACI and ARHR2) manifested by a reduction of extracellular PPi concentration in a subject. In certain embodiments, the steady state level of plasma PPi achieved after administration of a first dosage of a soluble ENPP1 polypeptide or ENPP1 fusion polypeptide disclosed herein is maintained for a time period of at least 2 days, at least 4 days, at least a week or at least a month.

In some embodiments, the disclosure contemplates methods of reducing or preventing progression of a disease caused by lower than normal levels of plasma PPi in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11) to increase and/or sustain the plasma PPi of the subjects to a level that is about 90%, 95%, 100%, 105%, 110%, 120%, 130%, 140%, or 150% of the normal PPi level. In certain embodiments, the method further comprises further administration of the polypeptide disclosed herein every two days, three days, one week, or one month in order to maintain the plasma PPi levels at a level that is about 90%, 95%, 100%, 105%, 110%, 120%, 130%, 140%, or 150% of the normal PPi level, thus preventing the progression of pathological calcification or ossification.

In certain embodiments, a second dosage of a soluble ENPP1 polypeptide or ENPP1 fusion polypeptide disclosed herein is administered after a suitable time interval of about after two days, after four days, after a week, or after a month to the subject so that the steady state level of plasma PPi is maintained at a constant or steady state level and does not return to the lower level of PPi that the subject had prior to the administration of first dosage of constructs disclosed herein.

Without wishing to be bound be theory, it is believed that maintaining a steady state concentration of plasma PPi at normal levels reduces and/or prevents progression of pathological calcification and pathological ossification of subjects.

In some embodiments, the disclosure contemplates methods of treating, reversing, or preventing progression of ossification of the posterior longitudinal ligament (OPLL) in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reverting, or preventing progression of hypophosphatemic rickets in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, a soluble ENPP1 polypeptide of the disclosure treats human or animal disorders or conditions such as ectopic calcification (e.g., soft tissue calcification, arterial calcification, and vascular calcification), chronic kidney disease (CKD), end stage renal disease (ESRD), calcific uremic arteriolopathy (CUA), calciphylaxis, ossification of the posterior longitudinal ligament (OPLL), hypophosphatemic rickets, osteoarthritis, aging related hardening of arteries, idiopathic infantile arterial calcification (IIAC), calcification of atherosclerotic plaques, ENPP1 deficiencies [e.g., autosomal recessive hypophosphatemic rickets type 2 (ARHR2) and Generalized Arterial Calcification of Infancy (GACI)], disorders associated with a pathogenic mutation in ABCC6 gene [(e.g., ABCC6 Deficiency), such as pseudoxanthoma elasticum (PXE)], and pathological ossification. Examples of ENPP1 polypeptides include human ENPP1 precursor polypeptide (e.g., SEQ ID NO: 1), and soluble human ENPP1 polypeptides (e.g., SEQ ID NOs: 2, 9, 10, and 11).

In other embodiments, the soft tissue comprises atherosclerotic plaques. In some embodiments, the soft tissue comprises muscular arteries. In some embodiments, the soft tissue is selected from the group consisting of joint and spine. In some embodiments, the joint is selected from the group consisting of joints of the hands and joints of the feet. In some embodiments, the soft tissue is selected from the group consisting of articular cartilage and vertebral disk cartilage. In some embodiments, the soft tissue comprises vessels. In some embodiments, the soft tissue comprises connective tissue.

In some embodiments, the disclosure contemplates methods of treating, reversing, or preventing progression of Pseudoxanthoma Elasticum (PXE) in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of reducing or preventing progression of age-related hardening of arteries in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of calcification of atherosclerotic plaques in vascular arteries in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of osteoarthritis in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of hardening of arteries due to progeria in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of X-linked hypophosphatemic rickets (XLH), hereditary hypophosphatemic rickets (HHRH), hypophosphatemic bone disease (HBD), Ossification of Posterior Longitudinal Ligament (OPLL), autosomal dominant hypophosphatemic rickets (ADHR), and/or and autosomal recessive hypophosphatemic rickets (ARHR2) in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of age-related osteopenia in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of ankylosing spondylitis in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In some embodiments, the disclosure contemplates methods of treating, reversing, or preventing progression of strokes in pediatric sickle cell anemia in a subject in need thereof, the method comprising administering to the subject a therapeutically effective amount of a soluble ENPP1 polypeptide or ENPP1 fusion polypeptide disclosed herein (e.g., SEQ ID NOs: 2, 9, 10, and 11).

In certain embodiments, the disclosure contemplates methods of treating, reversing, or preventing progression of disease in a subject diagnosed with progeria, the method comprising administering to the subject a therapeutically effective amount of a soluble ENPP1 polypeptide or ENPP1 fusion polypeptide disclosed herein (e.g., SEQ ID NOs: 2, 9, 10, and 11).

In certain embodiments, the polypeptide is a secreted product of a ENPP1 precursor protein expressed in a mammalian cell. In other embodiments, the ENPP1 precursor protein comprises a signal peptide sequence and an ENPP1 polypeptide, wherein the ENPP1 precursor protein undergoes proteolytic processing to the polypeptide disclosed herein. In some embodiments, in the ENPP1 precursor protein the signal peptide sequence is conjugated to the ENPP1 polypeptide N-terminus. Upon proteolysis, the signal sequence is cleaved from the ENPP1 precursor protein to provide the ENPP1 polypeptide. In certain embodiments, the signal peptide sequence is selected from the group consisting of ENPP1 signal peptide sequence, ENPP2 signal peptide sequence, ENPP7 signal peptide sequence, and ENPP5 signal peptide sequence.

In certain embodiments, the polypeptide is administered acutely or chronically to the subject. In other embodiments, the polypeptide is administered locally, regionally, parenterally or systemically to the subject.

In certain embodiments, the subject is a mammal. In other embodiments, the mammal is human.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present ENPP1 polypeptides may provide benefit.

In certain aspects, the disclosure relates to methods for preventing diseases and disorders in a subject, in that a soluble ENPP1 polypeptide or ENPP1 fusion polypeptide disclosed herein can be administered to a subject prior to the onset of the disease or disorder, thereby preventing the disease or disorder from developing. Therefore, the disclosure relates to methods for preventing or delaying onset, or reducing progression or growth, of a disease or disorder in a subject, comprising administering an ENPP1 polypeptide to a subject prior to detection of the disease or disorder. In certain embodiments, the ENPP1 polypeptide is administered to a subject with a strong family history of the disease or disorder, thereby preventing or delaying onset or progression of the disease or disorder.

Armed with the disclosure herein, one skilled in the art would thus appreciate that the prevention of a disease or disorder in a subject encompasses administering to a subject an ENPP1 polypeptide as a preventative measure against the disease or disorder.

In certain embodiments, lyophilized formulations disclosed herein may be administered to deliver a dose of between 1 ng/kg/day and 100 mg/kg/day. In other embodiments, the lyophilized formulations disclosed herein may be administered to deliver a dose of between 1 ng/kg/day and 500 mg/kg/day.

In some embodiments, lyophilized formulations disclosed herein may be administered to deliver a dose of 0.1 mg/kg. In some embodiments, lyophilized formulations disclosed herein may be administered to deliver a dose of 0.2 mg/kg. In some embodiments, lyophilized formulations disclosed herein may be administered to deliver a dose of 0.3 mg/kg. In some embodiments, lyophilized formulations disclosed herein may be administered to deliver a dose of 0.4 mg/kg. In some embodiments, lyophilized formulations disclosed herein may be administered to deliver a dose of 0.5 mg/kg. In other embodiments, lyophilized formulations disclosed herein may be administered to deliver a dose of 0.6 mg/kg. In other embodiments, lyophilized formulations disclosed herein may be administered to deliver a dose of 0.7 mg/kg. In other embodiments, lyophilized formulations disclosed herein may be administered to deliver a dose of 0.8 mg/kg. In other embodiments, lyophilized formulations disclosed herein may be administered to deliver a dose of 0.9 mg/kg. In other embodiments, lyophilized formulations disclosed herein may be administered to deliver a dose of 1 mg/kg. In other embodiments, lyophilized formulations disclosed herein may be administered to deliver a dose of 1.2 mg/kg. In other embodiments, lyophilized formulations disclosed herein may be administered to deliver a dose of 1.4 mg/kg. In other embodiments, lyophilized formulations disclosed herein may be administered to deliver a dose of 1.6 mg/kg. In other embodiments, lyophilized formulations disclosed herein may be administered to deliver a dose of 1.8 mg/kg.

The relative amounts of the active ingredient (e.g., soluble ENPP1 polypeptides and fusion proteins thereof), the pharmaceutically acceptable carrier, and any additional ingredients in a lyophilized formulation disclosed herein will vary, depending upon the identity, size, and condition of the subject treated and further depending upon the route by which the composition is to be administered. By way of example, the composition may comprise between about 0.1% and about 100% (w/w) active ingredient.

As used herein, a “unit dose” is a discrete amount of the lyophilized formulation comprising a predetermined amount of the active ingredient (e.g., soluble ENPP1 polypeptides and fusion proteins thereof). The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In some embodiments, ENPP1-Fc may be present in the formulation at a concentration ranging from about 0.1 to about 300 mg/ml. In some embodiments the concentration of ENPP1-Fc is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 70 mg/ml, about 80 mg/ml, about 90 mg/ml, about 100 mg/ml, about 101 mg/ml, about 102 mg/ml, about 102.5 mg/ml, about 103 mg/ml, about 103.5 mg/ml, about 104 mg/ml, about 104.5 mg/ml, about 105 mg/ml, about 105.5 mg/ml, about 106 mg/ml, about 106.5 mg/ml, about 107 mg/ml, about 107.5 mg/ml, about 108 mg/ml, about 108.5 mg/ml, about 109 mg/ml, about 109.5 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, about 150 mg/ml, about 151 mg/ml, about 152 mg/ml, about 153 mg/ml, about 154 mg/ml, about 155 mg/ml, about 156 mg/ml, about 157 mg/ml, about 158 mg/ml, about 159 mg/ml, about 160 mg/ml, about 170 mg/ml, about 180 mg/ml, about 190 mg/ml, about 200 mg/ml, about 201 mg/ml, about 202 mg/ml, about 203 mg/ml, about 204 mg/ml, about 205 mg/ml, about 206 mg/ml, about 207 mg/ml, about 208 mg/ml, about 209 mg/ml, about 210 mg/ml, about 211 mg/ml, about 212 mg/ml, about 213 mg/ml, about 214 mg/ml, about 215 mg/ml, about 216 mg/ml, about 217 mg/ml, about 218 mg/ml, about 219 mg/ml, about 220 mg/ml, about 221 mg/ml, about 222 mg/ml, about 223 mg/ml, about 224 mg/ml, about 225 mg/ml, about 226 mg/ml, about 227 mg/ml, about 228 mg/ml, about 229 mg/ml, about 230 mg/ml, about 231 mg/ml, about 232 mg/ml, about 233 mg/ml, about 234 mg/ml, about 235 mg/ml, about 236 mg/ml, about 237 mg/ml, about 238 mg/ml, about 239 mg/ml, about 240 mg/ml, about 241 mg/ml, about 242 mg/ml, about 243 mg/ml, about 244 mg/ml, about 245 mg/ml, about 246 mg/ml, about 247 mg/ml, about 248 mg/ml, about 249 mg/ml, about 250 mg/ml, about 251 mg/ml, about 252 mg/ml, about 253 mg/ml, about 254 mg/ml, about 255 mg/ml, about 256 mg/ml, about 257 mg/ml, about 258 mg/ml, about 259 mg/ml, about 260 mg/ml, about 270 mg/ml, about 280 mg/ml, about 290 mg/ml, or about 300 mg/ml.

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure (e.g., soluble ENPP1 polypeptides and fusion proteins thereof) to a patient, such as a mammal (i.e., a human), may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure is administered at an appropriate time interval of every 2 days, or every 4 days, or every week or every month so as to achieve a continuous level of plasma PPi that is either close to the normal (1-3 pM) level or above (30-50% higher than) normal levels of PPi. Therapeutic dosage of the ENPP1 polypeptides may also be determined based on half-life or the rate at which the therapeutic polypeptide is cleared out of the body. The polypeptide according to the disclosure is administered at appropriate time intervals of either every 2 days, or every 4 days, every week or every month so as to achieve a constant level of enzymatic activity of ENPP1.

For example, several divided doses may be administered daily or the dose may be proportionally reduced as indicated by the exigencies of the therapeutic situation. A nonlimiting example of an effective dose range for a therapeutic compound disclosed herein is from about 0.01 and 50 mg/kg of body weight/per day. In some embodiments, the effective dose range for a therapeutic compound disclosed herein is from about 50 ng to 500 ng/kg, preferably 100 ng to 300 ng/kg of body weight. One of ordinary skill in the art would be able to study the relevant factors and make the determination regarding the effective amount of the therapeutic compound without undue experimentation.

The soluble ENPP1 polypeptides and fusion proteins thereof may be administered to an patient as frequently as several times daily, or it may be administered less frequently, such as once a day, once a week, once every two weeks, once a month, or even less frequently, such as once every several months or even once a year or less. It is understood that the amount of soluble ENPP1 polypeptides and fusion proteins thereof dosed per day may be administered, in nonlimiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient.

Actual dosage levels of the active ingredients (e.g., soluble ENPP1 polypeptides and fusion proteins thereof) in the lyophilized formulations of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the lyophilized formulation required. For example, the physician or veterinarian could start doses of the compounds disclosed herein employed in the lyophilized formulation at levels lower than that required in order to achieve the desired therapeutic effect and gradually increase the dosage until the desired effect is achieved.

In certain embodiments, the compositions disclosed herein are administered to the patient in dosages that range from one to five times per day or more. In other embodiments, the compositions disclosed herein are administered to the patient in range of dosages that include, but are not limited to, once every day, every two, days, every three days to once a week, and once every two weeks. The frequency of administration of the various combination compositions disclosed herein varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged lyophilized formulation comprising a container holding a therapeutically effective amount of a compound disclosed herein, alone or in combination with a second pharmaceutical agent; and instructions for using the compound to treat, prevent, or reduce one or more symptoms of a disease or disorder in a patient.

In certain embodiments, the polypeptide, or its precursor protein, is administered acutely or chronically to the subject. In other embodiments, the polypeptide, or its precursor protein, is administered locally, regionally or systemically to the subject. In yet another embodiment, the polypeptide, or its precursor protein, is delivered on an encoded vector, wherein the vector encodes the protein and it is transcribed and translated from the vector upon administration of the vector to the subject.

As used herein, “parenteral administration” of a lyophilized formulation includes any route of administration characterized by physical breaching of a tissue of a subject and administration of the lyophilized formulation through the breach in the tissue. Parenteral administration thus includes, but is not limited to, administration of a lyophilized formulation by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrastemal injection, and kidney dialytic infusion techniques.

Lyophilized formulations suitable for parenteral administration may comprise one or more ENPP1 polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the lyophilized formulation of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### 5. Sequences

TABLE 2
Sequences

| SEQ ID NO | Sequence | Description |
|---|---|---|
| 1 | 1 MERDGCAGGG SRGGEGGRAP REGPAGNGRD RGRSHAAEAP GDPQAAASLL | Full, unprocessed |
| | 51 APMDVGEEPL EKAARARTAK DPNTYKVLSL VLSVCVLTTI LGCIFGLKPS | amino acid |
| | 101 CAKEVKSCKG RCFERTFGNC RCDAACVELG NCCLDYQETC IEPEHIWTCN | sequence of wild- |
| | 151 KFRCGEKRLT RSLCACSDDC KDKGDCCINY SSVCQGEKSW VEEPCESINE | type ENPP1 |
| | 201 PQCPAGFETP PTLLFSLDGF RAEYLHTWGG LLPVISKLKK CGTYTKNMRP | precursor protein |
| | 251 VYPTKTFPNH YSIVTGLYPE SHGIIDNKMY DPKMNASFSL KSKEKFNPEW | |
| | 301 YKGEPIWVTA KYQGLKSGTF FWPGSDVEIN GIFPDIYKMY NGSVPFEERI | |
| | 351 LAVLQWLQLP KDERPHFYTL YLEEPDSSGH SYGPVSSEVI KALQRVDGMV | |
| | 401 GMLMDGLKEL NLHRCLNLIL ISDHGMEQGS CKKYIYLNKY LGDVKNIKVI | |
| | 451 YGPAARLRPS DVPDKYYSFN YEGIARNLSC REPNQHFKPY LKHFLPKRLH | |
| | 501 FAKSDRIEPL TFYLDPQWQL ALNPSERKYC GSGFHGSDNV FSNMQALFVG | |
| | 551 YGPGFKHGIE ADTFENIEVY NLMCDLLNLT PAPNNGTHGS LNHLLKNPVY | |
| | 601 TPKHPKEVHP LVQCPFTRNP RDNLGCSCNP SILPIEDFQT QFNLTVAEEK | |
| | 651 IIKHETLPYG RPRVLQKENT ICLLSQHQFM SGYSQDILMP LWTSYTVDRN | |
| | 701 DSFSTEDFSN CLYQDFRIPL SPVHKCSFYK NNTKVSYGFL SPPQLNKNSS | |
| | 751 GIYSEALLTT NIVPMYQSFQ VIWRYFHDTL LRKYAEERNG VNVVSGPVED | |
| | 801 FDYDGRCDSL ENLRQKRRVI RNQEILIPTH FFIVLTSCKD TSQTPLHCEN | |
| | 851 LDTLAFILPH RTDNSESCVH GKHDSSWVEE LLMLHRARIT DVEHITGLSF | |
| | 901 YQQRKEPVSD ILKLKTHLPT FSQED | |
| 2 | 1 PSCAKEVKSC KGRCFERTFG NCRCDAACVE LGNCCLDYQE TCIEPEHIWT | The processed |
| | 51 CNKFRCGEKR LTRSLCACSD DCKDKGDCCI NYSSVCQGEK SWVEEPCESI | (mature) |
| | 101 NEPQCPAGFE TPPTLLFSLD GFRAEYLHTW GGLLPVISKL KKCGTYTKNM | extracellular |
| | 151 RPVYPTKTFP NHYSIVTGLY PESHGIIDNK MYDPKMNASF SLKSKEKFNP | ENPP1 |
| | 201 EWYKGEPIWV TAKYQGLKSG TFFWPGSDVE INGIFPDIYK MYNGSVPFEE | polypeptide |
| | 251 RILAVLQWLQ LPKDERPHFY TLYLEEPDSS GHSYGPVSSE VIKALQRVDG | sequence |
| | 301 MVGMLMDGLK ELNLHRCLNL ILISDHGMEQ GSCKKYIYLN KYLGDVKNIK | |
| | 351 VIYGPAARLR PSDVPDKYYS FNYEGIARNL SCREPNQHFK PYLKHFLPKR | |
| | 401 LHFAKSDRIE PLTFYLDPQW QLALNPSERK YCGSGFHGSD NVFSNMQALF | |
| | 451 VGYGPGFKHG IEADTFENIE VYNLMCDLLN LTPAPNNGTH GSLNHLLKNP | |
| | 501 VYTPKHPKEV HPLVQCPFTR NPRDNLGCSC NPSILPIEDE QTQFNLTVAE | |
| | 551 EKIIKHETLP YGRPRVLQKE NTICLLSQHQ FMSGYSQDIL MPLWTSYTVD | |
| | 601 RNDSFSTEDF SNCLYQDFRI PLSPVHKCSF YKNNTKVSYG FLSPPQLNKN | |
| | 651 SSGIYSEALL TTNIVPMYQS FQVIWRYFHD TLLRKYAEER NGVNVVSGPV | |
| | 701 FDFDYDGRCD SLENLRQKRR VIRNQEILIP THFFIVLTSC KDTSQTPLHC | |
| | 751 ENLDTLAFIL PHRTDNSESC VHGKHDSSWV EELLMLHRAR ITDVEHITGL | |
| | 801 SFYQQRKEPV SDILKLKTHL PTFSQED | |
| 9 | FTAGLKPSCAKEVKSCKGRCFERTFGNCRCDAACVELGNCCLDYQETCIEPEHIWTCN | ENPP1-linker- |
| | KFRCGEKRLTRSLCACSDDCKDKGDCCINYSSVCQGEKSWVEEPCESINEPQCPAGFE | hIgG1 Fc |
| | TPPTLLFSLDGFRAEYLHTWGGLLPVISKLKKCGTYTKNMRPVYPTKTFPNHYSIVTG | construct |
| | LYPESHGIIDNKMYDPKMNASFSLKSKEKFNPEWYKGEPIWVTAKYQGLKSGTFFWPG | |

| | | |
|---|---|---|
| | SDVEINGIFPDIYKMYNGSVPFEERILAVLQWLQLPKDERPHFYTLYLEEPDSSGHSY | |
| | GPVSSEVIKALQRVDGMVGMLMDGLKELNLHRCLNLILISDHGMEQGSCKKYIYLNKY | |
| | LGDVKNIKVIYGPAARLRPSDVPDKYYSFNYEGIARNLSCREPNQHFKPYLKHFLPKR | |
| | LHFAKSDRIEPLTFYLDPQWQLALNPSERKYCGSGFHGSDNVFSNMQALFVGYGPGFK | |
| | HGIEADTFENIEVYNLMCDLLNLTPAPNNGTHGSLNHLLKNPVYTPKHPKEVHPLVQC | |
| | PFTRNPRDNLGCSCNPSILPIEDFQTQFNLTVAEEKIIKHETLPYGRPRVLQKENTIC | |
| | LLSQHQFMSGYSQDILMPLWTSYTVDRNDSFSTEDFSNCLYQDFRIPLSPVHKCSFYK | |
| | NNTKVSYGFLSPPQLNKNSSGIYSEALLTTNIVPMYQSFQVIWRYFHDTLLRKYAEER | |
| | NGVNVVSGPVFDFDYDGRCDSLENLRQKRRVIRNQEILIPTHFFIVLTSCKDTSQTPL | |
| | HCENLDTLAFILPHRTDNSESCVHGKHDSSWVEELLMLHRARITDVEHITGLSFYQQR | |
| | KEPVSDILKLKTHLPTFSQEDLINDKTHTCPPCPAPELLGGPSVFLFPPKPKDTLMIS | |
| | RTPEVTCVVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSREEMTKNQVSLTCLVK | |
| | GFYPSDIAVEWESNGQPENNYKTTPPVLDSDGSFFLYSKLTVDKSRWQQGNVFSCSVM | |
| | HEALHNHYTQKSLSLSPGK | |
| 10 | GLKPSCAKEVKSCKGRCFERTFGNCRCDAACVELGNCCLDYQETCIEPEHIWTCNKFR | ENPP1-linker- |
| | CGEKRLTRSLCACSDDCKDKGDCCINYSSVCQGEKSWVEEPCESINEPQCPAGFETPP | hIgG1 Fc |
| | TLLFSLDGFRAEYLHTWGGLLPVISKLKKCGTYTKNMRPVYPTKTFPNHYSIVTGLYP | construct |
| | ESHGIIDNKMYDPKMNASFSLKSKEKFNPEWYKGEPIWVTAKYQGLKSGTFFWPGSDV | |
| | EINGIFPDIYKMYNGSVPFEERILAVLQWLQLPKDERPHFYTLYLEEPDSSGHSYGPV | |
| | SSEVIKALQRVDGMVGMLMDGLKELNLHRCLNLILISDHGMEQGSCKKYIYLNKYLGD | |
| | VKNIKVIYGPAARLRPSDVPDKYYSFNYEGIARNLSCREPNQHFKPYLKHFLPKRLHF | |
| | AKSDRIEPLTFYLDPQWQLALNPSERKYCGSGFHGSDNVFSNMQALFVGYGPGFKHGI | |
| | EADTFENIEVYNLMCDLLNLTPAPNNGTHGSLNHLLKNPVYTPKHPKEVHPLVQCPFT | |
| | RNPRDNLGCSCNPSILPIEDFQTQFNLTVAEEKIIKHETLPYGRPRVLQKENTICLLS | |
| | QHQFMSGYSQDILMPLWTSYTVDRNDSFSTEDFSNCLYQDFRIPLSPVHKCSFYKNNT | |
| | KVSYGFLSPPQLNKNSSGIYSEALLTTNIVPMYQSFQVIWRYFHDTLLRKYAEERNGV | |
| | NVVSGPVFDFDYDGRCDSLENLRQKRRVIRNQEILIPTHFFIVLTSCKDTSQTPLHCE | |
| | NLDTLAFILPHRTDNSESCVHGKHDSSWVEELLMLHRARITDVEHITGLSFYQQRKEP | |
| | VSDILKLKTHLPTFSQEDLINDKTHTCPPCPAPELLGGPSVFLFPPKPKDTIMISRTP | |
| | EVTCVVVDVSHEDPEVKFNWYVDGVEVHNAKTKPREEQYNSTYRVVSVLTVLHQDWLN | |
| | GKEYKCKVSNKALPAPIEKTISKAKGQPREPQVYTLPPSREEMTKNQVSLTCLVKGFY | |
| | PSDIAVEWESNGQPENNYKTTPPVLDSDGSFFLYSKLTVDKSRWQQGNVFSCSVMHEA | |
| | LHNHYTQKSLSLSPGK | |
| 11 | PSCAKEVKSCKGRCFERTFGNCRCDAACVELGNCCLDYQETCIEPEHIWTCNKFRCGE | ENPP1-linker- |
| | KRLTRSLCACSDDCKDKGDCCINYSSVCQGEKSWVEEPCESINEPQCPAGFETPPTLL | hIgG1 Fc |
| | FSLDGFRAEYLHTWGGLLPVISKLKKCGTYTKNMRPVYPTKTFPNHYSIVTGLYPESH | construct |
| | GIIDNKMYDPKMNASFSLKSKEKFNPEWYKGEPIWVTAKYQGLKSGTFFWPGSDVEIN | |
| | GIFPDIYKMYNGSVPFEERILAVLQWLQLPKDERPHFYTLYLEEPDSSGHSYGPVSSE | |
| | VIKALQRVDGMVGMLMDGLKELNLHRCLNLILISDHGMEQGSCKKYIYLNKYLGDVKN | |
| | IKVIYGPAARLRPSDVPDKYYSFNYEGIARNLSCREPNQHFKPYLKHFLPKRLHFAKS | |
| | DRIEPLTFYLDPQWQLALNPSERKYCGSGFHGSDNVFSNMQALFVGYGPGFKHGIEAD | |
| | TFENIEVYNLMCDLLNLTPAPNNGTHGSLNHLLKNPVYTPKHPKEVHPLVQCPFTRNP | |
| | RDNLGCSCNPSILPIEDFQTQFNLTVAEEKIIKHETLPYGRPRVLQKENTICLLSQHQ | |
| | FMSGYSQDILMPLWTSYTVDRNDSFSTEDFSNCLYQDFRIPLSPVHKCSFYKNNTKVS | |
| | YGFLSPPQLNKNSSGIYSEALLTTNIVPMYQSFQVIWRYFHDTLLRKYAEERNGVNVV | |
| | SGPVFDFDYDGRCDSLENLRQKRRVIRNQEILIPTHFFIVLTSCKDTSQTPLHCENLD | |
| | TLAFILPHRTDNSESCVHGKHDSSWVEELLMLHRARITDVEHITGLSFYQQRKEPVSD | |
| | ILKLKTHLPTFSQEDLINDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQDWINGKE | |
| | YKCKVSNKALPAPIEKTISKAKGQPREPQVYTLPPSREEMTKNQVSLTCLVKGFYPSD | |
| | IAVEWESNGQPENNYKTTPPVLDSDGSFFLYSKLTVDKSRWQQGNVESCSVMHEALHN | |
| | HYTQKSLSLSPGK | |
| 12 | DKTHTCPPCPAPELLGGPSVFLFPPKPKDTLMISRTPEVTCVVVDVSHEDPEVKENWY | Human IgG1 Fc |
| | VDGVEVHNAKTKPREEQYNSTYRVVSVLTVLHQDWLNGKEYKCKVSNKALPAPIEKTI | |
| | SKAKGQPREPQVYTLPPSREEMTKNQVSLTCLVKGFYPSDIAVEWESNGQPENNYKTT | |
| | PPVLDSDGSFFLYSKLTVDKSRWQQGNVFSCSVMHEALHNHYTQKSLSLSPGK | |
| 13 | FTAGLKPSCAKEVKSCKGRCFERTFGNCRCDAACVELGNCCLDYQETCIEPEHIWTCN | Exemplary |
| | KFRCGEKRLTRSLCACSDDCKDKGDCCINYSSVCQGEKSWVEEPCESINEPQCPAGFE | soluble form of |
| | TPPTLLFSLDGFRAEYLHTWGGLLPVISKLKKCGTYTKNMRPVYPTKTFPNHYSIVTG | ENPP1 |
| | LYPESHGIIDNKMYDPKMNASFSLKSKEKFNPEWYKGEPIWVTAKYQGLKSGTFFWPG | |
| | SDVEINGIFPDIYKMYNGSVPFEERILAVLQWLQLPKDERPHFYTLYLEEPDSSGHSY | |
| | GPVSSEVIKALQRVDGMVGMLMDGLKELNLHRCLNLILISDHGMEQGSCKKYIYLNKY | |
| | LGDVKNIKVIYGPAARLRPSDVPDKYYSFNYEGIARNLSCREPNQHFKPYLKHFLPKR | |
| | LHFAKSDRIEPLTFYLDPQWQLALNPSERKYCGSGFHGSDNVFSNMQALFVGYGPGFK | |
| | HGIEADTFENIEVYNLMCDLLNLTPAPNNGTHGSLNHLLKNPVYTPKHPKEVHPLVQC | |
| | PFTRNPRDNLGCSCNPSILPIEDFQTQFNLTVAEEKIIKHETLPYGRPRVLQKENTIC | |
| | LLSQHQFMSGYSQDILMPLWTSYTVDRNDSFSTEDFSNCLYQDFRIPLSPVHKCSFYK | |
| | NNTKVSYGFLSPPQLNKNSSGIYSEALLTTNIVPMYQSFQVIWRYFHDTLLRKYAEER | |
| | NGVNVVSGPVFDFDYDGRCDSLENLRQKRRVIRNQEILIPTHFFIVLTSCKDTSQTPL | |
| | HCENLDTLAFILPHRTDNSESCVHGKHDSSWVEELLMLHRARITDVEHITGLSFYQQR | |
| | KEPVSDILKLKTHLPTFSQED | |
| 14 | GLKPSCAKEVKSCKGRCFERTFGNCRCDAACVELGNCCLDYQETCIEPEHIWTCNKER | Exemplary |
| | CGEKRLTRSLCACSDDCKDKGDCCINYSSVCQGEKSWVEEPCESINEPQCPAGFETPP | soluble form of |
| | TLLFSLDGFRAEYLHTWGGLLPVISKLKKCGTYTKNMRPVYPTKTFPNHYSIVTGLYP | ENPP1 |
| | ESHGIIDNKMYDPKMNASFSLKSKEKENPEWYKGEPIWVTAKYQGLKSGTFFWPGSDV | |
| | EINGIFPDIYKMYNGSVPFEERILAVLQWLQLPKDERPHFYTLYLEEPDSSGHSYGPV | |
| | SSEVIKALQRVDGMVGMLMDGLKELNLHRCLNLILISDHGMEQGSCKKYIYLNKYLGD | |
| | VKNIKVIYGPAARLRPSDVPDKYYSFNYEGIARNLSCREPNQHFKPYLKHFLPKRLHF | |
| | AKSDRIEPLTFYLDPQWQLALNPSERKYCGSGFHGSDNVFSNMQALFVGYGPGFKHGI | |
| | EADTFENIEVYNLMCDLLNLTPAPNNGTHGSLNHLLKNPVYTPKHPKEVHPLVQCPFT | |
| | RNPRDNLGCSCNPSILPIEDFQTQFNLTVAEEKIIKHETLPYGRPRVLQKENTICLLS | |
| | QHQFMSGYSQDILMPLWTSYTVDRNDSFSTEDFSNCLYQDFRIPLSPVHKCSFYKNNT | |
| | KVSYGFLSPPQLNKNSSGIYSEALLTTNIVPMYQSFQVIWRYFHDTLLRKYAEERNGV | |
| | NVVSGPVFDFDYDGRCDSLENLRQKRRVIRNQEILIPTHFFIVLTSCKDTSQTPLHCE | |
| | NLDTLAFILPHRTDNSESCVHGKHDSSWVEELLMLHRARITDVEHITGLSFYQQRKEP | |
| | VSDILKLKTHLPTFSQED | |
| 15 | PSCAKEVKSCKGRCFERTFGNCRCDAACVELGNCCLDYQETCIEPEHIWTCNKFRCGE | Exemplary |
| | KRLTRSLCACSDDCKDKGDCCINYSSVCQGEKSWVEEPCESINEPQCPAGFETPPTLL | soluble form of |
| | FSLDGFRAEYLHTWGGLLPVISKLKKCGTYTKNMRPVYPTKTFPNHYSIVTGLYPESH | ENPP1 |
| | GIIDNKMYDPKMNASFSLKSKEKENPEWYKGEPIWVTAKYQGLKSGTFFWPGSDVEIN | |
| | GIFPDIYKMYNGSVPFEERILAVLQWLQLPKDERPHFYTLYLEEPDSSGHSYGPVSSE | |
| | VIKALQRVDGMVGMLMDGLKELNLHRCLNLILISDHGMEQGSCKKYIYLNKYLGDVKN | |
| | IKVIYGPAARLRPSDVPDKYYSFNYEGIARNLSCREPNQHFKPYLKHFLPKRLHFAKS | |
| | DRIEPLTFYLDPQWQLALNPSERKYCGSGFHGSDNVFSNMQALFVGYGPGFKHGIEAD | |
| | TFENIEVYNLMCDLLNLTPAPNNGTHGSLNHLLKNPVYTPKHPKEVHPLVQCPFTRNP | |
| | RDNLGCSCNPSILPIEDFQTQFNLTVAEEKIIKHETLPYGRPRVLQKENTICLLSQHQ | |
| | FMSGYSQDILMPLWTSYTVDRNDSFSTEDFSNCLYQDFRIPLSPVHKCSFYKNNTKVS | |
| | YGFLSPPQLNKNSSGIYSEALLTTNIVPMYQSFQVIWRYFHDTLLRKYAEERNGVNVV | |
| | SGPVFDFDYDGRCDSLENLRQKRRVIRNQEILIPTHFFIVLTSCKDTSQTPLHCENLD | |
| | TLAFILPHRTDNSESCVHGKHDSSWVEELLMLHRARITDVEHITGLSFYQQRKEPVSD | |
| | ILKLKTHLPTFSQED | |

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Numeric ranges disclosed herein are inclusive of the numbers defining the ranges.

A polypeptide disclosed herein can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting (e.g., naturally-occurring or wild-type) polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### EXEMPLIFICATION

The invention now being generally described, it will be more readily understood by reference to the following examples, which are included merely for purposes of illustration of certain embodiments and embodiments of the present invention and are not intended to limit the invention.

### Example 1. Generation of ENPP1 Fusion Proteins

A soluble ENPP1 fusion protein was fused to a human Fc domain with a linker via a linker (comprising a leucine, isoleucine, and asparagine), hereinafter referred to as ENPP1-Fc. Three ENPP1-Fc constructs are shown in Table 2 as SEQ ID NOs: 9, 10, and 11 as purified from CHO cells.

Purification of ENPP1-Fc could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. Following purification of the protein, the catalytic activity of the ENPP1-Fc protein could be evaluated using pNP-TMP as a chromogenic substrate. In order to identify formulations that would result in optimal chemical, physical and structural stability of ENPP1-Fc in under both stressed and non-stressed conditions, multiple buffer types, pH conditions, excipients, and surfactant concentrations were evaluated in an iterative fashion over the course of a baseline buffer, additive, solubility, surfactant screen, and a cofactor screen.

### Example 2. Generation of Lyophilized ENPP1 Polypeptide Formulations

Lyophilized ENPP1 polypeptide formulations disclosed herein were generated according to the following steps. ENPP1-Fc at a concentration of 44 mg/mL was buffer exchanged into a formulation buffer lacking surfactant. The buffer-exchange process was performed for a total of four cycles and a total dilution of >400-fold. The protein concentration in the buffer exchanged samples was measured by UV-Visible spectroscopy using an extinction coefficient of 1.47 ml/mg*cm and a path length of 1.0 cm. During normalization to 50.0 mg/mL, surfactant (PS20) was added to all formulations to a final concentration of 0.05%. Following buffer-exchange, the formulations were sterile filtered, filled into triple-rinsed, autoclaved vials with a fill volume of 0.5 mL, and fitted with sterilized rubber stoppers.

The ENPP1-Fc lyophilization cycle (Table 3) consisted of an initial annealing step, where the temperature was cycled from −50° C. to −20° C. for a several hours to allow for complete crystallization of the mannitol containing formulations. Subsequent to the annealing step, primary drying was initiated by setting the pressure at 100 m Torr and increasing the shelf temperature to 25° C., the cycle was held at primary drying for 1793 min (about 30 hrs). After primary drying was completed, a secondary drying to remove any remaining moisture was performed by increasing the temperature to 40° C. and holding for 600 min (10 hrs). The total duration of the lyophilization cycle was 3648 min (about 2.5 days). At the completion of the cycle, vials were back filled with nitrogen at a partial pressure of about 570 Torrs, stoppered and appropriately sealed. All lyophilized cakes containing ENPP1-Fc showed intact structures of ivory color. After reconstitution, liquid appearance was observed to be brownish-yellow, and clear and free of visible particulates for all sample types and conditions. In order to identify formulations that would result in optimal chemical, physical and structural stability of ENPP1-Fc under both stressed and non-stressed conditions, multiple buffers, pH conditions, excipients, additives, surfactants, and cofactors were evaluated in an iterative fashion.

TABLE 3
ENPP1-Fc Lyophilization Cycle

| | Temp | Rate | Ramp Time | Hold Time | | | | |
|---|---|---|---|---|---|---|---|---|
| | (° C.) | (C/min) | (min) | (min) | Pressure | Total time | | |
| Load at RT | 25° C. | | | 10 min | | | | |
| Ramp | −5° C. | 0.5° C./min | 60 min | | | 70 min | 1.2 hrs | 0.05 days |
| Hold | −5° C. | | | 60 min | | 130 min | 2.2 hrs | 0.09 days |
| Ramp | −50° C. | 0.5° C./min | 90 min | | | 220 min | 3.7 hrs | 0.15 days |
| Hold | −50° C. | | | 180 min | | 400 min | 6.7 hrs | 0.28 days |
| Ramp | −20° C. | 0.5° C./min | 60 min | | | 460 min | 7.7 hrs | 0.32 days |
| Hold | −20° C. | | | 180 min | | 640 min | 10.7 hrs | 0.44 days |
| Ramp | −50° C. | 0.5° C./min | 60 min | | | 700 min | 11.7 hrs | 0.49 days |
| Hold | −50° C. | | | 180 min | | 880 min | 14.7 hrs | 0.61 days |
| Vacuum start | −50° C. | | | | 100 mTorr | 880 min | 14.7 hrs | 0.61 days |
| Ramp | −25° C. | 0.5° C./min | 50 min | | 100 mTorr | 930 min | 15.5 hrs | 0.65 days |
| Primary | −25° C. | | | 1793 min | 100 mTorr | 2723 min | 45.4 hrs | 1.89 days |
| Ramp | −40° C. | 0.2° C./min | 325 min | | 100 mTorr | 3048 min | 50.8 hrs | 2.12 days |
| Secondary | −40° C. | | | 600 min | 100 mTorr | 3648 min | 60.8 hrs | 2.53 days |

### Example 3. Reconstitution of Lyophilized ENPP1 Polypeptide Formulations

lyophilized samples reconstitution was evaluated based on reconstitution with 0.5 mL of water for injection. Time was measured until all particulate matter was dissolved. Each lyophilized sample dissolved completely leaving no visible residue or undissolved matter within a period of at least 46 seconds. Summary reconstitution data are shown in Table 4.

TABLE 4
Reconstitution times for lyophilized
ENPP1-polypeptide formulations

| | | | | | | | Recon- |
|---|---|---|---|---|---|---|---|
| | | Formu- | Time | | Condi- | | struction |
| Buffer | Excipient | lation | Point | | tion | | Time (sec) |
| 20 mM | Sucrose | A | T 0 | | Lyo | | 76 |
| Succinate, | (9%) | | 4 | weeks | 5° | C. | 53 |
| 2 mM CaCl₂ | | | | | 40° | C. | 76 |
| pH 6.0 | | | 13 | weeks | 5° | C. | 52 |
| | Sucrose | B | T 0 | | Lyo | | 60 |
| | (3%) | | 4 | weeks | 5° | C. | 88 |
| | Mannitol | | | | 40° | C. | 83 |
| | (1.5%) | | 13 | weeks | 5° | C. | 46 |
| | Sucrose | C | T 0 | | Lyo | | 73 |
| | (2%) | | 4 | weeks | 5° | C. | 91 |
| | Mannitol | | | | 40° | C. | 95 |
| | (2%) | | 13 | weeks | 5° | C. | 60 |
| 20 mM | Sucrose | D | T 0 | | Lyo | | 71 |
| Citrate, | (9%) | | 4 | weeks | 5° | C. | 62 |
| 2 mM CaCl₂ | | | | | 40° | C. | 77 |
| pH 6.0 | | | 13 | weeks | 5° | C. | 92 |
| | Sucrose | E | T 0 | | Lyo | | 47 |
| | (3%) | | 4 | weeks | 5° | C. | 69 |
| | Mannitol | | | | 40° | C. | 68 |
| | (1.5%) | | 13 | weeks | 5° | C. | 54 |
| | Sucrose | F | T 0 | | Lyo | | 87 |
| | (2%) | | 4 | weeks | 5° | C. | 82 |
| | Mannitol | | | | 40° | C. | 85 |
| | (2%) | | 13 | weeks | 5° | C. | 88 |

### Example 4. Evaluation of Residual Moisture in Lyophilized ENPP1 Polypeptide Formulations

The residual moisture was evaluated for lyophilized cakes. All lyophilized samples possessed very low or no moisture content across time points and conditions. Residual moisture content ranged from 0.0%-0.3%. Summary data are shown in Table 5.

TABLE 5
Residual moisture content of lyophilized samples

| | | | | | | | Mois- |
|---|---|---|---|---|---|---|---|
| | | Formu- | Time | | Condi- | | ture |
| Buffer | Excipient | lation | Point | | tion | | (%) |
| 20 mM | Sucrose (9%) | A | T 0 | | | | 0.1 |
| Succinate, | | | 4 | weeks | 5° | C. | 0.1 |
| 2 mM CaCl₂ | | | | | 40° | C. | 0.2 |
| pH 6.0 | | | 13 | weeks | 5° | C. | 0.2 |
| | Sucrose (3%) | B | T 0 | | Lyo | | 0.0 |
| | Mannitol | | 4 | weeks | 5° | C. | 0.1 |
| | (1.5%) | | | | 40° | C. | 0.2 |
| | | | 13 | weeks | 5° | C. | 0.0 |
| | Sucrose (2%) | C | T 0 | | | | 0.1 |
| | Mannitol | | 4 | weeks | 5° | C. | 0.1 |
| | (2%) | | | | 40° | C. | 0.2 |
| | | | 13 | weeks | 5° | C. | 0.0 |
| 20 mM | Sucrose (9%) | D | T 0 | | | | 0.3 |
| Citrate, | | | 4 | weeks | 5° | C. | 0.1 |
| 2 mM CaCl₂ | | | | | 40° | C. | 0.2 |
| pH 6.0 | | | 13 | weeks | 5° | C. | 0.1 |
| | Sucrose (3%) | E | T 0 | | | | 0.1 |
| | Mannitol | | 4 | weeks | 5° | C. | 0.1 |
| | (1.5%) | | | | 40° | C. | 0.2 |
| | | | 13 | weeks | 5° | C. | 0.0 |
| | Sucrose (2%) | F | T 0 | | | | 0.0 |
| | Mannitol | | 4 | weeks | 5° | C. | 0.1 |
| | (2%) | | | | 40° | C. | 0.1 |
| | | | 13 | weeks | 5° | C. | 0.0 |

### Example 5: Baseline Buffer Evaluation

The stability of ENPP1 polypeptide formulations was evaluated over a pH range of 5.0-8.0 using a panel of candidate buffers including citrate, histidine, phosphate, acetate, sodium bicarbonate, succinate, glycylglycine, and tris. To evaluate the self-buffering capacity of these formulations, sample was buffer exchanged into water. For this study, ENPP1-Fc was buffer-exchanged into 20 mM acetate (pH 5.0), succinate (pH 5.0 and 6.0), citrate (pH 5.0, 6.0, and 7.0), sodium bicarbonate (pH 6.0 and 7.0), histidine (pH 6.0 and 7.0), potassium phosphate (pH 7.0 and 8.0), tris (pH 7.0 and 8.0) and glycylglycine (pH 8.0) buffers. ENPP1-Fc was buffer exchanged into one of the above-identified formulation buffers and concentrated to 2 mg/mL in 0.5 mL. Protein recoveries following concentration and buffer exchange were generally comparable across formulations, ranging from ˜70-100%, with the exception of ENPP1-Fc buffer exchanged into water. Buffer exchanged ENPP1-Fc in water exhibited a 34% recovery, indicating that ENPP1-Fc lacks self-buffering capacity.

The thermal stability of ENPP1 polypeptide formulations was monitored by differential scanning fluorimetry (DSF), which provides data pertaining to the melting temperature (Tm) of the polypeptide. Tm data was collected for protein samples at 2 mg/mL. Samples were equilibrated at 20° C. for 30 seconds, and the barycentric mean (BCM) of the intrinsic fluorescence spectra from 250-500 nm (266 nm excitation wavelength) was monitored while temperature increased to 95° C. at a rate of 0.5° C./minute. The inflection point of the BCM versus temperature curve during an unfolding event (identified by the maximum or minimum of the derivative trace) was identified as the Tm of that transition. Static light scattering (SLS) intensity at 266 nm and 473 nm was also measured in parallel with DSF measurements to observe the onset temperature of small and large aggregate formation (Tagg), respectively. Relative thermal stability was impacted both by buffer type and formulation pH. Citrate pH 7.0, phosphate pH 7.0/8.0, sodium bicarbonate and glycylglycine samples did not show significant light scattering at 266 nm or 473 nm, indicating an apparent lack of aggregation for these formulations. Acetate, succinate pH 5.0, citrate pH 5.0 and tris pH 7.0 formulations exhibited the highest Taggs across formulations. For citrate, histidine and tris samples, Taggs decreased with increasing pH, Taggs for phosphate appeared insensitive to change in pH. Tm values surprisingly showed a clear trend with respect to buffer type for each pH, indicating greater thermal stability for succinate at pH 5.0 and pH 6.0, and for phosphate at pH 5.0 and pH 6.0. Summary DSF data and are shown in Table 6.

TABLE 6
Summary of Baseline Buffer DFS/SLS Data

| | | | T_{agg} | T_{agg} | T_{on set} | Tₘ₁ |
|---|---|---|---|---|---|---|
| | | For- | 266 nm | 473 nm | BCM | BCM |
| Buffer | pH | mulation | (° C.) | (° C.) | (° C.) | (° C.) |
| Self (water) | N/A | A | 62.4 | 62.1 | 43.9 | 55.4 |
| 20 mM Acetate | pH 5.0 | B | 80.6 | 80.2 | 39.7 | 54.6 |
| 20 mM Succinate | pH 5.0 | C | 77.7 | 79.5 | 40.5 | 57.4 |
| | pH 6.0 | D | 65.7 | 66.2 | 53.5 | 62.8 |
| 20 mM Citrate | pH 5.0 | E | 81.0 | 80.8 | 45.7 | 57.1 |
| | pH 6.0 | F | 61.2 | 61.3 | 51.2 | 60.9 |
| | pH 7.0 | G | 58.4 | 61.7 | 49.7 | 60.5 |
| 20 mM Sodium | pH 6.0 | H | ND | ND | 45.4 | 57.0 |
| Bicarbonate | pH 7.0 | I | ND | ND | 44.7 | 56.6 |
| 20 mM Histidine | pH 6.0 | J | 66.9 | 66.5 | 40.1 | 54.3 |
| | pH 7.0 | K | 60.7 | 62.0 | 27.4 | 54.6 |
| 20 mM Potassium | pH 7.0 | L | 61.0 | 63.6 | 51.1 | 60.8 |
| Phosphate | pH 8.0 | M | 62.5 | 64.0 | 54.4 | 63.5 |
| 20 mM Tris | pH 7.0 | N | 76.6 | 79.9 | 51.3 | 59.7 |
| | pH 8.0 | O | 58.8 | 61.4 | 49.7 | 58.6 |
| 20 mM | pH 8.0 | P | 58.4 | 61.2 | 48.0 | 57.2 |
| Glycylglycine | | | | | | |

ND: Values not determined due to very low counts.

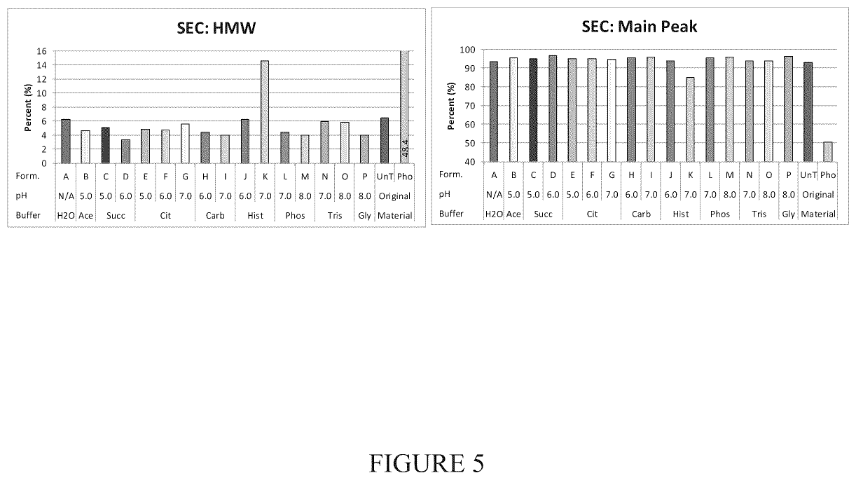

Size exclusion chromatography (SEC) used to evaluate the quantity of aggregates and degradation products present in ENPP1 polypeptide formulation samples. Percent abundances for total high molecular weight (HMW) species, low molecular weight (LMW) species, and main peak purity are reported. SEC results are summarized in Table 7 and FIG. 5. Prior to analysis by SEC, all samples were incubated at 30° C. for 48 hrs. HMW species were present in all formulations, ranging from 3.3-14.5%. The lowest levels of HMW species were observed for sodium bicarbonate and phosphate formulations. Higher-order HMW species was the highest in histidine pH 7.0.

TABLE 7
Summary Baseline Buffer Evaluation Size Exclusion
Chromatography Data

| | | | | | | Column | Total |
|---|---|---|---|---|---|---|---|
| | | | % | % | % | Recovery | Area |
| Buffer | pH | Form. | HMW | Main | LMW | (%)^{a} | (μV*s) |
| Self (water) | 0 | A | 6.2 | 93.3 | 0.5 | 94 | 8691405 |
| 20 mM Acetate | 5.0 | B | 4.6 | 95.4 | ND | 86 | 16192216 |
| 20 mM | 5.0 | C | 5.1 | 94.9 | ND | 91 | 17757935 |
| Succinate | 6.0 | D | 3.3 | 96.5 | 0.3 | 99 | 20957945 |
| 20 mM Citrate | 5.0 | E | 4.8 | 95.2 | ND | 94 | 19085508 |
| | 6.0 | F | 4.7 | 95.1 | 0.2 | 97 | 19232879 |
| | 7.0 | G | 5.5 | 94.5 | ND | 97 | 19272431 |
| 20 mM Sodium | 6.0 | H | 4.4 | 95.3 | 0.3 | 98 | 19623052 |
| Bicarbonate | 7.0 | I | 3.9 | 95.7 | 0.3 | 97 | 20538912 |
| 20 mM | 6.0 | J | 6.2 | 93.8 | ND | 95 | 20688528 |
| Histidine | 7.0 | K | 14.5 | 85.0 | 0.5 | 95 | 16752179 |
| 20 mM | 7.0 | L | 4.4 | 95.4 | 0.2 | 97 | 18897632 |
| Potassium | 8.0 | M | 4.0 | 95.8 | 0.2 | 98 | 19646363 |
| Phosphate | | | | | | | |
| 20 mM Tris | 7.0 | N | 5.9 | 93.9 | 0.3 | 96 | 19059185 |
| | 8.0 | O | 5.7 | 93.9 | 0.4 | 98 | 18835534 |
| 20 mM | 8.0 | P | 4.0 | 96.0 | ND | 98 | 19571596 |
| Glycylglycine | | | | | | | |

| | | | | | |
|---|---|---|---|---|---|
| CC-15 Starting Material | 6.4 | 92.9 | 0.7 | 98 | 18917341 |
| CC-15 Photo Stressed | 48.4 | 50.5 | 1.1 | 101 | 19483433 |

ªColumn Recovery (%) = ((Total Peak Area/10\hat{ }6)*(Flow Rate/60)(Extinction coeff.*Path Length))/(Sample Concentration*Injection Volume/10\hat{ }3)*100

In summary, the baseline buffer evaluation data indicate buffer formulations comprising ≤pH 5.0, and ≥pH 6.0 provide the greatest stability for the lyophilized ENPP1 polypeptide formulation. Histidine pH 7.0 is less preferred than the other buffer systems tested. Acetate is incapable of buffering the lyophilized formulation in the necessary range. Based on the above data, buffers were binned into two regimes comprising those which support a pH range from pH 6 to pH 7 (succinate, citrate, bicarbonate) and those which support a pH range of pH 7 to pH 8 (phosphate, tris, glycylglycine). In the first buffer regime, citrate formulations demonstrated superior Tonset (on the order of 5° C.). In the second buffer regime, tris formulations demonstrated superior Tonset (on the order of 2° C.). The chosen pH levels for further formulation development were pH 6.0, pH 7.0 and pH 8.0, using succinate, citrate and phosphate buffer systems, respectively. At each buffer type, five different pharmaceutically acceptable additives were evaluated: arginine, NaCl, sucrose, mannitol, and glycine. The additives proposed cover a range of molecule classes (i.e. amino acid, salt, sugar, and polyol).

### Example 6: Pharmaceutically Acceptable Additive Screen

The stability of lyophilized ENPP1 polypeptide formulations was evaluated in the presence of various additives with reported stabilizing properties at roughly isotonic concentrations, using the buffering systems defined in the baseline buffer evaluation (20 mM succinate, pH 6.0; citrate, pH 7.0, and phosphate, pH 8.0). ENPP1-Fc was buffer-exchanged into each candidate buffer containing either 150 mM NaCl, 150 mM L-arginine, 250 mM sucrose, 250 mM mannitol or 200 mM glycine.

DSF/SLS data for formulations of ENPP1-Fc containing additives indicated that the additives did not have major effects upon thermal stability as evaluated by Tm (DSF) and Tagg at both 266 and 473 nm (SLS). Overall, DSF data showed comparable melting temperatures and an apparent lack of larger aggregates for the majority of the formulations.

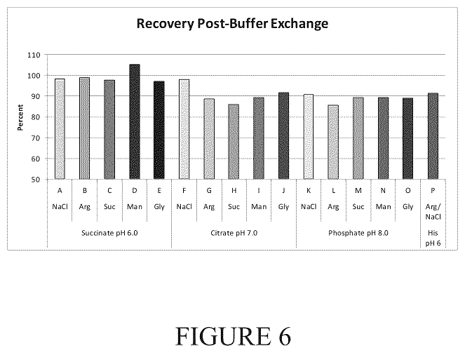

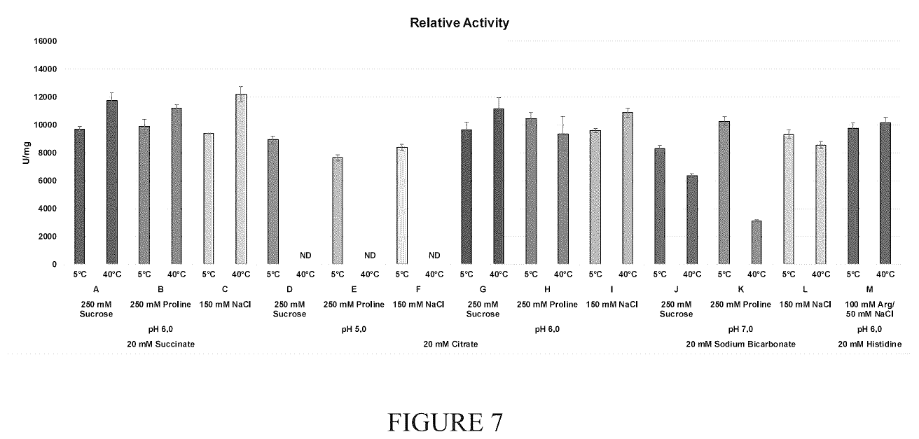

SEC data (summarized in Table 8 and FIG. 6) indicated that glycine and NaCl/arginine combination produced the highest % HMW species across formulations. NaCl at pH 6.0 and pH 7.0, and arginine pH 6.0 behaved similarly to sucrose. Sucrose generated the least % HMW species (≤5.3%) across each of the tested formulations. The SEC data demonstrates that formulations containing sucrose provided the highest overall stability. Table 8: SEC Additive Screen Summary Data

| | | | | | | Column | Total |
|---|---|---|---|---|---|---|---|
| | | | % | % | % | Recovery | Area |
| Buffer | Excipient | Form. | HMW | Main | LMW | (%)^{a} | (μV*s) |
| Succinate | NaCl | A | 5.3 | 94.4 | 0.3 | 99 | 19084426 |
| pH 6.0 | Arginine | B | 4.7 | 94.9 | 0.3 | 98 | 18440146 |
| | Sucrose | C | 4.6 | 94.9 | 0.5 | 99 | 18651298 |
| | Mannitol | D | 5.1 | 94.3 | 0.6 | 98 | 19005559 |
| | Glycine | E | 7.1 | 92.6 | 0.3 | 98 | 18936695 |
| Citrate | NaCl | F | 5.7 | 93.9 | 0.4 | 97 | 18937420 |
| pH 7.0 | Arginine | G | 8.4 | 91.1 | 0.4 | 96 | 18724123 |
| | Sucrose | H | 5.2 | 94.4 | 0.4 | 97 | 19198190 |
| | Mannitol | I | 5.9 | 93.7 | 0.4 | 98 | 19285938 |
| | Glycine | J | 10.4 | 89.3 | 0.4 | 96 | 18259087 |
| Phosphate | NaCl | K | 7.1 | 92.7 | 0.2 | 98 | 19447707 |
| pH 8.0 | Arginine | L | 6.8 | 92.9 | 0.3 | 97 | 19095422 |
| | Sucrose | M | 5.3 | 94.4 | 0.2 | 99 | 19455680 |
| | Mannitol | N | 8.1 | 91.7 | 0.2 | 98 | 19564507 |
| | Glycine | O | 14.9 | 84.8 | 0.3 | 96 | 18730132 |
| Histidine | Arginine/ | P | 15.7 | 83.8 | 0.4 | 93 | 18378845 |
| pH 6.0 | NaCl | | | | | | |

| | | | | | |
|---|---|---|---|---|---|
| Starting Material | 8.1 | 91.5 | 0.4 | 96 | 18756246 |

^{a}Column Recovery (%) = ((Total Peak Area/10\hat{ }6)*(Flow Rate/60)(Extinction coeff.*Path Length))/(Sample Concentration*Injection Volume/10\hat{ }3)*100

### Example 7: Solubility Screens for Lyophilyzed ENPP1 Polypeptide Formulations

In order to establish conditions for maximum solubility of the lyophilized ENPP1 polypeptide formulation, 20 mM succinate pH 6.0, citrate pH 5.0 and pH 6.0, sodium bicarbonate pH 7.0 and histidine pH 6.0 were used in the presence of isotonic concentrations of sucrose, proline and NaCl. After buffer exchange and concentration, samples were split into two equal volume aliquots, one of the aliquots was heat stressed at 40° C. for ˜7 days and the other was retained at 5° C. Heat-stressed and 5° C. samples were evaluated for enzymatic activity, and for formation of HMW species by SEC. The enzymatic assay used to evaluate the activity of each sample was performed by mixing ENPP1-Fc with pNP-TMP. Product formation (pNP) is monitored spectrophotometrically at 405 nm for a period of 5 minutes, at 25° C. Specific activity of ENPP1-Fc is calculated by interpolating the change in absorbance over time obtained for each sample preparation to a standard curve of known concentrations of pNP. Results are reported relative to reference sample material, where units corresponds to moles of product generated over time (min). Solubility screen enzymatic activity assay results are shown in Table 9 and FIG. 7. Formulations comprising pH 6.0 demonstrated increased thermal stability, as noted in the heightened enzymatic activity observed for each of these samples. Values for citrate at pH 5.0, incubated at 40° C. were not determined due to jellification of the samples. Overall, the enzymatic activity suggests that succinate and citrate at pH 6.0 are the best performing buffer types across evaluated additives when comparing 5° C. versus 40° C. incubated samples.

TABLE 9
Summary Enzymatic Activity Assay Solubility Screen Data

| | | | For- | | | |
|---|---|---|---|---|---|---|
| | | | mula- | Con- | | |
| Buffer | pH | Excipient | tion | dition | U/mL | U/mg |
| 20 mM | pH 6.0 | 250 mM Sucrose | A | 5° C. | 9.8 | 9668 |
| Succinate | | | | 40° C. | 11.8 | 11721 |
| | | 250 mM Proline | B | 5° C. | 10.5 | 9878 |
| | | | | 40° C. | 11.9 | 11156 |
| | | 150 mM NaCl | C | 5° C. | 9.2 | 9363 |
| | | | | 40° C. | 12.0 | 12180 |
| 20 mM | pH 5.0 | 250 mM Sucrose | D | 5° C. | 8.9 | 8905 |
| Citrate | | | | 40° C. | ND | ND |
| | | 250 mM Proline | E | 5° C. | 7.9 | 7607 |
| | | | | 40° C. | ND | ND |
| | | 150 mM NaCl | F | 5° C. | 8.7 | 8365 |
| | | | | 40° C. | ND | ND |
| | pH 6.0 | 250 mM Sucrose | G | 5° C. | 10.1 | 9612 |
| | | | | 40° C. | 11.7 | 11127 |
| | | 250 mM Proline | H | 5° C. | 10.6 | 10409 |
| | | | | 40° C. | 9.5 | 9349 |
| | | 150 mM NaCl | I | 5° C. | 9.7 | 9582 |
| | | | | 40° C. | 11.0 | 10863 |
| 20 mM | pH 7.0 | 250 mM Sucrose | J | 5° C. | 8.6 | 8291 |
| Sodium | | | | 40° C. | 6.5 | 6309 |
| Bicarbonate | | 250 mM Proline | K | 5° C. | 10.5 | 10249 |
| | | | | 40° C. | 3.2 | 3107 |
| | | 150 mM NaCl | L | 5° C. | 9.7 | 9300 |
| | | | | 40° C. | 8.9 | 8528 |
| 20 mM | pH 6.0 | 100 mM Arg/50 | M | 5° C. | 9.6 | 9704 |
| Histidine | | mM NaCl | | 40° C. | 10.0 | 10113 |

| | | |
|---|---|---|
| Starting Material | 10.3 | 10459 |

ND: Not Determined

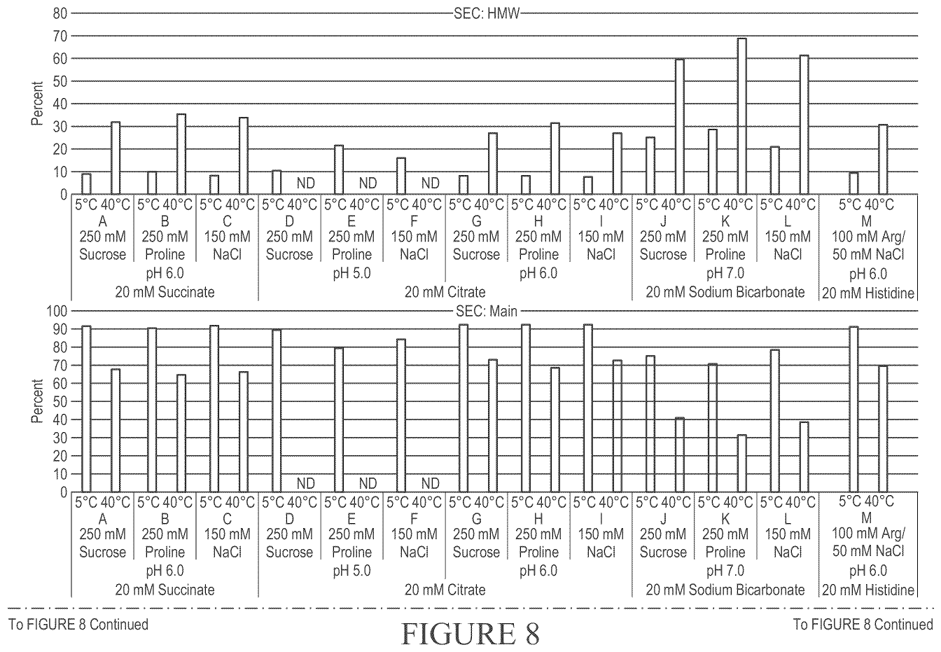

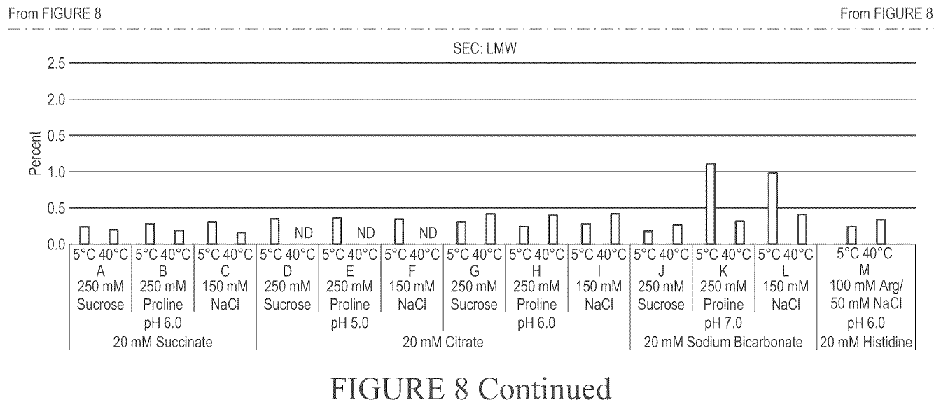

The formation of HMW species as determined by SEC was consistent with the trends observed in the enzymatic activity screen. Succinate and citrate at pH 6.0 exhibited the lowest % HMW, independently of the additive used in the formulation. Citrate pH 6.0 at 40° C. was superior to all other 40° C. samples. Proline and NaCl in citrate pH 5.0, and sodium bicarbonate pH 7.0 generated the overall highest % HMW species. Summary SEC solubility screen data are shown in Table 10 and FIG. 8.

TABLE 10
Summary SEC solubility screen data

| | | | For- | | Main | | Total |
|---|---|---|---|---|---|---|---|
| | | | mula- | HMW | Peak | LMW | Area |
| Buffer | pH | Excipient | tion | (%) | (%) | (%) | (μV*s) |
| 20 mM | pH 6.0 | 250 mM | A | 6.0 | 93.8 | 0.2 | 50123877 |
| Succinate | | Sucrose | | | | | |
| | | 250 mM | B | 6.3 | 93.5 | 0.2 | 35728620 |
| | | Proline | | | | | |
| | | 150 mM | C | 5.9 | 93.8 | 0.2 | 35824216 |
| | | NaCl | | | | | |
| 20 mM | pH 5.0 | 250 mM | D | 8.2 | 91.5 | 0.3 | 39059229 |
| Citrate | | Sucrose | | | | | |
| | | 250 mM | E | 14.6 | 85.0 | 0.4 | 27138683 |
| | | Proline | | | | | |
| | | 150 mM | F | 11.7 | 88.0 | 0.3 | 28169043 |
| | pH 6.0 | 250 mM | G | 5.3 | 94.5 | 0.2 | 52451719 |
| | | Sucrose | | | | | |
| | | 250 mM | H | 5.1 | 94.7 | 0.3 | 30695670 |
| | | Proline | | | | | |
| | | 150 mM | I | 5.5 | 94.3 | 0.3 | 41407595 |
| | | NaCl | | | | | |
| 20 mM | pH 7.0 | 250 mM | J | 13.6 | 86.2 | 0.2 | 43615676 |
| Sodium | | Sucrose | | | | | |
| Bicar- | | 250 mM | K | 16.5 | 83.3 | 0.2 | 51379433 |
| bonate | | Proline | | | | | |
| | | 150 mM | L | 12.5 | 87.3 | 0.2 | 34932916 |
| | | NaCl | | | | | |
| 20 mM | pH 6.0 | 100 mM | M | 5.7 | 94.0 | 0.3 | 33330444 |
| Histidine | | Arg/50 | | | | | |
| | | mM NaCl | | | | | |

### Example 8: Surfactant Screen

In order to identify superior surfactants for use in lyophilized ENPP1 polypeptide formulations, ENPP1-Fc was buffer-exchanged into three base formulations (succinate/sucrose pH 6.0, succinate/NaCl pH 6.0 and citrate/sucrose pH 7.0) and a control formulation (histidine/arginine/NaCl pH 6.0). The formulation samples were subjected to stress via freeze-thaw cycling and mechanical stress, in addition two small aliquots were reserved as no-stress controls. Freeze thaw cycling samples were frozen at −80° C. for ≥60 minutes and thawed at room temperature. This process was repeated for a total of 5 cycles. For agitation stress, samples were placed on a microplate shaker set at ˜600 rpm, protected from light, for ˜72 hours at room temperature.

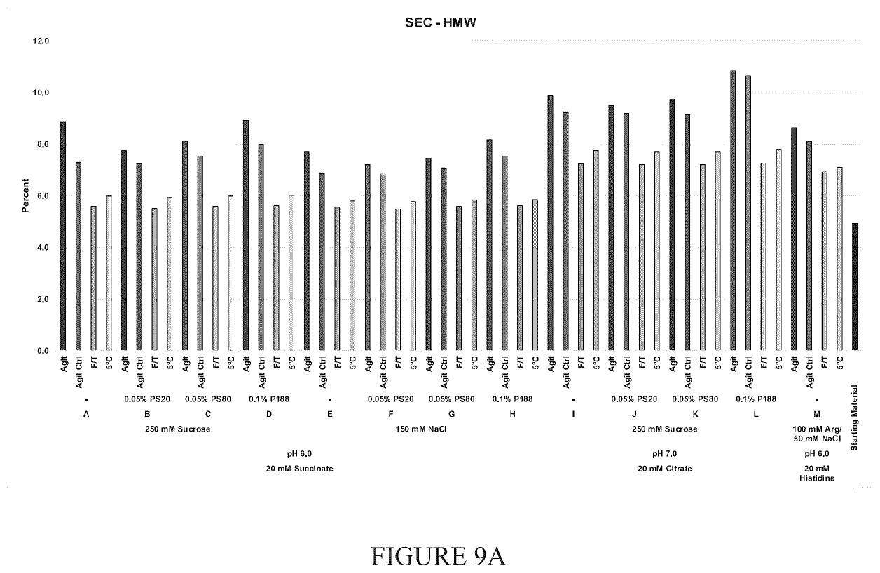

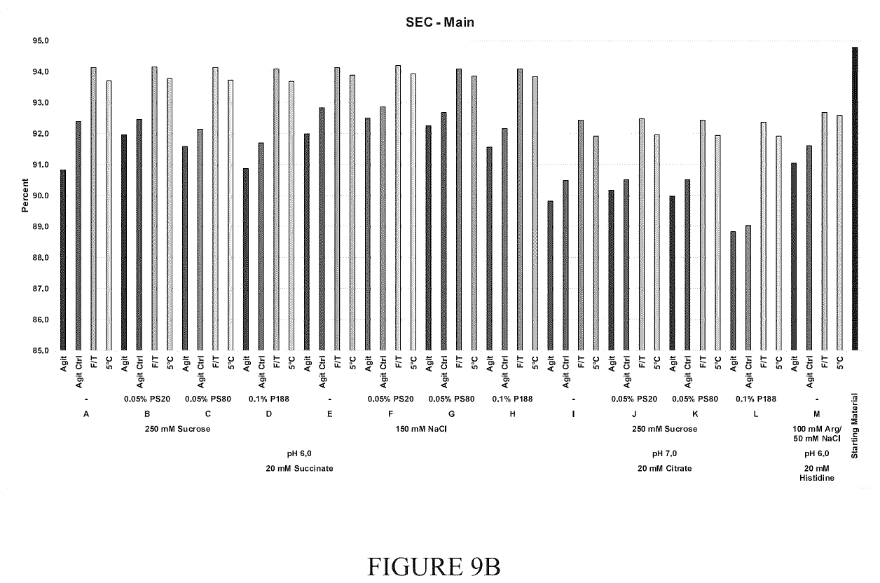

To assess stability/aggregation of the stressed samples, enzymatic activity and was measured and SEC-HPLC was performed. Enzymatic activity across surfactants for all stress conditions and formulations was comparable, indicating that the enzymatic activity of ENPP1-Fc was not impacted under the tested conditions. SEC evaluation for surfactant studies are shown in Table 11, FIG. 9A and FIG. 9B. The % HMW content across all formulations indicate that surfactants had no impact on samples submitted to freeze/thaw stress, as values have shown to be equivalent between the stressed samples and respective controls. For the agitation stressed samples, P188 samples were the least stable of the three tested surfactants. PS20 provided superior stability under agitation stress, and generated decreased % HMW content across the three tested surfactants. These data indicate that the presence and type of surfactant impact ENPP1-Fc stability following physical stress. The SEC demonstrates that PS20 is the optimal surfactant type for EnPP1-Fc stability at 50 mg/mL. After agitation, P188 showed elevated levels of total higher order aggregates in most excipient/buffer type combinations.

TABLE 11
Summary Surfactant Screen SEC data

| | | | | | | | | | | Total |
|---|---|---|---|---|---|---|---|---|---|---|
| | | | | | | % | % | 0% | Column | Area |
| Buffer | pH | Excipient | Formulation | Surfactant | Condition | HMW | Main | LMW | Recovery | (μV*s) |
| 20 mM | pH | 250 mM | A | — | Agit | 8.9 | 90.8 | 0.3 | 101 | 3480 |
| Succinate | 6.0 | Sucrose | | | Agit Ctrl | 7.3 | 92.4 | 0.3 | 101 | 3303 |
| | | | | | F/T | 5.6 | 94.1 | 0.3 | 100 | 3505 |
| | | | | | 5° C. | 6.0 | 93.7 | 0.3 | 101 | 3598 |
| | | | B | 0.05% | Agit | 7.8 | 92.0 | 0.3 | 98 | 3365 |
| | | | | PS20 | Agit Ctrl | 7.2 | 92.4 | 0.3 | 98 | 3330 |
| | | | | | F/T | 5.5 | 94.2 | 0.3 | 98 | 3510 |
| | | | | | 5° C. | 5.9 | 93.8 | 0.3 | 98 | 3634 |
| | | | C | 0.05% | Agit | 8.1 | 91.6 | 0.3 | 98 | 3415 |
| | | | | PS80 | Agit Ctrl | 7.5 | 92.1 | 0.3 | 97 | 3260 |
| | | | | | F/T | 5.6 | 94.1 | 0.3 | 99 | 3568 |
| | | | | | 5° C. | 6.0 | 93.7 | 0.3 | 97 | 3648 |
| | | | D | 0.1% | Agit | 8.9 | 90.9 | 0.3 | 87 | 3332 |
| | | | | P188 | Agit Ctrl | 8.0 | 91.7 | 0.3 | 87 | 3316 |
| | | | | | F/T | 5.6 | 94.1 | 0.3 | 87 | 3528 |
| | | | | | 5° C. | 6.0 | 93.7 | 0.3 | 88 | 3595 |
| | | 150 mM | E | — | Agit | 7.7 | 92.0 | 0.3 | 99 | 3346 |
| | | NaCl | | | Agit Ctrl | 6.9 | 92.8 | 0.3 | 98 | 3223 |
| | | | | | F/T | 5.6 | 94.1 | 0.3 | 99 | 3510 |
| | | | | | 5° C. | 5.8 | 93.9 | 0.3 | 99 | 3621 |
| | | | F | 0.05% | Agit | 7.2 | 92.5 | 0.3 | 90 | 3406 |
| | | | | PS20 | Agit Ctrl | 6.8 | 92.8 | 0.3 | 92 | 3133 |
| | | | | | F/T | 5.5 | 94.2 | 0.3 | 91 | 3494 |
| | | | | | 5° C. | 5.8 | 93.9 | 0.3 | 94 | 3596 |
| | | | G | 0.05% | Agit | 7.5 | 92.2 | 0.3 | 90 | 3433 |
| | | | | PS80 | Agit Ctrl | 7.0 | 92.7 | 0.3 | 91 | 3278 |
| | | | | | F/T | 5.6 | 94.1 | 0.3 | 90 | 3562 |
| | | | | | 5° C. | 5.8 | 93.9 | 0.3 | 90 | 3631 |
| | | | H | 0.1% | Agit | 8.2 | 91.5 | 0.3 | 92 | 3309 |
| | | | | P188 | Agit Ctrl | 7.5 | 92.1 | 0.3 | 94 | 3151 |
| | | | | | F/T | 5.6 | 94.1 | 0.3 | 95 | 3460 |
| | | | | | 5° C. | 5.9 | 93.8 | 0.3 | 95 | 3584 |
| 20 mM | pH | 250 mM | I | — | Agit | 9.9 | 89.8 | 0.3 | 87 | 3403 |
| Citrate | 7.0 | Sucrose | | | Agit Ctrl | 9.2 | 90.5 | 0.3 | 88 | 3115 |
| | | | | | F/T | 7.2 | 92.4 | 0.3 | 8 | 3454 |
| | | | | | 5° C. | 7.8 | 91.9 | 0.3 | 88 | 3598 |
| | | | J | 0.05% | Agit | 9.5 | 90.2 | 0.3 | 93 | 3339 |
| | | | | PS20 | Agit Ctrl | 9.2 | 90.5 | 0.3 | 92 | 3074 |
| | | | | | F/T | 7.2 | 92.5 | 0.3 | 93 | 3489 |
| | | | | | 5° C. | 7.7 | 92.0 | 0.4 | 93 | 3537 |
| | | | K | 0.05% | Agit | 9.7 | 90.0 | 0.3 | 101 | 3304 |
| | | | | PS80 | Agit Ctrl | 9.2 | 90.5 | 0.3 | 100 | 3081 |
| | | | | | F/T | 7.2 | 92.4 | 0.4 | 101 | 3535 |
| | | | | | 5° C. | 7.7 | 91.9 | 0.4 | 101 | 3555 |
| | | | L | 0.1% | Agit | 10.8 | 88.8 | 0.4 | 91 | 3268 |
| | | | | P188 | Agit Ctrl | 10.6 | 89.0 | 0.3 | 92 | 3063 |
| | | | | | F/T | 7.3 | 92.4 | 0.4 | 92 | 3512 |
| | | | | | 5° C. | 7.8 | 91.9 | 0.3 | 92 | 3600 |
| 20 mM | pH | 100 mM | M | — | Agit | 8.6 | 91.0 | 0.3 | 101 | 3329 |
| Histidine | 6.0 | Arg/50 | | | Agit Ctrl | 8.1 | 91.6 | 0.3 | 96 | 3112 |
| | | mM NaCl | | | F/T | 6.9 | 92.7 | 0.4 | 101 | 3468 |
| | | | | | 5° C. | 7.1 | 92.6 | 0.3 | 101 | 3560 |

| | | | | | |
|---|---|---|---|---|---|
| Starting Material | 4.9 | 94.8 | 0.3 | 102 | 3590 |

### Example 9: Cofactor Screening

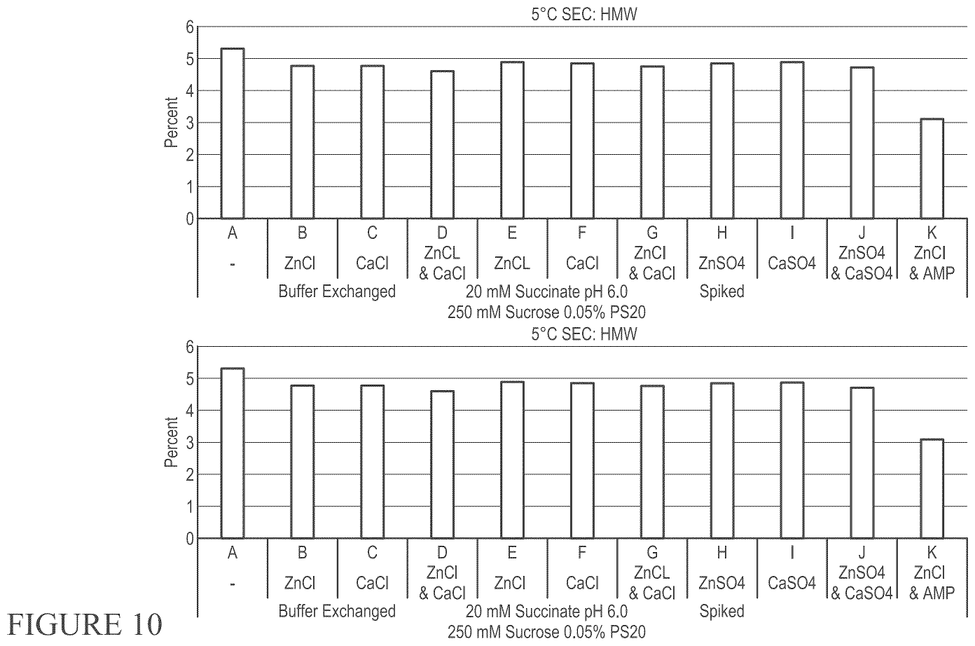

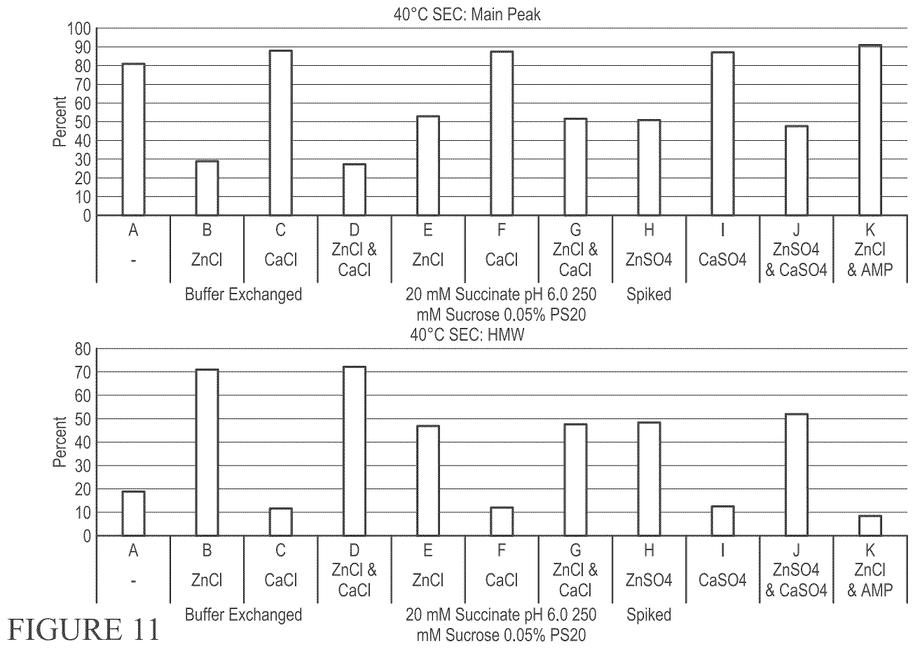

11 different cofactor combinations (including a no cofactor negative control) in 20 mM succinate, 250 mM sucrose, pH 6.0 were investigated. The tested cofactors included Zn+2 and Ca+2, with chloride and sulfate as counter ions, as well as adenosine monophosphate (AMP). SEC experiments were performed for each cofactor combination. The SEC data is summarized in Table 12. FIG. 10. These data demonstrate that AMP/ZnCl exhibited the lowest levels of % HMW. However, for cofactors tested the absence of AMP, Ca2+, either with chloride or sulfate as counter ion, produced the lowest % HMW content. The Ca2+ cofactor surprisingly stabilized ENPP1-Fc, but also demonstrated the capability of reverting higher order aggregate species potentially formed during sample preparation. These effects are observed by comparing formulations A and C (FIG. 11). Zinc produced the highest levels of % HMW content, however % HMW in the presence of zinc is significantly diminished by addition of amp, reducing % HMW levels below that of the source material used to generate all samples, formulation A. These results indicate that the presence and type of cofactor surprisingly and significantly increases the stability of ENPP1-Fc.

TABLE 12
Cofactor Screen Cofactor Combination Summary SEC Data

| | | | | | | | Column | USP | Total | |
|---|---|---|---|---|---|---|---|---|---|---|
| | | | | HMW | Main | LMW | Recovery | Plate | Area | Conc |
| Buffer | Form | Sample Type | Cofactor | (%) | (%) | (%) | (%) | Count | (μV*s) | (mg/mL) |
| 20 mM | A | Buffer- | — | 18.8 | 80.9 | 0.3 | 97 | 3607 | | 50.5 |
| Succinate | B | Exchanged | 2 mM | 71.0 | 28.8 | 0.2 | 95 | 3300 | | 49.9 |
| | | | ZnCl | | | | | | | |
| 250 mM | C | | 2 mM | 11.5 | 88.2 | 0.3 | 99 | 3523 | | 50.6 |
| Sucrose | | | CaCl | | | | | | | |
| 0.05% | D | | 2 mM | 72.5 | 27.3 | 0.2 | 96 | 3368 | | 52.1 |
| PS20 | | | ZnCl + 2 | | | | | | | |
| pH 6.0 | | | mM Cacl | | | | | | | |
| | E | Spiked | 2 mM | 46.8 | 53.0 | 0.2 | 99 | 3644 | | 50.6 |
| | | | ZnCl | | | | | | | |
| | F | Formulation | 2 mM | 12.1 | 87.4 | 0.5 | 97 | 3540 | | 51.4 |
| | | | CaCl | | | | | | | |
| | G | A | 2 mM | 47.8 | 52.0 | 0.2 | 100 | 3538 | | 51.0 |
| | | | ZnCl + 2 | | | | | | | |
| | | | mM Cacl | | | | | | | |
| | H | | 2 mM | 48.8 | 51.0 | 0.2 | 93 | 3472 | | 54.0 |
| | | | ZnSO4 | | | | | | | |
| | I | | 2 mM | 12.3 | 87.3 | 0.4 | 97 | 3489 | | 50.1 |
| | | | CaSO4 | | | | | | | |
| | J | | S mM | 52.0 | 47.9 | 0.2 | 97 | 3294 | | 50.4 |
| | | | ZnSO4 + 2 | | | | | | | |
| | | | mM | | | | | | | |
| | | | CaSO4 | | | | | | | |
| | K | | 2 mM | 8.4 | 91.2 | 0.4 | — | 3396 | | 44.0 |
| | | | ZnCl + 50 | | | | | | | |
| | | | mM AMP | | | | | | | |

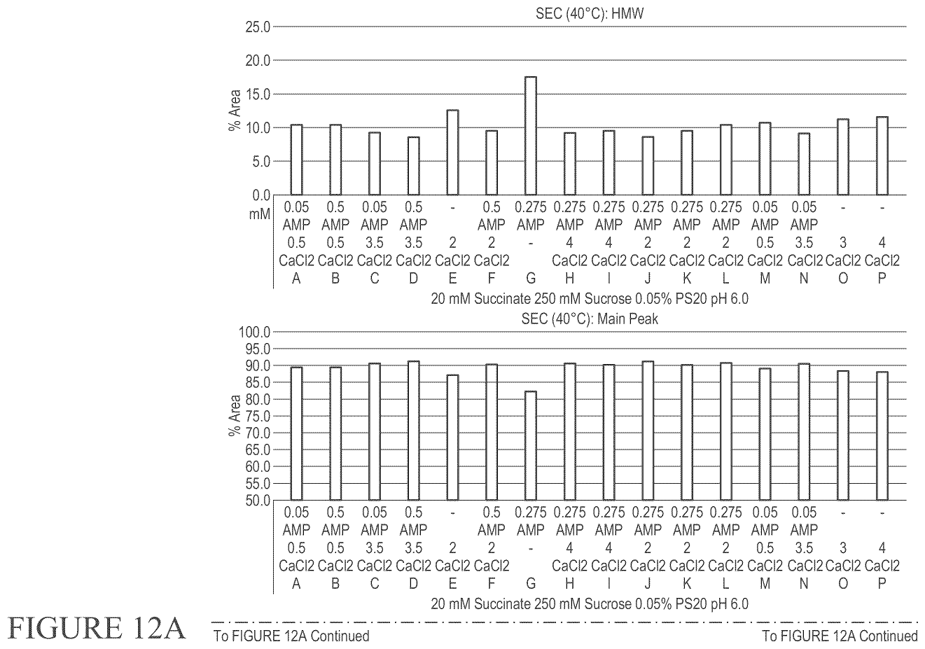

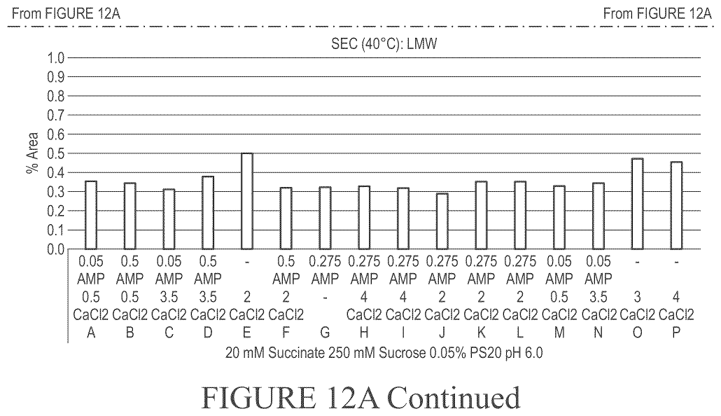

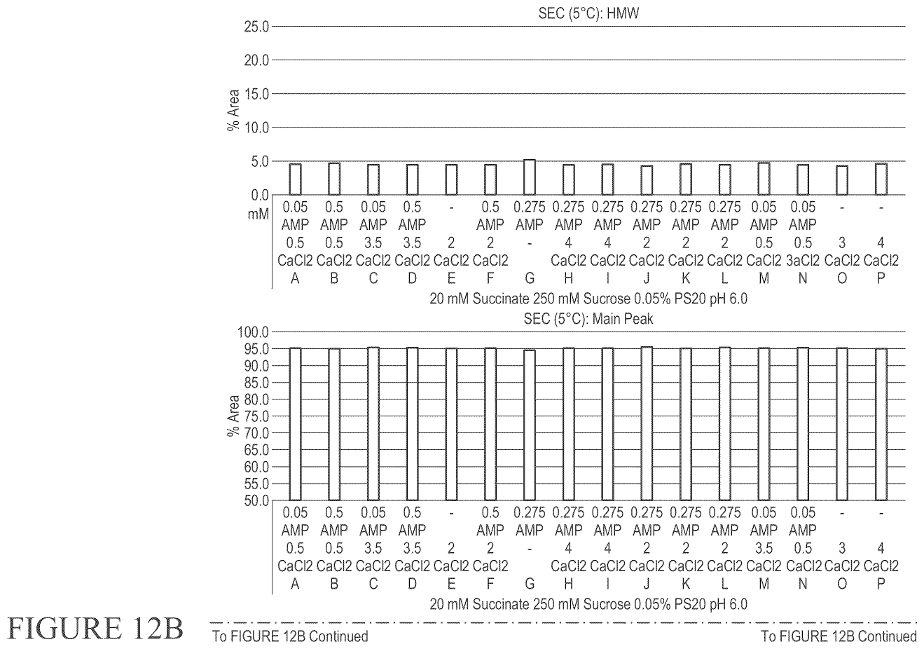

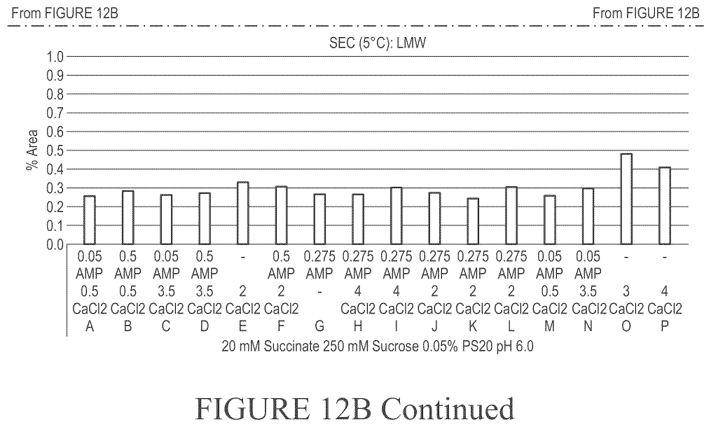

A total of 15 different buffer/cofactor combinations were further investigated. ENPP1-Fc was buffer-exchanged into 12 different formulations. The stability of the protein in the various formulations was evaluated by SEC. Prior to SEC analysis samples were stored at 40° C. for 6 days. A control sample for each formulation was kept at 5° C. for the duration of the study. The SEC data are summarized in Table 13 and FIGS. 12A-B. Consistent with the cofactor data presented above, Ca+2 and Ca+2/adenosine monophosphate generated the lowest amount of higher order aggregates across the samples that were tested. When combined with citrate and sucrose, Ca+2 and Ca+2/adenosine monophosphate each generated the lowest percentage (8.6%) of HMW, indicating that Ca+2 and Ca+2/adenosine monophosphate are superior cofactors, that when used in conjunction citrate and sucrose, surprisingly enhance the overall stability of the formulation.

TABLE 13
Cofactor Screen Buffer/Cofactor Summary SEC Data

| | | | | Main | |
|---|---|---|---|---|---|
| | | Formu- | HMW | Peak | LMW |
| Buffer | Excipient | lation | (%) | (%) | (%) |
| 20 mM Succinate, | — | A | 19.1 | 80.4 | 0.5 |
| 250 mM Sucrose | AMP (0.5 mM) | B | 18.4 | 81.2 | 0.4 |
| 0.05% PS20, | AMP (5 mM) | C | 18.7 | 80.9 | 0.4 |
| pH 6.0 | AMP (50 mM) | D | 12.4 | 86.6 | 0.9 |
| | ZnCl2 (2 mM) | E | 74.2 | 25.5 | 0.3 |
| | ZnCl2 (2 mM) + | F | 27.9 | 71.6 | 0.6 |
| | AMP (5 mM) | | | | |
| | ZnCl2 (2 mM) + | G | 9.5 | 89.7 | 0.7 |
| | AMP (50 mM) | | | | |
| | CaCl2 (2 mM) | H | 11.3 | 88.1 | 0.6 |
| | CaCl2 (2 mM) + | I | 10.2 | 89.3 | 0.5 |
| | AMP (5 mM) | | | | |
| 20 mM Citrate, | AMP (5 mM) | J | 15.3 | 84.1 | 0.5 |
| 250 mM Sucrose | CaCl2 (2 mM) | K | 12.7 | 86.4 | 0.9 |
| 0.05% PS20, | CaCl2 (2 mM) + | L | 8.6 | 90.9 | 0.6 |
| pH 6.0 | AMP (5 mM) | | | | |
| 20 mM Citrate, | AMP (5 mM) | M | 15.2 | 84.3 | 0.5 |
| 150 mM NaCl | CaCl2 (2 mM) | N | 15.0 | 84.3 | 0.8 |
| 0.05% PS20, | CaCl2 (2 mM) + | O | 8.6 | 90.9 | 0.5 |
| pH 6.0 | AMP (5 mM) | | | | |

### Example 10: Characterization of Preferred Lyophilized ENPP1 Polypeptide Formulations

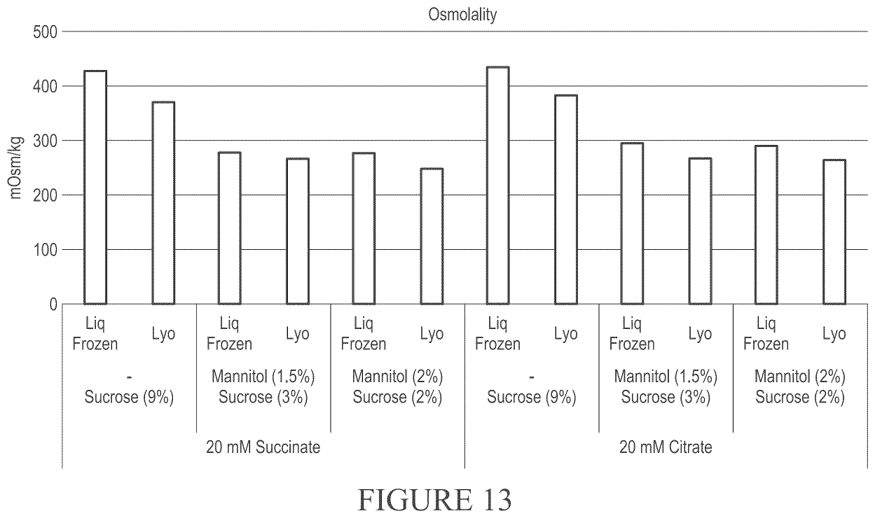

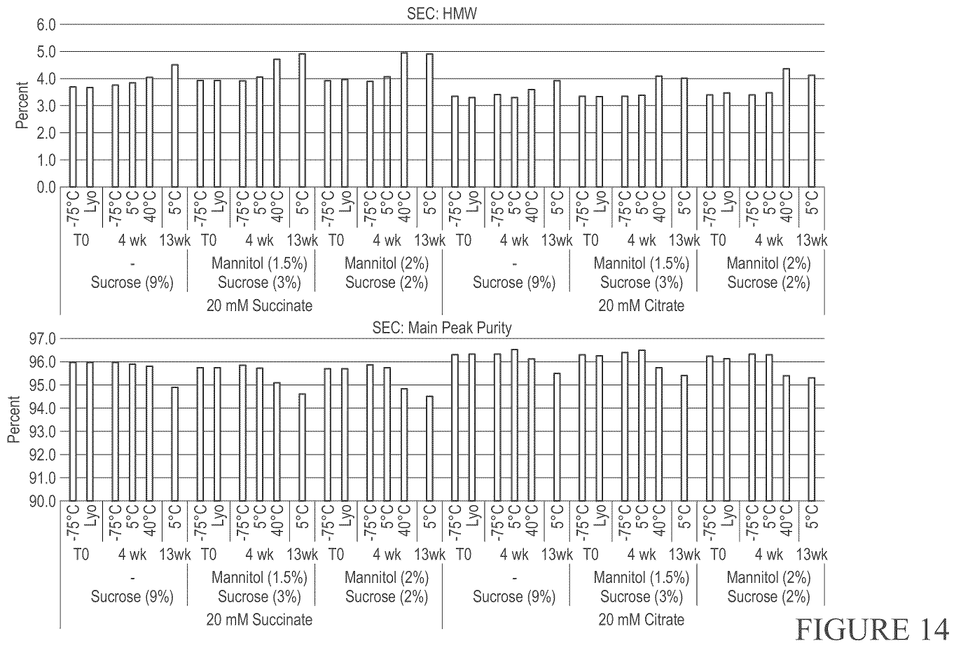

Based on the characterization and stability data presented above, preferred lyophilized ENPP1 polypeptide formulations comprise succinate and citrate. These preferred formulations are enumerated in Table 13, and were further characterized for polypeptide stability over time. The pH was determined to be pH 6.3±0.1 for each formulation sample comprising citrate, and pH 6.1±0.1 for each sample comprising succinate. Osmolality was measured for ENPP1-Fc samples comprising succinate or citrate, and the osmolality values for all samples ranged from 265-432 mOsm/kg. Summary data are shown in FIG. 13. Main peak purity and higher order aggregates of ENPP1-Fc for these formulations was evaluated by SEC. The results are shown in FIG. 14. The lyophilized ENPP1-Fc and controls, in Formulations A, B, C, E and F exhibited relative main peak purity >94%, across all time points and conditions. Succinate samples at the 13 week time point, stored at 5° C. exhibited 1% increase in % HMW content, while citrate samples showed about 0.5% increase in % HMW. Particle size distribution and morphology was evaluated by micro flow imaging analysis (MFI). In MFI, bright-field microscope images are captured in successive frames as a continuous sample stream passes through a flow-cell positioned in the field of view of a microscopic system. Images of the particles present in the sample are processed by image morphology analysis software that allows their quantification in size and count. Representative images and particle concentrations (per mL) were reported for ≥2 μm, ≥5 μm, ≥10 μm, ≥25 μm equivalent circular diameter (ECD) size bins. The ECD of an object is expressed in microns and represents the diameter of a sphere that occupies the same two dimensional surface area as the particle. Results are summarized in Table 14. Total particle counts were generally within acceptable limits for all formulations. Total particle count for the ≥10 μm ECD bin were all below 749 and for the ≥25 μm ECD bin were all below 132, indicating that the formulations disclosed herein lack larger size particulates.

TABLE 14
MFI Summary Data

| | | | | | Total Particle Count per | | | |
|---|---|---|---|---|---|---|---|---|
| | | | Time | | mL | | | |
| Buffer | Excipient | Formulation | Point | Condition | ≥2 | ≥5 | ≥10 | ≥25 |
| 20 mM Succinate, 2 | Sucrose (9%) | A | T0 | −75° C.- | 1505 | 117 | 26 | 7 |
| mM CaCl₂ pH 6.0 | | | | Ctrl | | | | |
| | | | | Lyo | 2902 | 767 | 214 | 12 |
| | | | 4 | −75° C.- | 1835 | 582 | 201 | 34 |
| | | | weeks | Ctrl | | | | |
| | | | | 5° C. | 2301 | 830 | 320 | 35 |
| | | | | 40° C. | 1210 | 398 | 123 | 16 |
| | | | 13 | 5° C. | 4068 | 1223 | 231 | 23 |
| | | | weeks | | | | | |
| | Sucrose (3%) | B | T0 | −75° C.- | 720 | 90 | 17 | 1 |
| | Mannitol (1.5%) | | | Ctrl | | | | |
| | | | | Lyo | 2753 | 763 | 267 | 42 |
| | | | 4 | −75° C.- | 2482 | 800 | 304 | 36 |
| | | | weeks | Ctrl | | | | |
| | | | | 5° C. | 1395 | 446 | 150 | 22 |
| | | | | 40° C. | 839 | 218 | 77 | 8 |
| | | | 13 | 5° C. | 2090 | 652 | 142 | 31 |
| | | | weeks | | | | | |
| | Sucrose (2%) | C | T0 | −75° C.- | 2280 | 659 | 210 | 26 |
| | Mannitol (2%) | | | Ctr | | | | |
| | | | | Lyo | 2634 | 617 | 201 | 23 |
| | | | 4 | −75° C.- | 2953 | 976 | 346 | 65 |
| | | | weeks | Ctr | | | | |
| | | | | 5° C. | 3900 | 976 | 297 | 33 |
| | | | | 40° C. | 877 | 211 | 70 | 15 |
| | | | 13 | 5° C. | 2127 | 615 | 99 | 4 |
| | | | weeks | | | | | |
| 20 mM Citrate, 2 | Sucrose (9%) | D | T0 | −75° C.- | 306 | 81 | 36 | 11 |
| mM CaCl₂ pH 6.0 | | | | Ctrl | | | | |
| | | | | Lyo | 3574 | 386 | 92 | 9 |
| | | | 4 | −75° C.- | 234 | 73 | 31 | 7 |
| | | | weeks | Ctrl | | | | |
| | | | | 5° C. | 818 | 214 | 61 | 10 |
| | | | | 40° C. | 1836 | 754 | 302 | 29 |
| | | | 13 | 5° C. | 4033 | 1951 | 749 | 132 |
| | | | weeks | | | | | |
| | Sucrose (3%) | E | T0 | −75° C.- | 188 | 48 | 17 | 5 |
| | Mannitol (1.5%) | | | Ctrl | | | | |
| | | | | Lyo | 1235 | 195 | 67 | 10 |
| | | | 4 | −75° C.- | 2805 | 1174 | 349 | 34 |
| | | | weeks | Ctrl | | | | |
| | | | | 5° C. | 1389 | 411 | 140 | 22 |
| | | | | 40° C. | 2090 | 850 | 326 | 47 |
| | | | 13 | 5° C. | 1842 | 566 | 121 | 23 |
| | | | weeks | | | | | |
| | Sucrose (2%) | F | T0 | −75° C.- | 718 | 289 | 114 | 26 |
| | Mannitol (2%) | | | Ctrl | | | | |
| | | | | Lyo | 2285 | 357 | 117 | 8 |
| | | | 4 | −75° C.- | 1683 | 639 | 212 | 22 |
| | | | weeks | Ctrl | | | | |
| | | | | 5° C. | 1522 | 406 | 123 | 15 |
| | | | | 40° C. | 1156 | 357 | 107 | 6 |
| | | | 13 | 5° C. | 3182 | 1283 | 492 | 95 |
| | | | weeks | | | | | |

Based on the data reported above, 20 mM citrate, 88 mM sucrose, 82 mM mannitol, 2 mM calcium chloride, 0.05% PS20 pH 6.3 (formulation A) and 20 mM citrate, 263 mM sucrose, 2 mM calcium chloride, 0.05% PS20 pH 6.3 (formulation B) were selected as the most suitable formulations for lyophilized ENPP1-Fc. These formulations were further investigated for their long term effects on lyophilized polypeptide stability at varied storage conditions. The two formulations were tested over a period of three months, at three different storage conditions, 5° C., 25° C., and 40° C. After three months, all lyophilized cakes containing ENPP1-Fc showed intact structures of ivory color. After reconstitution, liquid appearance was observed to be brownish-yellow, clear and free of visible particulates for all sample types. Reconstitution occurred within a window consistent with the data presented above, with full reconstitution of the lyophilized sample occurring within 59 seconds to 108 seconds. The moisture content for both formulations across temperature conditions over time also remained consistent with the moisture content data presented above. All lyophilized sample time points at all of the tested storage temperatures possessed very low or no moisture content across time points and conditions, and residual moisture content ranged from 0.0%-0.3%. pH values for each sample at each storage condition also remained consistent over time, with sample pH remaining at pH 6.3±0.1 over the course of three months. Protein recovery relative to the 50 mg/mL normalized starting concentrations was also measured for each sample. Summary protein recovery data are presented in Table 15. Recovery of each sample was consistent with recovery data for the zero time point positive control. The zero time point positive control was measured to have a concentration of 40.1 mg/mL. All recovered samples presented concentrations either within 0.3 mg/mL below the positive control sample, or within 8.8 mg/mL above the concentration of the positive control sample. These data indicate that protein recovery does not degrade over time under 5° C., 25° C., and 40° C. storage conditions.

TABLE 15
Summary Preferred Formulation Protein Recovery Data Over Three
Months at 5° C. 25° C., and 40° C.

| | | Recon- | | | |
|---|---|---|---|---|---|
| | Nominal | stitution | | | Recovery |
| | Conc. | Fill Vol | Time | | by A280 |
| Sample | (mg/mL) | (mL) | Point | Condition | (mg/mL) |
| Formulation | 50.0 | 0.5 | T 0 | Frozen Ctrl | 47.5 |
| A | | | | Lyo | 40.1 |
| | | | 1 Month | Frozen Ctrl | 43.4 |

| | | |
|---|---|---|
| 5° | C. | 43.1 |
| 25° | C. | 40.2 |
| 40° | C. | 39.8 |

| | | |
|---|---|---|
| 3 Month | Frozen Ctrl | 46.8 |

| | | |
|---|---|---|
| 5° | C. | 43.9 |
| 25° | C. | 40.7 |
| 40° | C. | 46.3 |

| | | | |
|---|---|---|---|
| Formulation | T 0 | Frozen Ctrl | 53.9 |
| B | | Lyo | 48.5 |
| | 1 Month | Frozen Ctrl | 52.7 |

| | | |
|---|---|---|
| 5° | C. | 48.0 |
| 25° | C. | 47.1 |
| 40° | C. | 48.9 |

| | | |
|---|---|---|
| 3 Month | Frozen Ctrl | 55.3 |

| | | |
|---|---|---|
| 5° | C. | 49.4 |
| 25° | C. | 48.9 |
| 40° | C. | 49.9 |

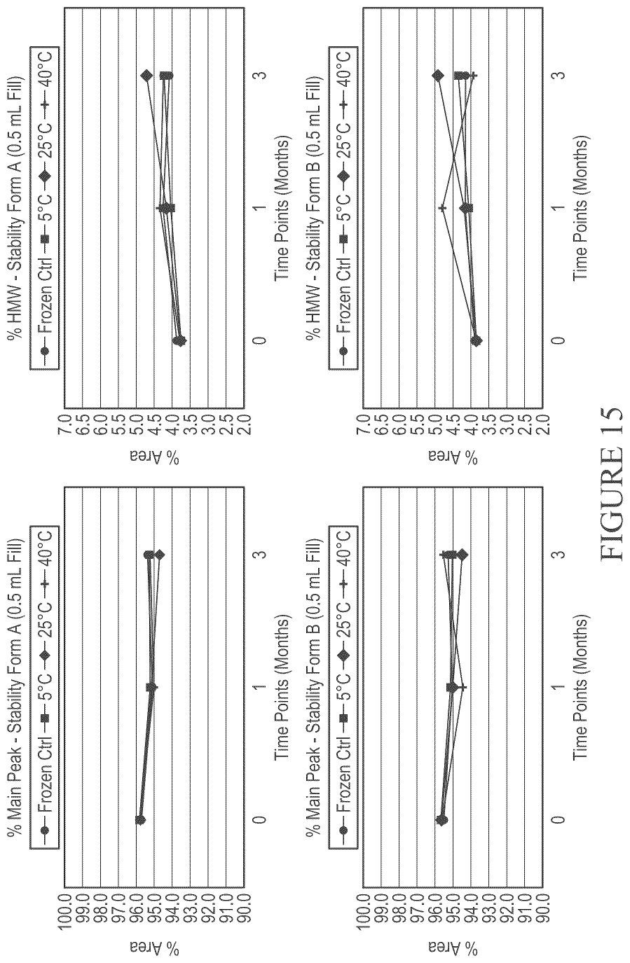

SEC experiments were performed to characterize the propensity for HMW species to develop over a period of three months upon storage of samples of preferred formulations were at 5° C., 25° C., and 40° C. Summary SEC data pertaining to percent abundances for total HMW species, LMW species, and main peak species are reported in Table 16. FIG. 15 shows summary SEC data for percent abundance HMW and percent abundance main peak over time, per storage condition. A positive control zero time point sample possessed approximately 4% HMW content. All preferred formulation samples remained consistent with the control, each having ±5% HMW content over a period of three months under 5° C., 25° C., and 40° C. storage conditions. These results indicate that each preferred formulation is surprisingly capable of preventing the formation of high molecular weight aggregate species for extended amounts of time of at least three months. These data also indicate that the preferred formulations are capable of stabilizing the lyophilized polypeptide against formation of HMW species even when stored at higher or non-refrigerated temperatures such as 25° C. and 40° C.

TABLE 16
Summary SEC Data for Preferred Formulations Over Three Months at 5° C., 25° C., and 40° C.

| | | | | | | | Main | |
|---|---|---|---|---|---|---|---|---|
| | Nominal | | | | | | Peak | |
| | Conc. | Reconstitution | Sample | | | HMW | Purity | LMW |
| Sample | (mg/mL) | Fill Vol (mL) | Type | Time Point | Condition | (%) | (%) | (%) |
| Formulation | 50.0 | 0.5 | Lyo | T0 | Frozen Ctrl | 3.8 | 95.7 | 0.5 |
| A | | | | | Lyo | 3.7 | 95.8 | 0.5 |
| | | | | 1 Month | Frozen Ctrl | 4.2 | 95.1 | 0.7 |
| | | | | | 5° C. | 4.0 | 95.2 | 0.7 |
| | | | | | 25° C. | 4.1 | 95.1 | 0.8 |
| | | | | | 40° C. | 4.3 | 95.0 | 0.7 |
| | | | | 3 Month | Frozen Ctrl | 4.0 | 95.3 | 0.6 |
| | | | | | 5° C. | 4.2 | 95.2 | 0.6 |
| | | | | | 25° C. | 4.7 | 94.7 | 0.6 |
| | | | | | 40° C. | 4.2 | 95.2 | 0.6 |
| Formulation | | | | T0 | Frozen Ctrl | 3.9 | 95.5 | 0.6 |
| B | | | | | Lyo | 3.9 | 95.6 | 0.5 |
| | | | | 1 Month | Frozen Ctrl | 4.2 | 95.1 | 0.8 |
| | | | | | 5° C. | 4.1 | 95.1 | 0.8 |
| | | | | | 25° C. | 4.2 | 95.1 | 0.8 |
| | | | | | 40° C. | 4.8 | 94.5 | 0.8 |
| | | | | 3 Month | Frozen Ctrl | 4.1 | 95.3 | 0.6 |
| | | | | | 5° C. | 4.3 | 95.1 | 0.6 |
| | | | | | 25° C. | 4.9 | 94.5 | 0.6 |
| | | | | | 40° C. | 4.0 | 95.5 | 0.6 |

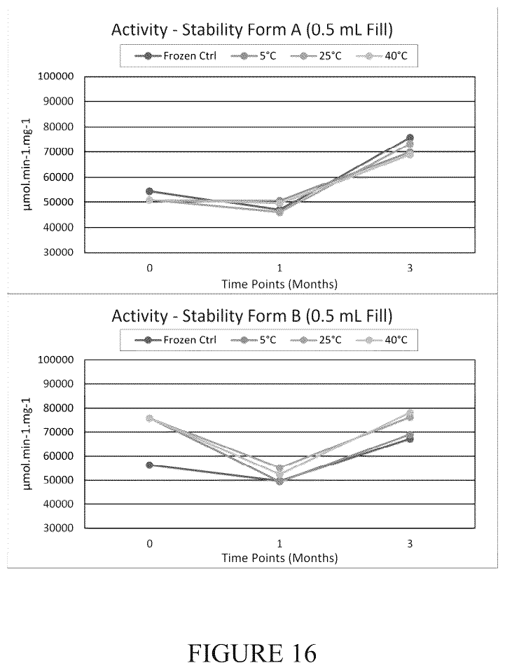

The specific activity for all preferred formulation samples corresponding to each time point and storage condition was assessed utilizing the enzymatic activity screen described above. Summary data is shown in Table 17. The activity data was plotted over time for each sample including a frozen control. Representative activity vs time plots are shown in FIG. 16. All samples across time points and storage conditions presented consistent levels of enzymatic activity with the positive control, with activity levels ranging from 46009 μmol min⁻¹ mg⁻¹ to 73173 μmol min⁻¹ mg⁻¹.

TABLE 17
Summary Enzymatic Activity Data for Preferred Formulations
Over Three Months at 5° C., 25° C., and 40° C.

| | | | | | | Specific | |
|---|---|---|---|---|---|---|---|
| | | | | | | Activity | |
| | Nominal | Fill | Sam- | | | (μmol · | |
| | Conc. | Vol | ple | Time | | min⁻¹ · | Std. |
| Sample | (mg/mL) | (mL) | Type | Point | Condition | mg⁻¹) | Dev. |
| For- | 50.0 | 0.5 | Lyo | T0 | Frozen Ctrl | 54374 | 2181 |
| mulation A | | | | | Lyo | 50840 | 3443 |
| | | | | 1 Month | Frozen Ctrl | 46899 | 1679 |
| | | | | | 5° C. | 50717 | 2480 |
| | | | | | 25° C. | 46009 | 1776 |
| | | | | | 40° C. | 49521 | 2633 |
| | | | | 3 Month | Frozen Ctrl | 75625 | 344 |
| | | | | | 5° C. | 69830 | 1623 |
| | | | | | 25° C. | 73173 | 1233 |
| | | | | | 40° C. | 68859 | 611 |
| For- | | | | T0 | Frozen Ctrl | 56227 | 3859 |
| mulation B | | | | | Lyo | 75734 | 911 |
| | | | | 1 Month | Frozen Ctrl | 49683 | 481 |
| | | | | | 5° C. | 49343 | 652 |
| | | | | | 25° C. | 55132 | 3391 |
| | | | | | 40° C. | 52421 | 1210 |
| | | | | 3 Month | Frozen Ctrl | 67093 | 385 |
| | | | | | 5° C. | 68920 | 2554 |
| | | | | | 25° C. | 76087 | 803 |
| | | | | | 40° C. | 78107 | 2883 |

Particle size distribution and morphology for all preferred formulation time point samples was evaluated by MFI analysis. Representative images and particle concentrations (per mL) were reported for ≥2 μm, ≥5 μm, ≥10 μm, ≥25 μm ECD size bins. Results are summarized in Table 18. Total particle counts were surprisingly well below those observed in the MFI results described above, and were within acceptable limits for all preferred formulation samples. Total particle count for the ≥10 μm ECD bin were all below 146, and for the ≥25 μm ECD bin were all below 21, indicating that the preferred formulations disclosed herein are capable of a nearly tenfold reduction in larger size particulates when compared to other formulation combinations described in the previous MFI analysis.

TABLE 18
Summary MFI Data for Preferred Formulations Over
Three Months at 5° C., 25° C., and 40° C.

| | No- | | | | | | | | |
|---|---|---|---|---|---|---|---|---|---|
| | minal | | | | | | | | |
| | Conc. | Fill | Sam- | | | Total Particle | | | |
| Sam- | (mg/ | Vol | ple | Time | Con- | Count/mL | | | |
| ple | mL) | (mL) | Type | Point | dition | ≥2 | ≥5 | ≥10 | ≥25 |
| For- | 50.0 | 0.5 | Lyo | T0 | Frozen | 2128 | 504 | 103 | 14 |
| mula- | | | | | Ctrl | | | | |
| tion | | | | | Lyo | 27676 | 2200 | 20 | 8 |
| A | | | | 1 | Frozen | 1473 | 523 | 146 | 10 |
| | | | | Month | Ctrl | | | | |
| | | | | | 5° C. | 2033 | 675 | 101 | 10 |
| | | | | | 25° C. | 1856 | 571 | 80 | 12 |
| | | | | | 40° C. | 2037 | 735 | 142 | 21 |
| | | | | 3 | Frozen | 2760 | 410 | 93 | 10 |
| | | | | Month | Ctrl | | | | |
| | | | | | 5° C. | 521 | 76 | 14 | 0 |
| | | | | | 25° C. | 412 | 70 | 21 | 0 |
| | | | | | 40° C. | 424 | 45 | 0 | 0 |
| For- | | | | T0 | Frozen | 591 | 103 | 37 | 0 |
| mula- | | | | | Ctrl | | | | |
| tion | | | | | Lyo | 28553 | 1961 | 43 | 6 |
| B | | | | 1 | Frozen | 1134 | 412 | 86 | 8 |
| | | | | Month | Ctrl | | | | |
| | | | | | 5° C. | 2393 | 354 | 35 | 2 |
| | | | | | 25° C. | 3124 | 379 | 21 | 0 |
| | | | | | 40° C. | 1864 | 360 | 39 | 0 |
| | | | | 3 | Frozen | 1114 | 140 | 29 | 0 |
| | | | | Month | Ctrl | | | | |
| | | | | | 5° C. | 1988 | 64 | 12 | 2 |
| | | | | | 25° C. | 564 | 27 | 6 | 0 |
| | | | | | 40° C. | 342 | 76 | 19 | 2 |

The above formulation development studies focused on identification of formulation components that would result in optimal chemical, physical and structural stability of ENPP1-Fc formulations under both stressed and non-stressed conditions. Multiple buffer types, pH conditions, excipients, and surfactant concentrations were evaluated in an iterative fashion over the course of baseline buffer, excipient, solubility, surfactant, and cofactor studies. Based on the results for the lyophilized formulation studies disclosed herein, 20 mM citrate pH 6.3, 88 mM sucrose, 82 mM mannitol, 2 mM calcium chloride, 0.05% PS20 and 20 mM citrate pH 6.3, 263 mM sucrose, 2 mM calcium chloride, 0.05% PS20 were selected as the most suitable formulations for ENPP1-Fc. These preferred formulations confer maximal chemical, physical, and conformational stability to ENPP1-Fc at a target concentration of 50 mg/mL lyophilized polypeptide formulations.

In addition, another exemplary formulation was tested (Formulation C1): 50 mg/mL of an ENPP1 polypeptide, 20 mM citrate at about pH 6.3, 2 mM calcium chloride, 175 mM sucrose, 82 mM (D) mannitol, and 0.05% w/v polysorbate 20. This formulation was investigated for its long-term effects on lyophilized ENPP1-Fc polypeptide stability at varied storage conditions over 12 months at three different storage conditions: 5° C., 25° C., and 40° C. After three-month increments, lyophilized cakes containing ENPP1-Fc showed intact structures of ivory color. After reconstitution, liquid appearance was observed to be brownish-yellow, clear and free of visible particulates for all sample types. Reconstitution occurred within a window consistent with the data presented above, with full reconstitution of the lyophilized sample occurring within two to three minutes. The moisture content across temperature conditions over time also remained consistent. All lyophilized sample time points at all of the tested storage temperatures possessed very low or no moisture content across time points and conditions, and residual moisture content ranged from 0.0%-0.16%. pH values for each sample at each storage condition also remained consistent over time, with sample pH remaining at pH 6.3±0.1 over the course of three months.

Size exclusion chromatography (SEC) was used to evaluate the quantity of aggregates and degradation products present in ENPP1 polypeptide formulation samples. Percent abundances for total high molecular weight (HMW) species, low molecular weight (LMW) species, and main peak purity over a twelve (12) month (5° C. and 25° C.) and six month (40° C.) period.

TABLE 19
Summary Enzymatic Activity Data for Formulation C1 Over
Twelve Months at 5° C.

| Ana- | | | | | | | |
|---|---|---|---|---|---|---|---|
| lytical | | | | | | | |
| Pro- | | Time | One | Three | Six | Nine | Twelve |
| cedure | Units | Zero | Month | Months | Months | Months | Months |
| Specific | U/ | 90.10 | 98.30 | 96.40 | 81.80 | 99.90 | 90.90 |
| Activity | mg- | | | | | | |
| | umol/ | | | | | | |
| | min* | | | | | | |
| | mg | | | | | | |
| SE- | Area | 3.8 | 4.1 | 4.0 | 4.0 | 3.8 | 3.6 |
| HPLC | % | | | | | | |
| HMWS | | | | | | | |
| SE- | Area | <0.3 | <0.3 | <0.3 | <0.3 | <0.3 | <0.3 |
| HPLC | % | | | | | | |
| LMWS | | | | | | | |
| SE- | Area | 96.1 | 95.8 | 95.8 | 95.9 | 96.1 | 96.3 |
| HPLC | % | | | | | | |
| Main | | | | | | | |
| Peak | | | | | | | |

TABLE 20
Summary Enzymatic Activity Data for Formulation
C1 Over Twelve Months at 25° C.

| Ana- | | | | | | | |
|---|---|---|---|---|---|---|---|
| lytical | | | | | | | |
| Pro- | | Time | One | Three | Six | Nine | Twelve |
| cedure | Units | Zero | Month | Months | Months | Months | Months |
| Specific | U/ | 90.10 | 101.80 | 91.20 | 86.70 | 101.40 | 92.70 |
| Activity | mg- | | | | | | |
| | umol/ | | | | | | |
| | min* | | | | | | |
| | mg | | | | | | |
| SE- | Area | 3.8 | 4.3 | 4.4 | 4.4 | 4.2 | 3.9 |
| HPLC | % | | | | | | |
| HMWS | | | | | | | |
| SE- | Area | <0.3 | <0.3 | <0.3 | <0.3 | <0.3 | <0.3 |
| HPLC | % | | | | | | |
| LMWS | | | | | | | |
| SE- | Area | 96.1 | 95.6 | 95.4 | 95.4 | 95.7 | 96.0 |
| HPLC | % | | | | | | |
| Main | | | | | | | |
| Peak | | | | | | | |

TABLE 21
Summary Enzymatic Activity Data for Formulation
C1 Over Six Months at 40° C.

| Analytical | | Time | One | Three | Six |
|---|---|---|---|---|---|
| Procedure | Units | Zero | Month | Months | Months |
| Specific | U/mg − | 90.10 | 102.90 | 96.0 | 85.90 |
| Activity | umol/min*mg | | | | |
| SE-HPLC | Area % | 3.8 | 4.5 | 4.9 | 5.0 |
| HMWS | | | | | |
| SE-HPLC | Area % | <0.3 | <0.3 | <0.3 | <0.3 |
| LMWS | | | | | |
| SE-HPLC | Area % | 96.1 | 95.3 | 94.9 | 94.8 |
| Main Peak | | | | | |

The specific activity and SEC data demonstrate that Formulation C1, like formulations A and B (above), provides marked overall stability for ENPP1-Fc, here particularly over a range of temperatures and for at least twelve months.

### Example 11: Treatment Using Lyophilized ENPP1 Polypeptide Formulations

ENPP1-Fc can be administered with any of the aforesaid formulations disclosed. In some embodiments, ENPP1-Fc is administered in a formulation comprising: 20 mM citrate at about pH 6.3, 2 mM calcium chloride, 175 mM sucrose, 82 mM (D) mannitol, and 0.05% w/v polysorbate 20. A lyophilized formulation is reconstituted in a suitable reconstitution buffer (or sterile water) prior to administration to a subject in need thereof.

ENPP1-Fc is administered at one of the following selected doses: 0.2 mg/kg, 0.6 mg/kg, and 1.8 mg/kg. Administration is subcutaneous (SC) at least once or twice bimonthly, at least once or twice monthly, three times monthly, at least once or twice weekly.

The first dose of ENPP1-Fc may be administered on Day 1. On Days 8 and thereafter, ENPP1-Fc is administered to a subject at a selected dose of ENPP1 twice weekly. The dose may be administered at approximately the same time on each dosing day. The site of injection is alternated, with no site within 2 inches of any prior site of injection within the prior 2 weeks.

A selected dose of ENPP1-Fc is one of 0.2 mg/kg, 0.6 mg/kg, or 1.8 mg/kg SC. The first dose of ENPP1-Fc may be administered on Day 1. After the first dose, a subject may be observed for 7 days to monitor safety and to collect PK samples. On Days 8 and thereafter, a subject receives a selected dose twice weekly. Administration of ENPP1-Fc at a selected dose is continued as considered appropriate by the medical professional.

A subject may receive 8 doses of ENPP1-Fc over the course of a 29 day period of time, for example, resulting in an exposure of 1.6 mg, 4.8 mg, and 14.4 mg per 29 days, respectively, for dose amounts of 0.2 mg/kg, 0.6 mg/kg, and 1.8 mg/kg. Or a subject may receive more or less than 8 doses, as considered appropriate by a medical profession.

Like the endogenous ENPP1 enzyme, ENPP1-Fc cleaves ATP to generate AMP and PPi, thereby increasing plasma PPi levels and into AMP which CD73 coverts rapidly to adenosine. Replacement of the endogenous human enzyme is intended to correct the inherent deficiency and allow for improved health and mitigation of clinical complications associated with ENPP1 Baseline patient, clinician, and caregiver outcomes.

### Example 12: Treatment of a Patient Having an ENPP1 Deficiency

Enpp1-Fc formulated as above is administered to a patient identified as having an ENPP1 deficiency by subcutaneous injection on Day 1 and twice weekly starting on Day 8 using a select dose as follows.

TABLE 22
Exemplary Dosages

| | |
|---|---|
| 1 | 0.2 mg/kg |
| 2 | 0.6 mg/kg |
| 3 | 1.8 mg/kg |

ENPP1-Fc is administered at a selected dose of ENPP1-FC is one of 0.2 mg/kg, 0.6 mg/kg, or 1.8 mg/kg SC at least twice weekly for a period of time determined by the medical professional. The Patient's response to enzyme replacement is monitored as appropriate, as determined by the medical professional, e.g., by following a reduction in one or more symptoms of ENPP1 deficiency, and/or using guidance provided herein.

### Example 13: Treatment of a Patient Diagnosed with GACI

Enpp1 deficiency may mask as GACI. GACI is a rare disease occurring in infants and involving extensive arterial calcification (Albright, et al., 2015, Nature Comm. 10006).

ENPP1-Fc formulated as above is administered at a selected dose of ENPP1-FC is one of 0.2 mg/kg, 0.6 mg/kg, or 1.8 mg/kg SC at least twice weekly for a period of time determined by the medical professional. GACI Patient response to enzyme replacement is monitored as appropriate, as determined by the medical professional, e.g., by following a reduction in one or more symptoms of GACI, and/or using guidance provided herein.

### Example 14: Treatment of Patient Diagnosed with ARHR2

Enpp1 deficiency may mask as ARHR2. ARHR2 is a rare skeletal disorder characterized by low levels of plasma PPi and serum phosphate which can result in rickets, repeated fractures of the long bones, rachitic skeletal deformities and impaired growth and development (Ferreira et al 2014, Moran 1975, Rutsch et al 2008).

ENPP1-Fc formulated as above is administered at a selected dose of ENPP1-Fc is one of 0.2 mg/kg, 0.6 mg/kg, or 1.8 mg/kg SC at least twice weekly for a period of time determined by the medical professional. ARHR2 Patient response to enzyme replacement is monitored as appropriate, as determined by the medical professional, e.g., by following a reduction in one or more symptoms of ARHR2, using guidance provided herein.

The following examples, in the context of the entire specification, provide guidance to determine treatment protocols and efficacy.

### Example 15: Measurement of Plasma Inorganic Pyrophosphate

Low plasma PPi levels are a characteristic of ENPP1 Deficiency and are used as an indicator of treatment efficacy. ENPP1-Fc specifically cleaves ATP to generate AMP and PPi. The therapeutic goal of ENPP1 ERT is to normalize extracellular PPi levels and correct clinical abnormalities associated with ENPP1 Deficiency.

PPi is measured by obtaining patient plasma samples. Determined PPi data may be used to adjust dose levels. PPi levels may also serve as the primary PD marker for PK/PD analysis.

The concentration of Pi and PPi in mammals is 1-3 mM and 2-3 pM respectively.

### Example 16: Biomarkers Associated with Bone Health

In addition to low plasma PPi, ENPP1 deficient patients are characterized biochemically by low serum phosphate, high urine phosphate, low renal TmP/GFR, normal calcium (Ca), low-normal urine Ca, normal 25-hydroxy Vitamin D (25 OH D), low-normal 1,25 (OH) 2D, high BAP, high intact FGF23, and normal PTH (IOF 2019).

Biomarkers that may be used as additional determinants of bone health of a treated patient are set forth in Table 19.

TABLE 23
Clinical Intermediates and Biomarkers

| | Laboratory | Sample Type |
|---|---|---|
| Primary | Pyrophosphate (PPi) | plasma |
| Pharmacodynamic | Inorganic phosphate | serum |
| Markers | FGF23 (intact) | Plasma |
| | TmP/GFR | serum creatinine, serum |
| | | phosphate, urine |
| | | phosphate |
| | ALP, BALP, CTx, P1NP | serum, plasma, urine |

### Example 17: Efficacy of Treatment with ENPP1-Fc

Treatment efficacy may be assessed by measuring plasma PPi as well as measuring other plasma analytes, such as FGF23, Pi, FGF23, Pi, TmP/GFR, serum alkaline phosphatase (ALP), bone-specific ALP (BALP), carboxy terminal cross-linked telopeptide of type I collagen (CTx), and procollagen type 1 N-terminal propeptide (P1NP). These analyte measurements may be used as a PD markers associated with ENPP1 Deficiency to determine the efficacy for ENPP1-FC. Changes in these analytes may be described as changes from baseline and in a time-dependent manner over the course of treatment. Dose linearity of PK and PD parameters also may be assessed.

Changes from baseline in plasma PPi levels, FGF23 levels and Urinary phosphorus excretion per creatinine clearance may be analyzed using a t test of paired differences to test the null hypothesis that the change from baseline of PPi levels is equal to zero.

### Example 18: Drug Concentration Measurements

In addition, blood samples may be obtained from a patient for measurement of ENPP1-FC concentration in plasma and subsequent determination of PK parameters following the first dose (i.e. single dose) and at/after multiple doses (i.e., steady-state).

### Example 19: Immunogenicity (Anti-Drug Antibodies)

If desired, immunogenicity to ENPP1-Fc may be measured using anti-drug antibodies (ADA). Immunogenicity testing can utilize a multi-tiered approach; if ADA are detected in the initial screen, a confirmatory test may be run to determine specificity. Samples may also be used to assess and further establish assays for specificity confirmation (i.e., titer) and neutralizing antibodies.

### Example 20: Pharmacokinetic, Pharmacodynamic, and Exploratory Biomarker Analyses

Pharmacokinetic analysis may be performed on the PK population, and PK parameters of ENPP1-FC may be summarized by treatment with descriptive statistics. Dose linearity of PK and PD parameters may also be assessed. PK/PD analyses, immunogenicity analyses; and exploratory biomarker analyses may be determined.

### Example 21: Additional Determinators of Efficacy

Although restoring a normal level of PPi is the primary indicator of efficacy of treatment using NEPP1-Fc, other physical measurements also may be used, if desired to assist in determining treatment efficacy. These include one or more of the following.

### 1. Radiography and Imaging

X-Rays for Skeletal Severity. Standard X-rays may be obtained to detect rachitic skeletal deformities. Obtain X-rays may be obtained, for example, on the wrists and knees.

DEXA Scan. DEXA scans may be used to evaluate changes in bone density.

Positron Emission Tomography-Computed Tomography. Baseline Na¹⁸F-PET/HRpQCT (or HR-CT) may be a full body scan done within 1 month of first dose of ENPP1-FC to measure calcification of arteries and organs and skeletal abnormalities at baseline and for future interventional assessments. The Na¹⁸F-PET measures bone turnover as well as microcalcification of the arteries. High-resolution quantitative computed tomography (HRQCT) or HR-CT can determine bone microstructure at the non-dominant distal radius and tibia. Standard bone geometric parameters are calculated.

Doppler Echocardiogram. A baseline echocardiogram may be obtained within 3 days prior to a first dose of ENPP1-FC. Doppler echo may be used to measure heart function [LVEF, blood flow] calcification of heart and valves, and arterial stiffness.

Optical Coherence Tomography. Optical coherence tomography may be used to visualize neointimal proliferation.

Peripheral Arterial Tonometry. Peripheral arterial tonometry (PAT) may be used to assess digital pulse wave amplitude (PWA), which corresponds to digital volume variation.

Renal Ultrasound. Renal ultrasound may be used, for example, within 1 week of starting ENPP1-FC, to measure renal calcification.

Bone Histomorphology and Bone Biopsy. Bone biopsy may be performed as a baseline measurement. Tetracycline loading for 10 days prior to bone biopsy is preferred.

2. Walking Ability. Walk tests may be used as a submaximal exercise measurement to measure functional capacity in ambulatory patients combining cardiopulmonary, neuromuscular, and musculoskeletal functions. The 6 Minute Walk Test (6MWT) was originally developed by the American Thoracic Society (ATS 2002) for use with adults, and is now commonly used in both adult and pediatric populations (Mylius et al 2016), and with children with neuromuscular diseases such as spinal muscular atrophy (Montes et al 2018), Duchenne muscular dystrophy (McDonald et al 2013), and infantile-onset Pompe disease (van der Meijden et al 2018). The 2 Minute Walk Test (2MWT) is included in the NIH Toolbox and is increasingly being used to measure the same properties.

The 6MWT and the 2MWT may be administered to the patient before and during treatment at the discretion of the healthcare provider. If a subject is unable to complete at least the 2MWT at baseline, additional assessments during treatment may be left to a healthcare provider's discretion. Resting heart rate is obtained prior to the test and post-test. Distance walked in the first 2 minutes of the 6MWT and the full 6 minutes may be recorded. The distances walked in 2 minutes and 6 minutes may be compared to age- and sex-matched normative data (percent predicted values).

3. Dynamometry. Strength may be assessed using dynamometry before and/or during treatment at the discretion of the healthcare provider. Hand-held dynamometry is a direct measurement of strength commonly used in both children and adults. Muscle groups that may be assessed include: shoulder abduction, shoulder flexion, elbow flexion, elbow extension, hip abduction, hip flexion, hip extension, and knee extension. Each muscle group may be measured 2 times bilaterally.

Grip Strength. Grip strength may be measured using a grip strength dynamometer before and/or during treatment at the discretion of the healthcare provider. Equipment and assessor instructions may be standardized across sites. Grip may be assessed bilaterally with 1 practice and 1 maximal force measures taken for each hand and results may be compared to age and gender matched normative data (when available).

Range of Motion. Range of Motion may be assessed using a goniometer, an instrument that tests the angle of joints and measures the degree of movement at a joint. The stationary arm of the goniometer is aligned with the specified bony landmark on the stationary body segment, and the moving arm of the goniometer is aligned with the specified bony landmark of the limb that is moving. The fulcrum of the goniometer is specified for each motion measured using axis of motion and bony landmarks. Range of motion may be assessed for one or more of the following: shoulder abduction, shoulder flexion, elbow flexion, elbow extension, hip abduction, hip flexion, hip extension, and knee extension.

4. Hearing Testing. Moderate hearing loss has been associated with ARHR2 (Brachet et al 2014, Steichen-Gersdorf et al 2015). Baseline hearing may be determined by one or more of: Physical exam and otoscopy, Immittance audiometry (commonly called tympanometry), Pure Tone Audiometry (PTA) with frequencies up to 8 kHz if possible. (If there is a PTA threshold of >15 dB, the subject should also undergo bone conduction testing), High Frequency Audiometry (HFA), with frequencies up to 16 kHz.

5. Clinician Global Impression Scales. The Clinical Global Impression (CGI-S) scales were developed for use in National Institute of Mental Health-sponsored clinical studies to provide a brief, stand-alone assessment of the clinician's view of the patient's global functioning prior to and after initiating a study medication (Guy 1976). The CGI provides an overall clinician-determined summary measure that considers all available information, including knowledge of the patient's history, psychosocial circumstances, symptoms, behavior, and the impact of the symptoms on the patient's ability to function. The CGI-S may be administered before and/or during treatment at the discretion of the healthcare provider and provides a global assessment of change using a seven-point scale ranging from −3 (severe worsening) to +3 (significant improvement).

6. Gross Motor Function Classification System-Expanded and Revised. The Gross Motor Classification System-Expanded and Revised (GMFCS-E and R) may be administered before and/or during treatment at the discretion of the healthcare provider. The GMFCS-E and R classifies patient-initiated movement with an emphasis on mobility on a scale from 1 to 5.

7. Patient Reported Outcomes Measurement Information Systems. The Patient Reported Outcomes Measurement Information Systems (PROMIS) consists of a variety of questionnaires developed by the National Institutes of Health (NIH) to evaluate physical, mental, and social well-being from the patient perspective (http://www.healthmeasures.net). These questionnaires have been used in clinical studies in people with chronic health conditions such as X-linked hypophosphatemia, arthritis, multiple sclerosis, and neurofibromatosis. Each questionnaire contains 8 to 10 items which are rated by the participant on a 5-point Likert scale ranging from 1 (never) to 5 (always). Scores are summed for each questionnaire, with high scores indicating more of the domain being measured (e.g., more fatigue, more physical function). Raw scores are converted to T-Scores based on a mean of 50 and a standard deviation of 10, allowing comparison of the study sample to the general population. PROMIS Scales may include the Pain Interference (short form 8a), Pain Intensity (version 3a), Physical Function—Upper Extremity (custom short form), Physical Function-Mobility (short form 13a FACIT Fatigue), Fatigue (short form), and Cognitive Impact (short form 8a) and may be administered before and/or during treatment at the discretion of the healthcare provider. These assessments may be completed by the subject without assistance.

8. Caregiver Global Impression Scales. The Caregiver Global Impression of Status may be administered to the patient's caregiver before and/or during treatment at the discretion of the healthcare provider. The Caregiver Global Impression of Change provides a global assessment of change using a seven-point scale ranging from −3 (severe worsening) to +3 (significant improvement).

9. Western Ontario and McMaster University Osteoarthritis Index. The WOMAC is a patient-reported outcome used to assess activities of daily living, functional mobility, gait, general health, pain, and quality of life in patients with hip or knee pain (www.sralab.org). The assessment consists of 24-items and takes approximately 12 minutes to administer. The WOMAC may be administered before and/or during treatment at the discretion of the healthcare provider. The assessment may be completed by the subject without assistance.

### Example 22: Extended Stability Study of Lyophilized ENPP1 Polypeptide Formulations

The lyophilized ENPP1-Fc in preferred formulations were tested for long term stability when stored over a period of 36 months, at three different storage conditions, 5° C., 25° C., and 40° C. After 24 months, all lyophilized cakes containing ENPP1-Fc showed intact structures of ivory color. After reconstitution in water, liquid appearance was observed to be brownish-yellow, clear and free of visible particulates for all sample types. Reconstitution occurred within a window consistent with the data presented above, with full reconstitution of the lyophilized sample occurring within 59 seconds to 108 seconds.

The moisture content for both formulations across temperature conditions over time also remained consistent with the moisture content data presented above. All lyophilized sample time points (0, 3, 6, 9, 12, 18, 24, 30, 36 months) at all of the tested storage temperatures possessed very low or no moisture content across time points and conditions, and residual moisture content ranged from 0.0%-0.3%. pH values for each sample at each storage condition also remained consistent over time, with sample pH remaining at pH 6.3±0.1 over the course of 36 months. Protein recovery relative to the 50 mg/mL normalized starting concentrations was also measured for each sample. These data indicate that protein recovery does not degrade over time under 5° C., 25° C., and 40° C. storage conditions.

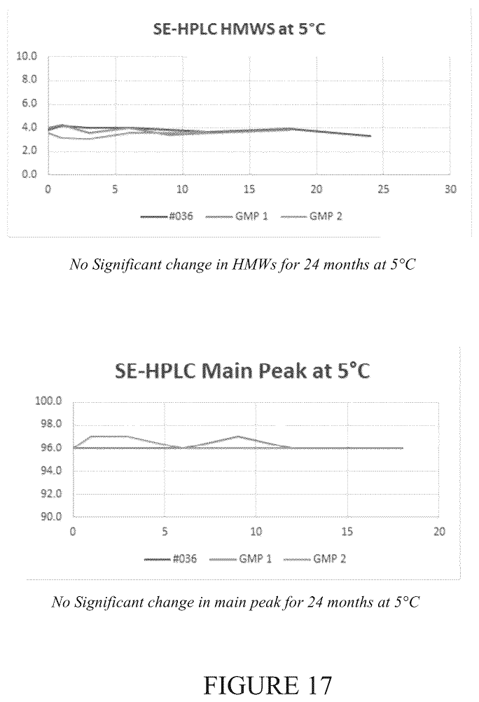

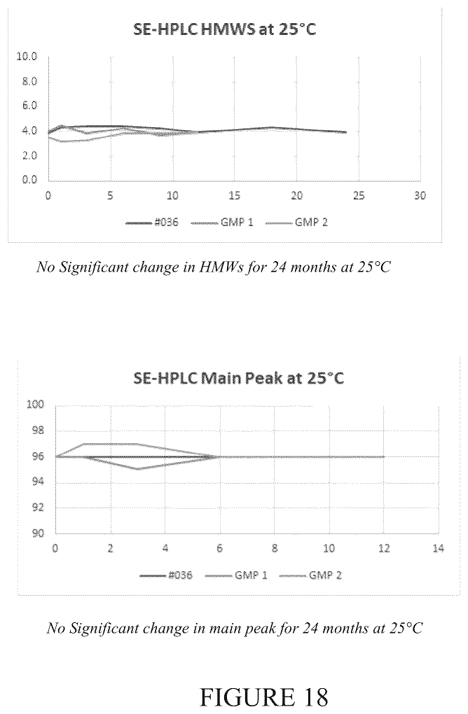

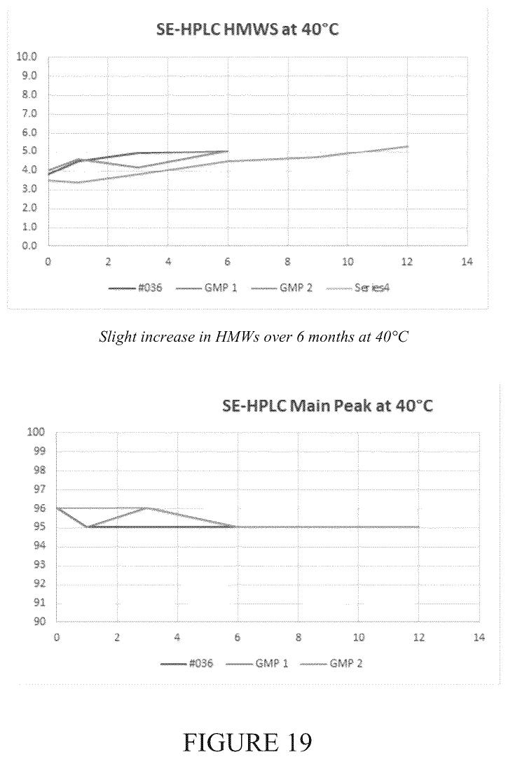

SEC experiments were performed to characterize the propensity for HMW species to develop over a period of 36 months upon storage of samples of preferred formulations were at 5° C., 25° C., and 40° C. Summary SEC data pertaining to percent abundances for total HMW species, LMW species, and main peak species were collected following the procedures outlined in prior examples. FIGS. 17-19 shows summary SEC data for percent abundance HMW and percent abundance main peak over time for 5° C., 25° C. and 40° C. respectively. A positive control zero time point sample possessed approximately 4% HMW content. All preferred formulation samples remained consistent with the control, each having ±5% HMW content over a period of at least 24 months under 5° C., 25° C., and 40° C. storage conditions. These results indicate that each preferred formulation is surprisingly capable of preventing the formation of high molecular weight aggregate species for extended amounts of time. Similar results were seen for samples stored up to 36 months. These data also indicate that the preferred formulations are capable of stabilizing the lyophilized polypeptide against the formation of HMW species even when stored at higher or non-refrigerated temperatures such as 25° C. and 40° C.

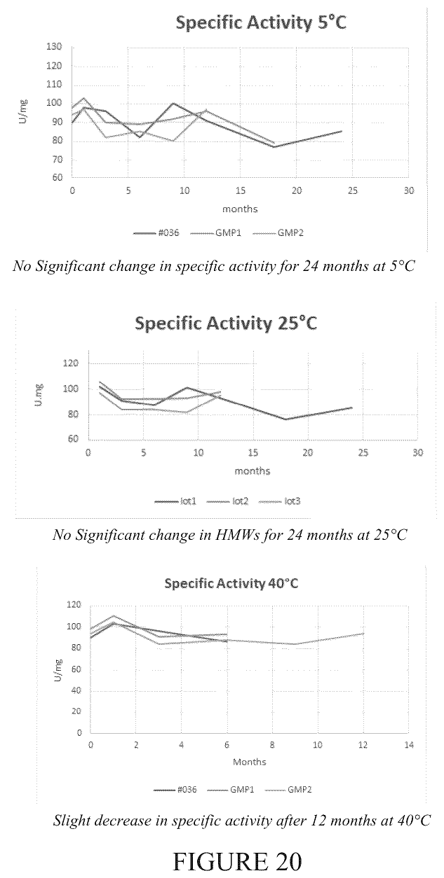

The specific activity for all preferred formulation samples corresponding to each time point and storage condition was assessed utilizing the enzymatic activity screen described above in Example 7. Briefly, the enzymatic assay used to evaluate the activity of each formulation sample was performed by mixing ENPP1-Fc with pNP-TMP. Product formation (pNP) is monitored spectrophotometrically at 405 nm for a period of 5 minutes, at 25° C. Specific activity of ENPP1-Fc is calculated by interpolating the change in absorbance over time obtained for each sample preparation to a standard curve of known concentrations of pNP. The activity data was plotted over time for each sample including a frozen control. Representative activity vs time plots for samples stored at 5° C., 25° C. and 40° C. up to 24 months are shown in FIG. 20. Similar results were seen for samples stored up to 36 months.

All samples across time points and storage conditions presented consistent levels of enzymatic activity. Thus, the experiments show that lyophilized ENPP1-Fc formulations remained stable under long term storage even when stored under different temperature conditions.

### Example 23: Stability Analysis of Reconstituted Samples in Vials and Syringes

The following assay was done to determine whether the reconstituted formulations when stored in vials and syringes for different time points undergo any loss of protein or loss of activity over a period of time.

Lyophilized ENPP1-Fc in preferred formulations were reconstituted in water to achieve different concentrations ranging from 1 mg/ml, 2.5 mg/ml and 10 mg/ml. The samples at different concentrations were incubated at 5° C. in two sets of syringes (high volume sample-250 μl and low volume sample-50 μl) for different time intervals ranging from 1-12 hours. The experiments were repeated in triplicates. A sample that was reconstituted and diluted to 10, 2.5, and 1 mg/mL at the time of assay was used as the control. The enzyme activity of an unincubated sample (time-0 hrs) was determined. The activity of the incubated samples (8-12 hrs) was then normalized against the control value. Protein recovery relative to the 50 mg/mL normalized starting concentrations was also measured for each sample following the protocols described above in prior examples.

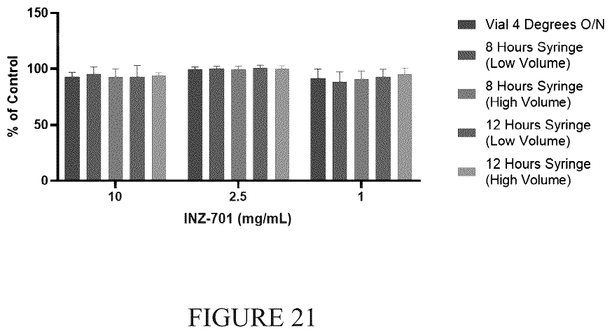

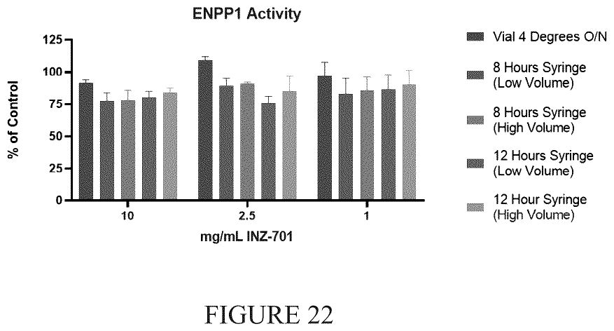

FIG. 21 shows the percent recovery for reconstituted ENPP1-Fc stored in syringes or vials at different time intervals. These data indicate that reconstituted ENPP1-Fc protein is stable when stored in syringes at different concentrations. The specific activity of reconstituted ENPP1-Fc samples was measured following the protocols described above in previous examples. FIG. 22 shows the summary enzymatic activity data for the reconstituted ENPP1-Fc polypeptide samples stored in syringes or vials at different time intervals. All samples across time points and storage conditions presented consistent levels of enzymatic activity. The protein activity was unchanged when stored in vials but there was a slight loss of activity when samples were stored in syringes for over 8 hours.

### Example 24: Stability Analysis Post Dilution of Reconstituted Samples

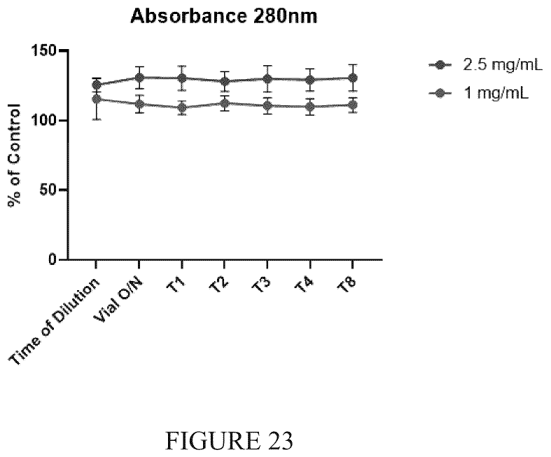

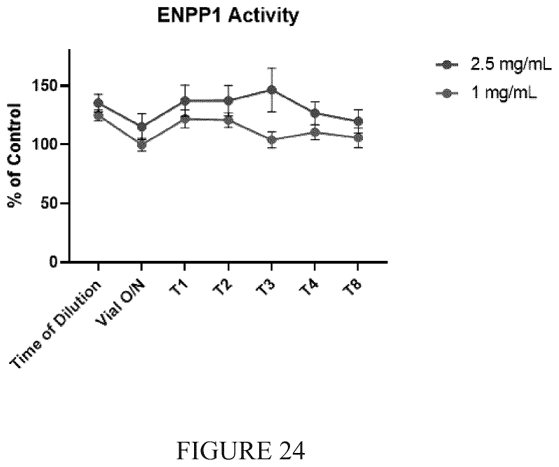

Lyophilized ENPP1-Fc formulations in vials are reconstituted to 25 mg/mL. Two sets of 2.5 mg/mL and 1 mg/mL dilutions are created. A control sample was created by taking a separate vial and diluting it to 2.5 mg/mL and 1 mg/mL at time of assay using a syringe. Hence the control sample will be at the incubation time of zero. The diluted samples 2.5 mg/ml and 1 mg/ml were incubated at 5° C. overnight and a subset of samples were incubated at room temperature (25° C.) for time intervals of 1 hr., 2 hr., 3 hr., 4 hr and 8 hr. Enzyme activity and protein concentrations were measured as described above in prior examples. The values were normalized against the control sample. FIG. 23 shows the percent recovery for reconstituted ENPP1-Fc stored in syringes or vials at different time intervals. The assay indicated that there is no loss in protein concentrations when stored overnight in vials at 5° C. or when stored in RT up to 8 hours. FIG. 24 shows the summary enzymatic activity data for the reconstituted ENPP1-Fc polypeptide samples stored in syringes or vials at different time intervals. The assay indicated that there is no loss in protein activity when stored overnight in vials at 5° C. or when stored in RT up to 4 hours. Minor loss of enzyme activity was observed when samples were stored in RT post 4 hours.

### REFERENCES

ATS. American Thoracic Society Statement: Guidelines for the six-minute walk test. AmJRespir, CritCare Med. 2002 2002; 166 (1): 111-7.
Brachet C, Mansbach A L, Clerckx A, Deltenre P, Heinrichs C. Hearing loss is part of the clinical picture of ENPP1 loss of function mutation. Horm Res Paediatr. 2014; 81 (1): 63-6.
CTFG. Recommendations related to contraception and pregnancy testing in clinical trials. 2014 [19 May 2020]; Available from: http://www.hma.eu/fileadmin/dateien/Human_Medicines/01-About_HMA/Working_Groups/CTFG/2014_09 HMA_CTFG_Contraception.pdf.
Ferreira C, Ziegler S, Gahl W. Generalized Arterial Calcification of Infancy. In: Adam M P, Ardinger H H, Pagon R A, Wallace S E, Bean L J H, Stephens K, et al., editors. GeneReviews®. Seattle (WA) 2014.
Guy W. The Clinical Global Impression Scale. In: Rush Jr A J, First M B, Blacker D, editors. Handbook of Psychiatric Measures, 2008. Washington, DC: American Psychiatric Publishing, Inc; 1976. p. 90-2.
IOF. Autosomal Recessive Hypophosphatemic Rickets Type 2 (ARHR2). 2019 [19 May 2020]; Available from: https://www.iofbonehealth.org/osteoporosis-musculoskeletal-disorders/skeletal-rare-disorders/autosomal-recessive-hypophosphatemi-0.
Mackenzie N C, Huesa C, Rutsch F, MacRae V E. New insights into NPP1 function: lessons from clinical and animal studies. Bone. 2012 November; 51 (5): 961-8.
McDonald C M, Henricson E K, Abresch R T, Florence J M, Eagle M, Gappmaier E, et al. The 6-minute walk test and other endpoints in Duchenne muscular dystrophy: longitudinal natural history observations over 48 weeks from a multicenter study. Muscle Nerve. 2013 September; 48 (3): 343-56.
Montes J, McDermott M P, Mirek E, Mazzone E S, Main M, Glanzman A M, et al. Ambulatory function in spinal muscular atrophy: Age-related patterns of progression. PLOS One. 2018; 13 (6): e0199657.
Moran J J. Idiopathic arterial calcification of infancy: a clinicopathologic study. Pathol Annu. 1975; 10:393-417.
Mylius C F, Paap D, Takken T. Reference value for the 6-minute walk test in children and adolescents: a systematic review. Expert Rev Respir Med. 2016 December; 10 (12): 1335-52.
NCI. National Cancer Institute Division of Cancer Treatment and Diagnosis (DCTD), National Cancer Institute (NCI), National Institutes of Health (NIH), Department of Health and Human Services (DHHS). Common Terminology Criteria for Adverse Events V5.0 (CTCAE). Published: Nov. 27, 2017. Available at https://ctep.cancer.gov/protocoldevelopment/electronic_applications/docs/ctcae_v5 quick_reference_8.5×11.pdf. 2017.
Orriss I R, Arnett T R, Russell R G. Pyrophosphate: a key inhibitor of mineralisation. Curr Opin Pharmacol. 2016 June; 28:57-68.
Rutsch F, Boyer P, Nitschke Y, Ruf N, Lorenz-Depierieux B, Wittkampf T, et al. Hypophosphatemia, hyperphosphaturia, and bisphosphonate treatment are associated with survival beyond infancy in generalized arterial calcification of infancy. Circ Cardiovasc Genet. 2008 December; 1 (2): 133-40.
Steichen-Gersdorf E, Lorenz-Depiereux B, Strom T M, Shaw N J. Early onset hearing loss in autosomal recessive hypophosphatemic rickets caused by loss of function mutation in ENPP1. J Pediatr Endocrinol Metab. 2015 July; 28 (7-8): 967-70.
van der Meijden J C, Kruijshaar M E, Harlaar L, Rizopoulos D, van der Beek N, van der Ploeg A T. Long-term follow-up of 17 patients with childhood Pompe disease treated with enzyme replacement therapy. J Inherit Metab Dis. 2018 November; 41 (6): 1205-14.

### INCORPORATION BY REFERENCE

All publications and patents mentioned herein are hereby incorporated by reference in their entirety as if each individual publication or patent was specifically and individually indicated to be incorporated by reference.

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1-277. (canceled)

278. A lyophilized polypeptide formulation, wherein the formulation comprises: (a) an ENPP1 polypeptide and (b) one or more of a buffering agent, a stabilizing agent, a salt, and a surfactant, and wherein said buffering agent is selected from the group consisting of succinate, citrate, bicarbonate, tris, or glycylglycine.

279. The formulation of claim 278, wherein the buffering agent maintains a pH range from pH 6-7 or pH7-8 when reconstituted in solution.

280. The formulation of claim 278, wherein the buffering agent comprises a concentration ranging from 5 mM to 100 mM when reconstituted in solution.

281. The formulation of claim 278, wherein the formulation comprises one or more pharmaceutically acceptable additives.

282. The formulation of claim 281, wherein the one or more pharmaceutically acceptable additive comprises stabilizing agents, amino acids, salts, metal ions, and surfactants.

283. The formulation of claim 282, wherein the pharmaceutically acceptable additive is a sugar and is selected from the group consisting of sucrose, trehalose, mannose, maltose, lactose, glucose, raffinose, cellobiose, gentiobiose, isomaltose, arabinose, glucosamine, fructose, mannitol, or sorbitol.

284. The formulation of claim 282, wherein the pharmaceutically acceptable additive is an amino acid and is selected from the group consisting of glycine, arginine, histidine, alanine, proline, serine, and glutamic acid.

285. The formulation of claim 282, wherein the pharmaceutically acceptable additive is a salt and is selected from the group consisting of sodium chloride (NaCl), Calcium chloride (CaCl₂), Zinc chloride (ZnCl₂), and/or Magnesium chloride (MgCl₂).

286. The formulation of claim 282, wherein the pharmaceutically acceptable additive is a surfactant and is selected from the group consisting of a polysorbate, poloxamer, triton, sodium dodecyl sulfate, sodium laurel sulfate, sodium octyl glycoside, lauryl sulfobetaine, myristyl-sulfobetaine, linoleyl-sulfobetaine, stearyl-sulfobetaine, laurylsarcosine, myristyl-sarcosine, linoleyl-sarcosine, stearyl-sarcosine, linoleyl-betaine, myristyl-betaine, cetyl-betaine, lauroam idopropyl-betaine, cocam idopropyl-betaine, linoleamidopropyl-betaine, myristam idopropyl-betaine, palm idopropyl-betaine, isostearam idopropyl-betaine, myristam idopropyl-dimethylamine, palm idopropyldimethylamine, isostearam idopropyl-dimethylamine, sodium methyl cocoyl-taurate, disodium methyl oleyl-taurate, dihydroxypropyl PEG 5 linoleammonium chloride, polyethylene glycol, polypropylene glycol, polysorbate 20, polysorbate 21, polysorbate 40, polysorbate 60, polysorbate 61, polysorbate 65, polysorbate 80, polysorbate 81, polysorbate 85, polysorbate 188, PEG3350, and mixtures thereof.

287. The formulation of claim 280, wherein the formulation comprises a buffering agent, a stabilizing agent, a salt, an amino acid, and a surfactant, and wherein the buffering agent is a citrate or a succinate, wherein the stabilizing agent is sucrose and/or mannitol, wherein the salt is calcium chloride (CaCl₂) or zinc chloride (ZnCl₂), and the surfactant is polysorbate 20 or polysorbate 80.

288. The formulation of claim 287, wherein the pH of the formulation is pH 6.2 to pH 6.5.

289. The formulation of claim 287, wherein the formulation comprises 15 to 25 mM of citrate, 75 mM to 95 mM each of sucrose, mannitol, or combinations thereof, 100 mM to 300 mM of sucrose, 1 mM to 3 mM of calcium chloride and 0.005% to 0.1% (w/v) of PS20.

290. The formulation of claim 278, wherein the formulation comprises an ENPP1 polypeptide cofactor and said ENPP1 polypeptide cofactor is selected from the group consisting of calcium chloride, calcium sulphate, zinc chloride, zinc sulphate and adenosine monophosphate.

291. The formulation of claim 290, wherein the ENPP1 polypeptide cofactor is present at a concentration ranging from 1 mM to 10 mM when reconstituted in solution.

292. The formulation of claim 287, wherein the surfactant is present at a concentration ranging from 0.02% to 0.10% (w/v) when reconstituted in solution.

293. The formulation of claim 287, wherein the ENPP1 polypeptide comprises an amino acid sequence that is at least 75%, 80%, 85%, 90%, 91%, 92%, 93%, 94%, 95%, 96%, 97%, 98%, 99%, or 100% identical to an amino acid sequence comprising SEQ ID NO: 2, SEQ ID NO: 13, SEQ ID NO: 14, or SEQ ID NO: 15.

294. The formulation of claim 287, wherein the ENPP1 polypeptide is a fusion protein comprising a soluble ENPP1 polypeptide domain and heterologous protein portion comprises an Fc domain, and wherein said heterologous protein portion increases the circulating half-life of the soluble ENPP1 polypeptide in a mammal.

295. The formulation of claim 294, wherein the Fc domain comprises an amino acid sequence that is at least 75%, 80%, 85%, 90%, 91%, 92%, 93%, 94%, 95%, 96%, 97%, 98%, 99%, or more identical to the amino acid sequence of SEQ ID NO: 12.

296. The formulation of claim 287, wherein the ENPP1 polypeptide further comprises a heterologous moiety and said heterologous moiety is selected from the group consisting of a glycosylated amino acid, a PEGylated amino acid, a farnesylated amino acid, an acetylated amino acid, a biotinylated amino acid, and a lipid moiety.

297. The formulation of claim 278, wherein the buffering agent:
(i) increases the onset temperature of aggregate formation;
(ii) increases the onset temperature of aggregate formation by at least 2° C.;
(iii) decreases formation of high molecular weight species; and/or
(iv) decreases formation of high molecular weight species by at least 10%, 15%, 20%, 25%, 30%, 35%, 40%, 45%, 50%.

298. The formulation of claim 282, wherein the pharmaceutical additive:
(i) increases resistance to physical stress;
(ii) decreases formation of high molecular weight species by at least 10%, 15%, 20%, 25%, 30%, 35%, 40%, 45%, 50%.

299. The formulation of claim 287, wherein the formulation comprises trisodium citrate dihydrate, calcium chloride, sucrose, mannitol and polysorbate 20.

300. The formulation of claim 299, wherein the formulation comprises per 0.5 ml final reconstituted volume: about 25 mg of ENPP1 polypeptide, about 2.94 mg trisodium citrate dihydrate, about 0.15 mg calcium chloride dihydrate, about 30 mg of sucrose, about 7.5 mg D (−) mannitol, and about 0.25 mg polysorbate.

301. The formulation of claim 300, wherein the formulation is reconstituted in a sterile injectable solution, wherein:
(i) the formulation is reconstituted in a reconstitution solution or sterile water, preferably wherein the reconstitution solution comprises a pharmaceutically acceptable carrier and more preferably an additive, wherein the pharmaceutically acceptable carrier is selected from saline solution, purified water, or sterile water for injection; or
(ii) the formulation is completely reconstituted within a period of less than 100 seconds, 80 seconds, 70 seconds, 68 seconds, 65 seconds, or 60 seconds; and
(iii) the reconstituted formulation comprises: at least or about 50 mg/mL of the ENPP1 polypeptide, about 20 mM citrate at about pH 6.3, about 2 mM calcium chloride, about 175 mM sucrose, about 82 mM mannitol, and about 0.05% w/v polysorbate,
or
wherein the reconstituted formulation comprises: at least or about 50 mg/mL of the ENPP1 polypeptide, about 20 mM citrate at about pH 6.3, about 2 mM calcium chloride, about 175 mM sucrose, about 82 mM (D) mannitol, and about 0.05% w/v polysorbate 20
or
wherein the reconstituted formulation comprises: at least or about 50 mg/ml of the ENPP1 polypeptide, about 20 mM citrate at about pH 6.3, about 88 mM sucrose, about 82 mM mannitol, about 2 mM calcium chloride, and about 0.05% polysorbate 20
or
wherein the reconstituted formulation comprises: at least or about 50 mg/mL of the ENPP1 polypeptide, about 20 mM citrate at about pH 6.3, about 263 mM sucrose, about 2 mM calcium chloride, and about 0.05% polysorbate 20.

302. The formulation of claim 300, wherein the formulation exhibits long term stability at −80° C. to 40° C. or wherein the formulation has a shelf life of at least 3, 6, 12, 24, 36, 48, or 60 months.

303. The formulation of claim 301, wherein the reconstituted formulation has a shelf life of at least 1, 2, 3, 4, 5, 6, 12, 18, 24, 48, or 60 hours.

304. The formulation of claim 301, wherein the reconstituted formulation is administered:
(i) parenterally; via subcutaneous injection; via intravenous injection; via intradermal injection; or via intramuscular injection;
(ii) wherein the reconstituted formulation is self-administered;
and/or
(iii) wherein the reconstituted formulation is administered several times daily, every two days, three days, one week, or one month, optionally wherein a second dosage of the formulation is administered after a suitable time interval of at least after two days, after four days, after a week, or after a month.

305. The formulation of claim 278, wherein the formulation comprises one of formulations A-E.

306. A method for generating a pharmaceutical solution comprising an ENPP1 polypeptide, the method comprising contacting the lyophilized polypeptide formulation of claim 23 with a sterile reconstitution solution or sterile water to thereby generate a reconstituted solution comprising the ENPP1 polypeptide, wherein;
(i) the reconstitution solution comprises a pharmaceutically acceptable carrier and/or an additive, optionally wherein the pharmaceutically acceptable carrier is selected from saline solution, purified water, or sterile water for injection;
(ii) the formulation is completely reconstituted within a period of less than 100 seconds, 80 seconds, 70 seconds, 68 seconds, 65 seconds, or 60 seconds;
and/or
(iii) the reconstituted formulation comprises: at least or about 50 mg/mL of the ENPP1 polypeptide, about 20 mM citrate at about pH 6.3, about 2 mM calcium chloride, about 175 mM sucrose, about 82 mM mannitol, and about 0.05% w/v polysorbate,
or
the reconstituted formulation comprises: at least or about 50 mg/mL of the ENPP1 polypeptide, about 20 mM citrate at about pH 6.3, about 2 mM calcium chloride, about 175 mM sucrose, about 82 mM (D) mannitol, and about 0.05% w/v polysorbate 20,
or
the reconstituted formulation comprises: at least or about 50 mg/mL of the ENPP1 polypeptide, about 20 mM citrate at about pH 6.3, about 88 mM sucrose, about 82 mM mannitol, about 2 mM calcium chloride, and about 0.05% polysorbate 20,
or
the reconstituted formulation comprises: at least or about 50 mg/mL of the ENPP1 polypeptide, about 20 mM citrate at about pH 6.3, about 263 mM sucrose, about 2 mM calcium chloride, and about 0.05% polysorbate 20.

307. A vial or a syringe comprising the lyophilized formulation of claim 300.

308. A method for:
(i) preventing the progression of or reducing vascular calcification in a subject in need thereof, the method comprising: administering to the subject an effective amount of the reconstituted formulation according to claim 23, to thereby prevent the progression of or reduce vascular calcification in the subject, or
(ii) preventing the progression of or reducing pathological calcification in a subject with ENPP1 Deficiency, the method comprising: administering to the subject an effective amount of the reconstituted formulation according to claim 23, to thereby prevent the progression of or reduce pathological calcification in the subject, or
(iii) preventing the progression of or reducing tissue calcification in a subject in need thereof, the method comprising: administering to the subject an effective amount of the reconstituted formulation according to claim 23, to thereby prevent the progression of or reduce tissue calcification in the subject, or
(iv) for increasing circulating pyrophosphate (PPi) in a subject in need thereof, the method comprising: administering to the subject an effective amount of the reconstituted formulation according to claim 23, to thereby increase circulating PPi in the subject, or
(v) for increasing pyrophosphatase activity in a subject in need thereof, the method comprising: administering to the subject an effective amount of the reconstituted formulation according to claim 23, to thereby increase circulating PPi in the subject.

309. The method of claim 308, wherein the subject has ENPP1 Deficiency, a disorder involving pathological calcification, a disorder involving pathological ossification or ABCC6 Deficiency.

310. The method of claim 308, wherein the subject has or is at risk for developing pathological soft tissue calcification, arterial calcification, vascular calcification, chronic kidney disease (CKD), end stage renal disease (ESRD), Pseudoxanthoma Elasticum (PXE), calcific uremic arteriolopathy (CUA), calciphylaxis, ossification of the posterior longitudinal ligament (OPLL), or hypophosphatemic rickets

311. The method of claim 308, wherein said formulation is reconstituted in sterile water and then administered parenterally through one of subcutaneous injection, intravenous injection, intradermal injection and intramuscular injection.

312. The method of claim 311, wherein the formulation is administered several times daily, every two days, three days, one week, or one month, optionally wherein a second dosage of the formulation is administered after a suitable time interval of at least after two days, after four days, after a week, or after a month.


SEQUENCE LISTING (115 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 827 aa, synthetic construct
  REGION 1..827: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 3 - protein, 16 aa, synthetic construct
  REGION 1..16: note = ENPP2 Signal sequence
LFTFAVGVNICLGFTA

SEQ ID NO: 4 - protein, 906 aa, Mus musculus
MERDGDQAGHGPRHGSAGNGRELESPAAASLLAPMDLGEEPLEKAERARPAKDPNTYKVLSLVLSVCVLTTILGCIFGLKPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 5 - protein, 924 aa, Bos taurus
MERDSCAGGGSRGGEGGRGPREGLAGNGRDPGPGRAAEASGEPQAAASLLAPMDLGEEPLERAARARPAKDPNTYKVLSLVLSVCVLTTILGCIFGLKPSCAKEIKSCKGRCFERTFGNCRCDAACVDLGNCCLDYQETCIEPERIWTCTKFRCGEKRLSRSLCSCSDDCKDKGDCCINHGSVCRGEKSWAEEECDSIDEPQCPAGFETPPTLLFSLDGFRAEYLHTWGGLLPVISKLKTCGTYTKNMRPVYPTKTFPNHYSIVTGLYPESHGIIDNNIYDPQMNANFALKNKEKFNPEWYKGEPIWLTAKYQGLKTGTFFWPGSDVKINGIFPDIYKIYNVSVPFEERILAILKWLQLPKDERPHFYTLYLEEPDSSGHSYGPVSSEVIRALQRVDNMVGMLMDGLKELNLHRCLNLILISDHGMEQGSCKKYVYLNKYLGDTKDYKVVYGPAARLRPSDVPDKYYSFDYEGIAKNLSCQEPNQHFKPYLKHFLPKRLHFAKNDRIERLTFYLDPQWQLALNPSERKYCGGGFHGSDNTFLNMQALFIGYGPGFKHSTEVDSFENIEVYNLMCDLLNLTPAPNNGTHGSLNHLLSNPVYTPKHPKEVRPLVQCPFTRAPRESLDCSCDPSILPIVDFQTQLNLTMAEEKTIKRGALPYGRPRVLQNSTVCLLYQHQFVSGYSRDILMPLWTSYTIGRNDSFSTEDFSNCLYQDLRIPLSPVHKCSFYKNNAKLSYGLLSPPQLHKGSSQVYSEALLTTNIVPMYQSFQVIWHYLHGTLLQRYAEERNGLNVVSGPVFDSDYDGRYDSLETLKQNSKIIRNLEVLIPTHFFLVLTSCKNTSQTPLQCENLDAMAFILPHKTDNSESCAHGKHESLWVEELLKLHTARITDVEHITGLSFYQERKEPISDILKLKTHLPTFNQED

SEQ ID NO: 6 - protein, 925 aa, Oryctolagus cuniculus
MERDGCAGGGSRGGEGGRAPREGPAGNSRDPGRSHAAEAPGNPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDQGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDNMVGMLMDGLKELNLHRCLNLILVSDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNIFSNMQALFVGYGPGFKHGIEVDTFENIEVYNLMCDLLNLTPAPNNGTHGSLNHLLKNPVYTPKHPKEVHPLIQCPFTRNPRDNLGCSCNPSILPIEDFQTQFNLTVAEEKNIKHETLPYGRPRVLQKKNTICLLSQHQFMSGYSQDILMPLWTSYTVDRNDSFSTEDFSNCLYQDFRISLSPVHKCSFYKNNTKVSYGFLSPPQLNKNSRGIYSEALLTTNIVPMYQSFQVIWRYFHDTLLRKYAEERNGVNVVSGPVFDFDYDGRYDSLEILRQKRRVIRNQEILIPTHFFIVLTSCKDASQTPLHCENLDTLAFILPHRTDNSESCLHGKHESSWVEELLMLHRARITDVEHITGLSFYQQRKEPVSDILKLKTHLPTFSQED

SEQ ID NO: 7 - protein, 19 aa, synthetic construct
  REGION 1..19: note = Azurocidin Signal sequence
MTRLTVLALLAGLLASSRA

SEQ ID NO: 8 - protein, 926 aa, Papio hamadryas
MERDGCAGGGSQGGGKGGRGPREGLAGNGRDPSHGQASEAPGDPQAAASLLAPMDLGEEPLEKAAGARPAKDPNTYKVLSLVLSVCVLTTILGCIFGLKPSCAKEVKSCKGRCFERTFGNCRCDVACVDLGNCCLDYQETCIEPERIWTCNKFRCGEKRLSRSLCACSDDCKERGDCCINYSAVCQGEKSWVEETCENINEPQCPEGFEMPPTLLFSLDGFRAEYLHTWGGLLPVISKLKKCGTYAKNMRPVYPTKTFPNHYSIVTGLYPESHGIIDNKMYDPKMNASFSLKSKEKFNPEWYKGEPIWLTAKYQGLRSGTFFWPGSDVKINGIFPDIYKIYNGSVPFEERILAILKWLRLPKDERPHFYTLYLEEPDSSGHSYGPVSSEVIKALQRVDNMVGMLMDGLKELNLHQCLNLILISDHGMEQGSCKKYIYLNKYLGDTKNIKVIYGPAARLRPSDVPEKYYSFNYENIARNLSCREPNQHFKPYLKHFLPKRLHFAKSDRIEPLTFYLDPQWQLALSPSERKYCGSGFHGSDNVFSNMQALFVGYGPGFQHGIEVDSFENIEVYNLMCDLLNLTPAPNNGTHGSLNHLLKNPIYTPKHPKEVQPSVQCPLAGSPRDSLGCSCNPSILPIVDFQTQFNLTTAEEKNINRASLPYGRPRLLQKKSSVCLLYQHQFVSGYSHDVLMPLWTSYTVNRNDSFSTEDFSNCLYQDLRISFSPIHNCSFYKNNAKLSYGFLSPPQLSKDSSQIYSEALLTSNIVPMYQSFQVIWRYFHDTLLQRYAEERNSINVVSGPVFDSDYDGRYDSSEALKRNRRVIRNQEILIPTHFFIVITSCKNTSQTPLQCDNLDPLAFILPHRSDNSESCVHEKRESSWIEELLMMHRARIMDVEHITGLSFYQERKEPVSDILKLKTHLPTVSQED

SEQ ID NO: 9 - protein, 1063 aa, synthetic construct
  REGION 1..1063: note = ENPPI Polypeptide
FTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 1060 aa, synthetic construct
  REGION 1..1060: note = ENPP1 Polypeptide
GLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 1057 aa, synthetic construct
  REGION 1..1057: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 227 aa, synthetic construct
  REGION 1..227: note = Human IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 832 aa, synthetic construct
  REGION 1..832: note = ENPP1 Polypeptide
TAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 14 - protein, 830 aa, synthetic construct
  REGION 1..830: note = ENPP1 Polypeptide
GLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 15 - protein, 827 aa, synthetic construct; identical to SEQ ID NO: 2
  REGION 1..827: note = ENPP1 Polypeptide

SEQ ID NO: 16 - protein, 20 aa, synthetic construct
  REGION 1..20: note = ENPP7 Signal sequence
MRGPAVLLTVALATLLAPGA

SEQ ID NO: 17 - protein, 22 aa, synthetic construct
  REGION 1..22: note = ENPP7 Signal sequence
MRGPAVLLTVALATLLAPGAGA

SEQ ID NO: 18 - protein, 22 aa, synthetic construct
  REGION 1..22: note = ENPP5 Signal sequence
MTSKFLLVSFILAALSLSTTFS

SEQ ID NO: 19 - protein, 98 aa, synthetic construct
  REGION 1..98: note = ENPP121GLK Signal sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLK

SEQ ID NO: 20 - protein, 94 aa, synthetic construct
  REGION 1..94: note = ENPP121 protein export signal sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFT

SEQ ID NO: 21 - protein, 4 aa, synthetic construct
  REGION 1..4: note = Linker
GGGA

SEQ ID NO: 22 - protein, 4 aa, synthetic construct
  REGION 1..4: note = Linker
GGGS

SEQ ID NO: 23 - protein, 4 aa, synthetic construct
  REGION 1..4: note = Linker
GGGG

SEQ ID NO: 24 - protein, 5 aa, synthetic construct
  REGION 1..5: note = Linker
GGGGA

SEQ ID NO: 25 - protein, 5 aa, synthetic construct
  REGION 1..5: note = Linker
GGGGS

SEQ ID NO: 26 - protein, 5 aa, synthetic construct
  REGION 1..5: note = Linker
GGGGG

SEQ ID NO: 27 - protein, 4 aa, synthetic construct
  REGION 1..4: note = Linker
GGAG

SEQ ID NO: 28 - protein, 4 aa, synthetic construct
  REGION 1..4: note = Linker
GGSG

SEQ ID NO: 29 - protein, 4 aa, synthetic construct
  REGION 1..4: note = Linker
AGGG

SEQ ID NO: 30 - protein, 5 aa, synthetic construct
  REGION 1..5: note = Linker
SGGGG

SEQ ID NO: 31 - protein, 4 aa, synthetic construct
  REGION 1..4: note = Linker
SGGG

SEQ ID NO: 32 - protein, 4 aa, synthetic construct
  REGION 1..4: note = Linker
GAGA

SEQ ID NO: 33 - protein, 4 aa, synthetic construct
  REGION 1..4: note = Linker
GSGS

SEQ ID NO: 34 - protein, 6 aa, synthetic construct
  REGION 1..6: note = Linker
GAGAGA

SEQ ID NO: 35 - protein, 6 aa, synthetic construct
  REGION 1..6: note = Linker
GSGSGS

SEQ ID NO: 36 - protein, 8 aa, synthetic construct
  REGION 1..8: note = Linker
GAGAGAGA

SEQ ID NO: 37 - protein, 8 aa, synthetic construct
  REGION 1..8: note = Linker
GSGSGSGS

SEQ ID NO: 38 - protein, 10 aa, synthetic construct
  REGION 1..10: note = Linker
GAGAGAGAGA

SEQ ID NO: 39 - protein, 10 aa, synthetic construct
  REGION 1..10: note = Linker
GSGSGSGSGS

SEQ ID NO: 40 - protein, 12 aa, synthetic construct
  REGION 1..12: note = Linker
GAGAGAGAGAGA

SEQ ID NO: 41 - protein, 12 aa, synthetic construct
  REGION 1..12: note = Linker
GSGSGSGSGSGS

SEQ ID NO: 42 - protein, 6 aa, synthetic construct
  REGION 1..6: note = Linker
GGAGGA

SEQ ID NO: 43 - protein, 6 aa, synthetic construct
  REGION 1..6: note = Linker
GGSGGS

SEQ ID NO: 44 - protein, 9 aa, synthetic construct
  REGION 1..9: note = Linker
GGAGGAGGA

SEQ ID NO: 45 - protein, 9 aa, synthetic construct
  REGION 1..9: note = Linker
GGSGGSGGS

SEQ ID NO: 46 - protein, 12 aa, synthetic construct
  REGION 1..12: note = Linker
GGAGGAGGAGGA

SEQ ID NO: 47 - protein, 12 aa, synthetic construct
  REGION 1..12: note = Linker
GGSGGSGGSGGS

SEQ ID NO: 48 - protein, 4 aa, synthetic construct
  REGION 1..4: note = Linker
GGAG

SEQ ID NO: 49 - protein, 4 aa, synthetic construct
  REGION 1..4: note = Linker
GGSG

SEQ ID NO: 50 - protein, 4 aa, synthetic construct
  REGION 1..4: note = Linker
GGAG

SEQ ID NO: 51 - protein, 4 aa, synthetic construct
  REGION 1..4: note = Linker
GGSG

SEQ ID NO: 52 - protein, 8 aa, synthetic construct
  REGION 1..8: note = Linker
GGAGGGAG

SEQ ID NO: 53 - protein, 8 aa, synthetic construct
  REGION 1..8: note = Linker
GGSGGGSG

SEQ ID NO: 54 - protein, 12 aa, synthetic construct
  REGION 1..12: note = Linker
GGAGGGAGGGAG

SEQ ID NO: 55 - protein, 12 aa, synthetic construct
  REGION 1..12: note = Linker
GGSGGGSGGGSG

SEQ ID NO: 56 - protein, 5 aa, synthetic construct
  REGION 1..5: note = Linker
GGGGA

SEQ ID NO: 57 - protein, 5 aa, synthetic construct
  REGION 1..5: note = Linker
GGGGS

SEQ ID NO: 58 - protein, 15 aa, synthetic construct
  REGION 1..15: note = Linker
GGGGAGGGGAGGGGA

SEQ ID NO: 59 - protein, 15 aa, synthetic construct
  REGION 1..15: note = Linker
GGGGSGGGGSGGGGS

SEQ ID NO: 60 - protein, 5 aa, synthetic construct
  REGION 1..5: note = Linker
GGGAG

SEQ ID NO: 61 - protein, 6 aa, synthetic construct
  REGION 1..6: note = Linker
GGGAGG

SEQ ID NO: 62 - protein, 7 aa, synthetic construct
  REGION 1..7: note = Linker
GGGAGGG

SEQ ID NO: 63 - protein, 5 aa, synthetic construct
  REGION 1..5: note = Linker
TGGGG

SEQ ID NO: 64 - protein, 4 aa, synthetic construct
  REGION 1..4: note = Linker
AAAL

SEQ ID NO: 65 - protein, 4 aa, synthetic construct
  REGION 1..4: note = Linker
AAAK

SEQ ID NO: 66 - protein, 4 aa, synthetic construct
  REGION 1..4: note = Linker
AAAR

SEQ ID NO: 67 - protein, 14 aa, synthetic construct
  REGION 1..14: note = Linker
EGKSSGSGSESKST

SEQ ID NO: 68 - protein, 12 aa, synthetic construct
  REGION 1..12: note = Linker
GSAGSAAGSGEF

SEQ ID NO: 69 - protein, 12 aa, synthetic construct
  REGION 1..12: note = Linker
AEAAAKEAAAKA

SEQ ID NO: 70 - protein, 18 aa, synthetic construct
  REGION 1..18: note = Linker
KESGSVSSEQLAQFRSLD

SEQ ID NO: 71 - protein, 10 aa, synthetic construct
  REGION 1..10: note = Linker
GENLYFQSGG

SEQ ID NO: 72 - protein, 8 aa, synthetic construct
  REGION 1..8: note = Linker
SACYCELS

SEQ ID NO: 73 - protein, 5 aa, synthetic construct
  REGION 1..5: note = Linker
RSIAT

SEQ ID NO: 74 - protein, 17 aa, synthetic construct
  REGION 1..17: note = Linker
RPACKIPNDLKQKVMNH

SEQ ID NO: 75 - protein, 36 aa, synthetic construct
  REGION 1..36: note = Linker
GGSAGGSGSGSSGGSSGASGTGTAGGTGSGSGTGSG

SEQ ID NO: 76 - protein, 17 aa, synthetic construct
  REGION 1..17: note = Linker
AAANSSIDLISVPVDSR

SEQ ID NO: 77 - protein, 36 aa, synthetic construct
  REGION 1..36: note = Linker
GGSGGGSEGGGSEGGGSEGGGSEGGGSEGGGSGGGS

SEQ ID NO: 78 - protein, 15 aa, synthetic construct
  REGION 1..15: note = Linker
  REGION 1..15: note = MISC_FEATURE - Features section of the listing that any one or all of aminoacids 1-15 can either be present or absent
RRRRRRRRRRRRRRR

SEQ ID NO: 79 - protein, 16 aa, synthetic construct
  REGION 1..16: note = Linker
DSSSEEKFLRRIGRFG

SEQ ID NO: 80 - protein, 12 aa, synthetic construct
  REGION 1..12: note = Linker
EEEEEEEPRGDT

SEQ ID NO: 81 - protein, 12 aa, synthetic construct
  REGION 1..12: note = Linker
APWHLSSQYSRT

SEQ ID NO: 82 - protein, 12 aa, synthetic construct
  REGION 1..12: note = Linker
STLPIPHEFSRE

SEQ ID NO: 83 - protein, 12 aa, synthetic construct
  REGION 1..12: note = Linker
VTKHLNQISQSY

SEQ ID NO: 84 - protein, 15 aa, synthetic construct
  REGION 1..15: note = Linker
  REGION 1..15: note = MISC_FEATURE - Features section of the listing that any one or all of aminoacids 2-15 can either be present or absent
EEEEEEEEEEEEEEE

SEQ ID NO: 85 - protein, 7 aa, synthetic construct
  REGION 1..7: note = Linker
RSGSGGS

SEQ ID NO: 86 - protein, 15 aa, synthetic construct
  REGION 1..15: note = Linker
  REGION 1..15: note = MISC_FEATURE - Features section of the listing that any one or all of aminoacids 2-15 can either be present or absent
DDDDDDDDDDDDDDD

SEQ ID NO: 87 - protein, 16 aa, synthetic construct
  REGION 1..16: note = Linker
LVIMSLGLGLGLGLRK

SEQ ID NO: 88 - protein, 15 aa, synthetic construct
  REGION 1..15: note = Linker
VIMSLGLGLGLGLRK

SEQ ID NO: 89 - protein, 14 aa, synthetic construct
  REGION 1..14: note = Linker
IMSLGLGLGLGLRK

SEQ ID NO: 90 - protein, 13 aa, synthetic construct
  REGION 1..13: note = Linker
MSLGLGLGLGLRK

SEQ ID NO: 91 - protein, 12 aa, synthetic construct
  REGION 1..12: note = Linker
SLGLGLGLGLRK

SEQ ID NO: 92 - protein, 11 aa, synthetic construct
  REGION 1..11: note = Linker
LGLGLGLGLRK

SEQ ID NO: 93 - protein, 10 aa, synthetic construct
  REGION 1..10: note = Linker
GLGLGLGLRK

SEQ ID NO: 94 - protein, 9 aa, synthetic construct
  REGION 1..9: note = Linker
LGLGLGLRK

SEQ ID NO: 95 - protein, 8 aa, synthetic construct
  REGION 1..8: note = Linker
GLGLGLRK

SEQ ID NO: 96 - protein, 7 aa, synthetic construct
  REGION 1..7: note = Linker
LGLGLRK

SEQ ID NO: 97 - protein, 6 aa, synthetic construct
  REGION 1..6: note = Linker
GLGLRK

SEQ ID NO: 98 - protein, 5 aa, synthetic construct
  REGION 1..5: note = Linker
LGLRK

SEQ ID NO: 99 - protein, 4 aa, synthetic construct
  REGION 1..4: note = Linker
GLRK

SEQ ID NO: 100 - protein, 15 aa, synthetic construct
  REGION 1..15: note = Linker
  REGION 1..15: note = MISC_FEATURE - Features section of the listing that any one or all of aminoacids 2-15 can either be present or absent
KKKKKKKKKKKKKKK

SEQ ID NO: 101 - protein, 5 aa, synthetic construct
  REGION 1..5: note = Linker
EAAAK

SEQ ID NO: 102 - protein, 5 aa, synthetic construct
  REGION 1..5: note = Linker
PAPAP

SEQ ID NO: 103-115 - skipped (13 entries)